(19) United States
(12) Patent Application Publication
(10) Pub. No.: US 2026/0069541 A1
(43) Pub. Date: Mar. 12, 2026

(54) COMPOSITIONS AND METHODS FOR THE DELIVERY OF ACTIVE AGENTS INCLUDING NUCLEIC ACIDS

(71) Applicants: Ohio State Innovation Foundation, Columbus, OH (US); THE WHITEOAK GROUP, INC., Rockville, MD (US)

(72) Inventors: Robert J. LEE, Lewis Center, OH (US); Yingwen HU, Gaithersburg, MD (US)

(21) Appl. No.: 19/106,465

(22) PCT Filed: Aug. 25, 2023
(86) PCT No.: PCT/US2023/031171

§ 371 Date: Feb. 25, 2025

Related U.S. Application Data
(60) Provisional application No. 63/401,113, filed on Aug. 25, 2022.

(51) Int. Cl. A61K 9/1272; A61K 9/00; A61K 9/51; A61K 31/7105; A61K 45/06; A61P 35/00; B82Y 5/00; C12N 15/113

(52) U.S. Cl. CPC A61K 9/1272; A61K 9/0019; A61K 9/5123; A61K 31/7105; A61K 45/06; A61P 35/00; C12N 15/1137; B82Y 5/00; C12N 2310/14; C12Y 207/11001

(57) ABSTRACT

Disclosed herein are lipid nanoparticles for the delivery of active agents, including nucleic acids, as well as methods of making using thereof.

### CROSS-REFERENCE TO RELATED APPLICATIONS

This application claims benefit of U.S. Provisional Application No. 63/401,113, filed Aug. 25, 2022, which is hereby incorporated herein by reference in its entirety.

### BACKGROUND

Therapeutic nucleic acids include, e.g., small interfering RNA (siRNA), micro RNA (miRNA), antisense oligonucleotides, ribozymes, plasmids, and immune stimulating nucleic acids. These nucleic acids act via a variety of mechanisms. In the case of siRNA or miRNA, these nucleic acids can down-regulate intracellular levels of specific proteins through a process termed RNA interference (RNAi). Following introduction of siRNA or miRNA into the cell cytoplasm, these double-stranded RNA constructs can bind to a protein termed RISC. The sense strand of the siRNA or miRNA is displaced from the RISC complex providing a template within RISC that can recognize and bind mRNA with a complementary sequence to that of the bound siRNA or miRNA. Having bound the complementary mRNA the RISC complex cleaves the mRNA and releases the cleaved strands. RNAi can provide down-regulation of specific proteins by targeting specific destruction of the corresponding mRNA that encodes for protein synthesis.

The therapeutic applications of RNAi are extremely broad, since siRNA and miRNA constructs can be synthesized with any nucleotide sequence directed against a target protein. To date, siRNA constructs have shown the ability to specifically down-regulate target proteins in both in vitro and in vivo models. In addition, siRNA constructs are currently being evaluated in clinical studies.

In spite of recent progress, there remains a need in the art for improved lipid-therapeutic nucleic acid compositions that are suitable for general therapeutic use. These compositions would, for example, encapsulate nucleic acids with high-efficiency, have high drug:lipid ratios, protect the encapsulated nucleic acid from degradation and clearance in serum, be suitable for systemic delivery, and provide intracellular delivery of the encapsulated nucleic acid. In addition, these lipid-nucleic acid particles should be well-tolerated and provide an adequate therapeutic index, such that patient treatment at an effective dose of the nucleic acid is not associated with significant toxicity and/or risk to the patient.

### SUMMARY

Lipid nanoparticles (LNPs) have been shown to be effective delivery vehicles of mRNA, as shown by the successes of mRNA-based COVID-19 vaccines developed by BioNTech and Moderna. However, the intramuscular delivery of the LNP using current formulations results in systemic gene expression, which is related to rare but significant adverse effects such as myocarditis.

LNP compositions, referred to as QTsomes, combine a quaternary-amine lipid with a tertiary-amine lipid in the blend of lipids forming the LNP. When investigated previously, QTsomes showed excellent activity in anti-miR delivery and antisense oligo delivery. However, these LNP compositions were not optimized for intramuscular mRNA delivery.

Herein, key formulation parameters were examined and improved QTsome compositions were identified. These LNP compositions can exhibit superior intramuscular mRNA delivery efficiency. In some embodiments, these LNP compositions can exhibit superior intramuscular mRNA delivery efficiency while also decreasing concomitant systemic gene expression.

Through investigation, certain parameters for QTsome design were uncovered. For example, the presence of a cationic lipid (e.g., a quaternary amine-containing lipid such as DOTAP) in the LNP generally reduces systemic gene expression. Increasing percentages of cationic lipid (e.g., DOTAP) in the LNP generally reduces systemic gene expression significantly. At relatively high mole % cationic lipid (e.g., 5-8% DOTAP), muscular gene expression is reduced along with systemic expression. At relatively low mole % cationic lipid (e.g., 1.5-3% DOTAP), muscular gene expression is not significantly diminished; however, systemic gene expression is diminished. This suggests that a relatively low mole % cationic lipid (e.g., from 0.5 mol % to 3.5 mol %, or from 1.5 mol % to 3.0 mol % cationic lipid) is unexpectedly beneficial for achieving high intramuscular mRNA vaccine delivery without inducing side effects associated with systemic delivery, such as myocarditis. This is a key advantage with high potential impact for developing next generation of mRNA vaccines.

In the case of helper lipids, it was also discovered that DOPE was better than DSPC or DOPC for activity. Accordingly, in some embodiments, the LNPs described herein can comprise DOPE as a helper lipid.

It was also discovered that the preparation of LNPs at elevated temperature resulted in a dramatic loss of gene expression. Accordingly, in some embodiments, the LNP compositions described herein can be prepared at temperatures below 65° C. (e.g., below 60° C., below 55° C., below 50° C., below 45° C., below 40° C., below 35° C., or below 30° C., below 25° C., or below 20° C.).

It was also discovered that a 2-step mixing process in which the ethanol content of the lipid side is approximately 25% at the point of mixing resulted in superior LNP delivery efficiency relative to a single step 1:3 volume mixing, which has been standard practice in the LNP field. Accordingly, in some embodiments, the LNP compositions described herein can be prepared using a 2-step mixing process in which the ethanol content of the lipid side is approximately 25% at the point of mixing.

It was also discovered that ionizable lipid A066 improved mRNA delivery Accordingly, in some embodiments, the LNPs described herein can comprise A066. In some embodiments, A066 can be used in place of other ionizable lipids, such as SM-102.

It was also discovered that the addition of a cationic lipid (e.g., a quaternary amine-containing lipid such as DOTAP) changes the apparent pKa of the ionizable lipid, as determined by a TNS pKa assay. As a result, the addition of a cationic lipid (e.g., a quaternary amine-containing lipid such as DOTAP) can be used to fine-tune the apparent pKa value of LNPs to optimize for local or systemic mRNA, and/or to target delivery to certain tissues in the body.

It was also discovered that compositions comprising 1.5-3 mol % of a cationic lipid (e.g., a quaternary amine-containing lipid such as DOTAP), 1.5-2 mol % of a PEGylated lipid (e.g., mPEG-DMG), an ionizable lipid (e.g., A066, DODMA, MC-3, and/or SM-102), and a helper lipid (e.g., DOPE, DSPC, and/or DOPC) were particularly suitable for nucleic acid delivery including mRNA vaccine delivery for COVID-19, other viruses, or cancer.

Accordingly, provided herein are pharmaceutical compositions that comprise a lipid particle encapsulating an active agent. The lipid particle can comprise one or more cationic lipids; one or more ionizable lipids; one or more neutral lipids; and one or more PEGylated lipids.

In some embodiments, the one or more cationic lipids and the one or more ionizable lipids are present in the lipid particle in amount effective to produce a pKa apparent of from 6 to 10, such as from 6 to 8, as determined by a TNS pKa assay. The pKa apparent can be determined by a TNS pKa assay as defined by the formula below

$$pKa apparent = {pKa}_{0} + k * Q / T$$

were
pKa₀ represents the pKa of the ionizable lipid;
k represents an empirical constant determined by a TNS pKa assay
Q represents the mole % of the cationic lipid; and
T represents the mole % of the ionizable lipid.

In some embodiments, the one or more cationic lipids and the one or more ionizable lipids are present in the lipid particle in amounts that satisfy the expression below

$$6 < {pKa}_{0} + k * Q / T < 10$$

were
pKa₀ represents the pKa of the ionizable lipid;
k represents the empirical constant determined by a TNS pKa assay
Q represents the mole % of the cationic lipid; and
T represents the mole % of the ionizable lipid.

In some embodiments, the one or more cationic lipids and the one or more ionizable lipids are present in the lipid particle in amounts that satisfy the expression below

$$6 < {pKa}_{0} + k * Q / T < 8$$

were
pKa₀ represents the pKa of the ionizable lipid;
k represents the empirical constant determined by a TNS pKa assay
Q represents the mole % of the cationic lipid; and
T represents the mole % of the ionizable lipid.

In some embodiments, Q/T can be from greater than 0 to 1 (e.g., from greater than 0 to 0.25, from greater than 0 to 0.5, from greater than 0 to 0.75, from 0.25 to 0.5, from 0.25 to 0.75, from 0.25 to 1, from 0.5 to 0.75, from 0.5 to 1, or from 0.75 to 1).

In some embodiments, the one or more cationic lipids are present in the lipid particle in an amount of from greater than 0 mol % to 10 mol %, based on the total components forming the lipid particle. In certain embodiments (e.g., for local delivery), the one or more cationic lipids are present in the lipid particle in an amount of from 0.5 mol % to 5 mol %, based on the total components forming the lipid particle. In certain embodiments (e.g., for systemic delivery), the one or more cationic lipids are present in the lipid particle in an amount of from 4 mol % to 8 mol %, based on the total components forming the lipid particle.

In some embodiments, the one or more ionizable lipids are present in the lipid particle in an amount of from 20 mol % to 65 mol %, based on the total components forming the lipid particle.

In some embodiments, the one or more neutral lipids are present in the lipid particle in an amount of from 35 mol % to 80 mol %, based on the total components forming the lipid particle.

In some embodiments, the one or more PEGylated lipids are present in the lipid particle in an amount of from greater than 0 mol % to 5 mol %, based on the total components forming the lipid particle.

Also provided are pharmaceutical compositions comprising a lipid particle encapsulating an active agent, the lipid particle comprising: from greater than 0 mol % to 10 mol % (e.g., from 0.5 mol % to 10 mol %) one or more cationic lipids; from 20 mol % to 65 mol % one or more ionizable lipids, from 35 mol % to 80 mol % one or more neutral lipids; and from greater than 0 mol % to 5 mol % one or more PEGylated lipids.

In certain embodiments (e.g., for local delivery), the one or more cationic lipids are present in the lipid particle in an amount of from 0.5 mol % to 3.5 mol %, based on the total components forming the lipid particle. In certain embodiments (e.g., for systemic delivery), the one or more cationic lipids are present in the lipid particle in an amount of from 4 mol % to 8 mol %, based on the total components forming the lipid particle.

In some embodiments, the one or more ionizable lipids are present in the lipid particle in an amount of from 30 mol % to 50 mol %, based on the total components forming the lipid particle.

In some embodiments, the one or more neutral lipids are present in the lipid particle in an amount of from 30 mol % to 50 mol % of the total components forming the lipid particle.

In some embodiments, the one or more PEGylated lipids are present in the lipid particle in an amount of from 0.5 mol % to 3 mol % of the total components forming the lipid particle.

In some cases, the one or more ionizable lipids comprise a lipid headgroup comprising a tertiary amine. In some examples, the one or more ionizable lipids comprise N,N-dimethyl-2,3-dioleyloxypropylamine (DODMA), [(4-hydroxybutyl)azanediyl]di(hexane-6,1-diyl)bis(2-hexyldecanoate) (ALC-0315), 9-heptadecanyl 8-{(2-hydroxyethyl)[6-oxo-6-(undecyloxy)hexyl]amino}octanoate (SM-102), DLin-MC3-DMA, DLin-KC2-DMA, 1-(2,3-bis(((9Z,12Z)-octadeca-9,12-dien-1-yl)oxy)propyl)pyrrolidine (A066), or any combination thereof.

In some examples, the one or more neutral lipids comprise dipalmitoylphosphatidylcholine (DPPC), dioleoylphosphatidylethanolamine (DOPE), palmitoyloleoylphosphatidylcholine (POPC), egg phosphatidylcholine (EPC), distearoylphosphatidylcholine (DSPC), cholesterol, or any combination thereof.

In some examples, the one or more PEGylated lipids comprise a PEG-ditetradecylacetamide, a PEG-myristoyl diglyceride, a PEG-diacylglycerol, a PEG dialkyloxypropyl, a PEG-phospholipid, a PEG-ceramide, or any combinations thereof.

In some cases, the one or more cationic lipids comprise a lipid headgroup comprising a quaternary amine. In some examples, the one or more cationic lipids comprise DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA (2,3-dioleyloxy-N-[2-(spermine carboxamido)ethyl]-N,N-dimethyl-1-propanaminium trifluoroacetate), DORIE (N-[1-(2,3-dioleyloxypropyl)]-N,N-dimethyl-N-hydroxyethylammonium bromide), DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio) propane, DC-6-14:0,0-ditetradecanoyl-N-alpha.-trimethylammonioacetyl) diethanolamine chloride, CLIP 1: rac-[(2,3-dioctadecyloxypropyl)(2-hydroxyethyl)]-dimethylammonium chloride, CLIP6: rac-[2 (2,3-dihexadecyloxypropyloxymethyloxy)ethyl]-trimethylammonium, CLIP9: rac-[2 (2,3-dihexadecyloxypropyloxysuccinyloxy)ethyl]-trimethylammonium, oligofectamine, lipids described in U.S. Pat. No. 5,049,386, N-[1-(2,3-dioleyloxypropyl)]-N,N-dimethyl-N-hydroxyethylammonium bromide (DORIE), 2,3-dioleyloxy-N-[2-(spermine carboxamido)ethyl]-N,N-dimethyl-1-propanaminium trifluoroacetate (DOSPA), and the like as disclosed in International Publication Nos. WO91/16024 and WO97/019675; DLinDMA and the like as disclosed in International Publication No. WO2005/121348; and DLin-K-DMA and the like as disclosed in International Publication No. WO2009/086558; and (3R,4R)-3,4-bis((Z)-Hexadec-9-enyloxy)-1-methylpyrrolidine, and N-Methyl-N,N-bis(2-((Z)-octadec-6-enyloxy)ethyl) amine and the like as disclosed in International Publication No. WO2011/13636, or any combination thereof.

The lipid particles can have an average diameter of less than 1 micron, such as from from 50 nm to 750 nm, 50 nm to 250 nm, from 50 nm to 200 nm, from 50 nm to 150 nm, or from 50 nm to 100 nm. The lipid particles can have a polydispersity index (PDI) of less than 0.4.

The active agent encapsulated in the lipid particles can comprise any suitable active agent, such as a small molecule therapeutic agent, a diagnostic agent, a peptide, a protein, an antibody, or a nucleic acid. In certain embodiments, the active agent can comprise a nucleic acid, such as siRNA, mRNA, or any combination thereof.

The compositions described herein can be used to deliver one or more active agents to cells (e.g., in vivo, ex vivo, or in vitro). Accordingly, provided herein are method of delivering an active agent to a cell (e.g., in vivo, ex vivo, or in vitro) that comprise contacting the cell with a composition described herein. Also provided are methods for in vivo delivery of an active agent to a cell, said method comprising administering to a mammalian subject (e.g., a human) a composition described herein. In some embodiments, the administration can comprise systemic administration (e.g., intravenous injection or infusion).

Also provided are methods of systemically administering an active agent to a subject in need thereof. These methods can comprise intraveneously injecting a pharmaceutical composition comprising a lipid particle encapsulating the active agent, the lipid particle comprising: from 0.5 mol % to 8 mol % one or more cationic lipids; from 20 mol % to 65 mol % one or more ionizable lipids; from 35 mol % to 80 mol % one or more neutral lipids; and from greater than 0 mol % to 5 mol % one or more PEGylated lipids. In some cases, the active agent can comprise an anticancer agent.

Also provided are methods of administering an active agent to a liver of a subject. These methods can comprise intraveneously injecting a pharmaceutical composition comprising a lipid particle encapsulating the active agent, the lipid particle comprising: from 0.5 mol % to 3 mol % one or more cationic lipids; from 20 mol % to 65 mol % one or more ionizable lipids; from 35 mol % to 80 mol % one or more neutral lipids, and from greater than 0 mol % to 5 mol % one or more PEGylated lipids. In some cases, the active agent can comprise an anticancer agent, such as an active agent for the treatment of liver cancer.

Also provided are methods of administering an active agent to a solid tumor in a subject. These methods can comprise intraveneously injecting a pharmaceutical composition comprising a lipid particle encapsulating the active agent, the lipid particle comprising: from 3 mol % to 6 mol % one or more cationic lipids; from 20 mol % to 65 mol % one or more ionizable lipids; from 35 mol % to 80 mol % one or more neutral lipids; and from greater than 0 mol % to 5 mol % one or more PEGylated lipids. In some cases, the active agent can comprise an anticancer agent. These methods can selectively target the lipid particles to the tumor neovasculature.

Also provided are methods of administering an active agent to a lung of a subject. These methods can comprise intraveneously injecting a pharmaceutical composition comprising a lipid particle encapsulating the active agent, the lipid particle comprising: from 6 mol % to 10 mol % one or more cationic lipids; from 20 mol % to 65 mol % one or more ionizable lipids; from 35 mol % to 80 mol % one or more neutral lipids; and from greater than 0 mol % to 5 mol % one or more PEGylated lipids. In some cases, the active agent can comprise an anticancer agent, such as an active agent for the treatment of lung cancer.

Also provided are methods of locally administering an active agent to a subject in need thereof. These methods can comprises intramuscularly injecting a pharmaceutical composition comprising a lipid particle encapsulating the active agent, the lipid particle comprising: from greater than 0 mol % to 5 mol % one or more cationic lipids; from 20 mol % to 65 mol % one or more ionizable lipids; from 35 mol % to 80 mol % one or more neutral lipids; and from greater than 0 mol % to 5 mol % one or more PEGylated lipids. In some cases, the active agent can comprise a nucleic acid (e.g., siRNA, mRNA, or any combination thereof). In some cases, the active agent can comprise a vaccine.

### DESCRIPTION OF DRAWINGS

FIGS. 1A-1G are plots summarizing efforts to optimize cationic lipids (FIGS. 1A-1C), ionizable lipids (FIG. 1D), helper lipids (FIG. 1E), formulation procedures (FIG. 1F), and temperature control (FIG. 1G) for QTsome-based mRNA delivery. One-way ANOVA: * p<0.05, **: p<0.01, *** p<0.001.

FIGS. 2A-2C show the results of particle characterization of QTPlus-AM21. FIG. 2A shows the particle sizes for QTsome Original or QTPlus encapsulating AM21. FIG. 2B shows a cryo-EM image for QTPlus-AM21. FIG. 2C shows the 1% Agarose gel electrophoresis of QTPlus-AM21 with different nitrogen-to-phosphate (N/P) ratio.

FIGS. 3A-3B show miR-21 downstream gene regulation by 16-mer and 20-mer AM21 in free solution (FIG. 3A) and QTPlus (FIG. 3B).

FIGS. 4A-4C compare 16-mer and 20-mer AM21 in QTPlus-AM21 formulation (FIG. 4A) and tumor growth inhibition in vitro (FIGS. 4B and 4C).

FIGS. 5A-5C illustrate the ability of AM21 (FIG. 5B) and QTsome Original or QTPlus-AM21 (FIG. 5C) to regulate miR-21 downstream genes (FIG. 5A).

FIGS. 6A-6B demonstrate that QTPlus-AM21 showed superior tumor growth inhibition in vitro compared with free AM21.

FIG. 6C is a plot demonstrating that QTPlus-AM21 sensitized A549 to erlotinib chemotherapy.

FIG. 7 is a plot showing the acute cytotoxicity of QTPlus-AM21.

FIGS. 8A-8D show PTEN and EGFR expression in A549 tumor model treated with QTPlus-AM21 and erlotinib in vitro (FIGS. 8A and 8B) and in vivo (FIGS. 8C and 8D). One-way ANOVA: * p<0.05, **: p<0.01, *** p<0.001.

FIG. 9 is a plot showing the in vivo antitumor activity by QTsome Original-AM21 in A549 xenograft mouse model. One-way ANOVA: * p<0.05, **: p<0.01, *** p<0.001.

FIG. 10 is a plot showing the in vivo antitumor activity of QTPlus-AM21 and erlotinib in A549 xenograft mouse model. One-way ANOVA: * p<0.05, **: p<0.01, * p<0.001.

FIGS. 11A-11D show that QTPlus-AM21 regulates PD1/PD-L1 expression in THP-1 (FIG. 11A) and RAW264.7 (FIG. 11B) cell lines and induces macrophage polarization in THP-1 (FIG. 11C) and RAW264.7 (FIG. 11D) cell lines.

FIG. 12 shows that QTPlus-AM21 induces pro-inflammatory cytokines and chemokines in macrophage in vitro.

FIGS. 13A-13D show that QTPlus-AM21 regulates macrophages to inhibit tumor growth in vitro. FIG. 13A shows that RAW264.7 cells treated with QTPlus-AM21 inhibit wound-healing by MC38 colorectal cancer cells. FIG. 13B shows that QTPlus-AM21 regulates RAW264.7 cells to enhance apoptosis in MC38 cells. QTPlus-AM21 also induced macrophage proliferation (FIG. 13C) and polarization to M1 population (FIG. 13D). One-way ANOVA: * p<0.05, **: p<0.01, *** p<0.001.

FIG. 14 is a plot showing the in vivo antitumor activity of QTPlus-AM21 in MC38 syngeneic mouse model. One-way ANOVA: * p<0.05, **: p<0.01, *** p<0.001.

FIGS. 15A-15D show that QTPlus-AM21 increased CD45+ tumor-infiltrated immune cells (FIGS. 15A and 15B) and CD86+M1 population (FIGS. 15C and 15D) in the tumor microenvironment in vivo.

FIG. 16A shows QTPlus-AM21 induced CXCL10, IFNa, and TNFa in spleen tissues.

FIG. 16B shows that QTPlus and QTPlus regulated PD1/PD-L1 expression in mice tumors.

FIG. 16C shows that QTPlus and QTPlus regulated PD1/PD-L1 expression in spleen tissue.

FIG. 17 is a plot showing the in vivo antitumor activity of QTPlus-AM21 and atezolizumab in MC38 syngeneic mouse model. One-way ANOVA: * p<0.05, **: p<0.01, p<0.001.

FIG. 18 is a schematic illustration of a method for preparing QTsomes.

FIG. 19 is a plot showing firefly luciferase bioluminescence intensity measurements using Bright-Glo luciferase kit on HEK293T cells after treatment with 100 ng FFLuc mRNA using QTsome+ lipid nanoparticles.

FIG. 20 is a plot of firefly luciferase bioluminescence intensity measurements using Bright-Glo luciferase kit on HEK293T cells after treatment with 100 ng FFLuc mRNA using QTsome+ lipid nanoparticles.

FIGS. 21A and 21B show the results of particle size analysis of firefly luciferase mRNA-encapsulated QTsome+ lipid nanoparticles.

FIGS. 22A-22C show the particle sizes (FIG. 22A), zeta potential analysis (FIG. 22B) and beta-galactosidase activity analysis (FIG. 22C) of QTsome+ lipid nanoparticles encapsulating beta-galactosidase mRNA.

FIG. 23 is a plot showing the results obtained using firefly messenger RNA encapsulated QTsome+ lipid nanoparticles examined in A549 NSCLC cells.

FIG. 24 is a photo showing encapsulating efficiency determination by agarose gel electrophoresis.

FIG. 25 plots the apparent pKa of FFLuc mRNA-encapsulated QTsome+ lipid nanoparticles determined by TNS Assay.

FIGS. 26A-26B compare in vivo and in vitro delivery results obtaining using DSPC+1.5% DOTAP and DOPE+1.5% DOTAP.

FIG. 27 shows in vivo mRNA expression by IM injection and imaged by IVIS system.

FIG. 28 summarizes the results of the overall examination of FFLuc mRNA-encapsulated QTsome+ lipid nanoparticles in vivo through IM injection. Bioluminescence was examined by IVIS system.

FIG. 29 illustrates regulation and downstream effects of the Akt signaling pathway.

FIGS. 30A-30B are intensity weighting DLS diameter measurements of empty QTsome+ (FIG. 30A) and encapsulated QTsome+ (FIG. 30B).

FIG. 31 is a cryo-TEM micrograph of siAkt1 QTsome+. QTsome+ possesses a bilamellar structure with a smooth particle curvature.

FIGS. 32A-32B are plots showing the effect of siRNA-Akt on different targets of human liver cancer cell lines. Relative mRNA expression level was shown after transfection of siRNA-Scramble and siRNA-Akt at 25 nM on Hep3B, huh7, SNU387 and HepG2. Si-Akt encapsulated by lipofectamine 3000 was normalized against siRNA-Scramble encapsulated by lipofectamine 3000, while siRNA-Akt encapsulated by QTsome+ was normalized against siRNA-Scramble encapsulated by lipofectamine 3000.

FIG. 32C shows how protein expression was shown by western blot analysis to evaluate the level of different proteins on Hep3B and huh-7. β-actin served as an internal reference for both RT-PCR and western blot assay.

FIG. 32D shows how IL-6 and TNF-α release on human PBMCs were analyzed via ELISA.

FIG. 32 E shows how cell cycle was determined and analysis via flow cytometry.

FIGS. 33A-33B is a plot showing anti-tumor effect on male athymic BALB/C mice inoculated with huh7 xenograft model. Dose were given on day 1, day4, day 7 and day 10 via I.V. injection. The treatment starts when the tumor sizes were up to 100-150 mm³. The drug was given by I.V. injection every 3 days. The tumor size was suppressed efficiently by siRNA-Akt1 compared to normal saline group and siRNA ctrl group. Tumor size was calculated with equation:

$$Volume = length * \frac{{width}^{2}}{2} .$$

FIG. 33C is a plot showing the body weight loss observed during the course of treatment. There is slight body weight loss observed during the treatment in all the siRNA groups, about 5-10%. which indicates that the vehicle used to deliver siRNA has mild toxicity to some extent, but no more severe side effects such as dehydration, dizziness, asthenia or ulceration were observed.

FIG. 34 is a plot showing the average tumor size of C57BL6 mice bearing MC38 cells.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

“Aqueous solution” refers to a composition comprising in whole, or in part, water.

“Organic lipid solution” refers to a composition comprising in whole, or in part, an organic solvent having a lipid. In some embodiments, the organic lipid solution can comprise an alkanol, most preferably ethanol. In certain embodiments, the compositions described herein can be free of organic solvents, such as ethanol.

“Lipid” refers to a group of organic compounds that are esters of fatty acids and are characterized by being insoluble in water but soluble in many organic solvents, e.g, fats, oils, waxes, phospholipids, glycolipids, and steroids.

“Amphipathic lipid” comprises a lipid in which hydrophilic characteristics derive from the presence of polar or charged groups such as carbohydrates, phosphato, carboxylic, sulfato, amino, sulfhydryl, nitro, hydroxy and other like groups, and hydrophobic characteristics can be conferred by the inclusion of a polar groups that include, but are not limited to, long chain saturated and unsaturated aliphatic hydrocarbon groups and such groups substituted by one or more aromatic, cycloaliphatic or heterocyclic group(s). Examples include phospholipids, aminolipids and sphingolipids. Phospholipids include phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine or dilinoleoylphosphatidylcholine. Amphipathic lipids also can lack phosphorus, such as sphingolipid, glycosphingolipid families, diacylglycerols and b-acyloxyacids.

“Anionic lipid” is any lipid that is negatively charged at physiological pH, including phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, and other anionic modifying groups joined to neutral lipids.

“Cationic lipid” carry a net positive charge at a selective pH, such as physiological pH, including N,N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3-(N—(N′,N′-dimethylaminoethane)-carbamoyljcholesterol (“DC-Chol”) and N-(1,2-dimyristyloxyprop-3-yl)-N, N-dimethyl-N-hydroxyethyl ammonium bromide (“DMRIE”). Additionally, a number of commercial preparations of cationic lipids are available which can be used in the present invention. These include, for example, LIPOFECTIN®, LIPOFECT AMINE®, and TRANSFECTAM®.

### Pharmaceutical Compositions

Provided herein are pharmaceutical compositions that comprise a lipid particle encapsulating an active agent. The lipid particle can comprise one or more cationic lipids; one or more ionizable lipids; one or more neutral lipids; and one or more PEGylated lipids.

In some embodiments, the one or more cationic lipids and the one or more ionizable lipids are present in the lipid particle in amount effective to produce a pKa apparent of from 6 to 10, such as from 6 to 8, as determined by a TNS pKa assay. The pKa apparent can be determined by a TNS pKa assay as defined by the formula below

$$pKa apparent = {pKa}_{0} + k * Q / T$$

were
pKa₀ represents the pKa of the ionizable lipid;
k represents an empirical constant determined by a TNS pKa assay
Q represents the mole % of the cationic lipid; and
T represents the mole % of the ionizable lipid.

In some embodiments, the one or more cationic lipids and the one or more ionizable lipids are present in the lipid particle in amounts that satisfy the expression below

$$6 < {pKa}_{0} + k * Q / T < 10$$

were
pKa₀ represents the pKa of the ionizable lipid;
k represents the empirical constant determined by a TNS pKa assay
Q represents the mole % of the cationic lipid; and
T represents the mole % of the ionizable lipid.

In some embodiments, the one or more cationic lipids and the one or more ionizable lipids are present in the lipid particle in amounts that satisfy the expression below

$$6 < {pKa}_{0} + k * Q / T < 8$$

were
pKa₀ represents the pKa of the ionizable lipid;
k represents the empirical constant determined by a TNS pKa assay
Q represents the mole % of the cationic lipid; and
T represents the mole % of the ionizable lipid.

In some embodiments, Q/T can be from greater than 0 to 1 (e.g., from greater than 0 to 0.25, from greater than 0 to 0.5, from greater than 0 to 0.75, from 0.25 to 0.5, from 0.25 to 0.75, from 0.25 to 1, from 0.5 to 0.75, from 0.5 to 1, or from 0.75 to 1).

In some embodiments, the one or more cationic lipids are present in the lipid particle in an amount of from greater than 0 mol % to 10 mol %, based on the total components forming the lipid particle. In certain embodiments (e.g., for local delivery), the one or more cationic lipids are present in the lipid particle in an amount of from 0.5 mol % to 5 mol %, based on the total components forming the lipid particle. In certain embodiments (e.g., for systemic delivery), the one or more cationic lipids are present in the lipid particle in an amount of from 4 mol % to 8 mol %, based on the total components forming the lipid particle.

In some embodiments, the one or more ionizable lipids are present in the lipid particle in an amount of from 20 mol % to 65 mol %, based on the total components forming the lipid particle.

In some embodiments, the one or more neutral lipids are present in the lipid particle in an amount of from 35 mol % to 80 mol %, based on the total components forming the lipid particle.

In some embodiments, the one or more PEGylated lipids are present in the lipid particle in an amount of from greater than 0 mol % to 5 mol %, based on the total components forming the lipid particle.

Also provided are pharmaceutical compositions comprising a lipid particle encapsulating an active agent, the lipid particle comprising: from greater than 0 mol % to 10 mol % (e.g., from 0.5 mol % to 10 mol %) one or more cationic lipids; from 20 mol % to 65 mol % one or more ionizable lipids; from 35 mol % to 80 mol % one or more neutral lipids; and from greater than 0 mol % to 5 mol % one or more PEGylated lipids.

In certain embodiments (e.g., for local delivery), the one or more cationic lipids are present in the lipid particle in an amount of from 0.5 mol % to 3.5 mol %, based on the total components forming the lipid particle. In certain embodiments (e.g., for systemic delivery), the one or more cationic lipids are present in the lipid particle in an amount of from 4 mol % to 8 mol %, based on the total components forming the lipid particle.

In some embodiments, the one or more ionizable lipids are present in the lipid particle in an amount of from 30 mol % to 50 mol %, based on the total components forming the lipid particle.

In some embodiments, the one or more neutral lipids are present in the lipid particle in an amount of from 30 mol % to 50 mol % of the total components forming the lipid particle.

In some embodiments, the one or more PEGylated lipids are present in the lipid particle in an amount of from 0.5 mol % to 3 mol % of the total components forming the lipid particle.

The lipid particles can have an average diameter of less than 1 micron, such as from from 50 nm to 750 nm, 50 nm to 250 nm, from 50 nm to 200 nm, from 50 nm to 150 nm, or from 50 nm to 100 nm. The lipid particles can have a polydispersity index (PDI) of less than 0.4.

The components of these compositions are described in more detail below.

### Ionizable Lipids

As described above, the compositions described herein can comprise one or more ionizable lipids. An “ionizable lipid” is a lipid that carries a charge that is pH-dependent. The one or more ionizable lipids in the composition described herein can comprise ionizable cationic lipids which carry a positive or neutral charge depending on pH.

Generally, in lipid-based formulations for nucleic acid delivery, either a cationic lipid or an ionizable lipid is used to enable electrostatic interaction with the negatively charged cargo. A cationic lipid is typically defined as a lipid that carries a permanent positive charge(s) that typically comes from a quaternary amine. Examples of a cationic lipids include DOTAP, DOTMA, DDAB, and DODAC. In contrast, ionizable lipids include a chemical moiety, such as a tertiary amine(s), which is positively charged at acidic pH but becomes uncharged at neutral to basic pH. Ionizable lipids can have a pKa value in a biologically relevant range. However, the pKa value of such a lipid is highly dependent on the method used to measure it, resulting in up to 3 units of difference in numerical values for the same lipid. This has been documented in a recent article by Carrasco et al. Communications Biology volume 4, Article number: 956 (2021).

Examples of ionizable lipids are DODMA (N,N-dimethyl-2,3-dioleyloxypropylamine), DODAP, DLinDMA (1,2-dilinoleyloxy-3-dimethylaminopropane), DLinMC3DMA (dilinoleylmethyl-4-dimethylaminobutyrate), DLinKC2DMA (2-dilinoleyl-4-dimethylaminoethyl-[1,3]-dioxolane), ALC-0315 ([(4-hydroxybutyl)azanediyl]di(hexane-6,1-diyl)bis(2-hexyldecanoate), SM-102 (9-heptadecanyl 8-{(2-hydroxyethyl)[6-oxo-6-(undecyloxy)hexyl]amino}octanoate), Merck-32 (see e.g., WO 2012/018754), Acuitas-5 (see e.g., WO 2015/199952), KL-10 (see e.g., U.S. Patent Application Publication 2012/0295832), C12-200 (see e.g., Love, K T et al., PNAS, 107:1864 (2009)), and the like. Ionizable lipids also include those disclosed in U.S. Pat. Nos. 8,158,601, 9,593,077, 9,365,610, 9,567,296, 9,580,711, and 9,670,152, International Publication Nos. WO 2012/018754, WO 2015/199952, WO 2019/191780, and U.S. Patent Application Publication Nos. 2012/0295832, 2017/0190661 and 2017/0114010, each of which is incorporated herein by reference in its entirety.

In some embodiments, the one or more ionizable lipids can comprise a lipid headgroup comprising a tertiary amine. In certain embodiments, the one or more ionizable lipids can comprise N,N-dimethyl-2,3-dioleyloxypropylamine (DODMA), [(4-hydroxybutyl)azanediyl]di(hexane-6,1-diyl)bis(2-hexyldecanoate) (ALC-0315); 9-heptadecanyl 8-{(2-hydroxyethyl)[6-oxo-6-(undecyloxy)hexyl]amino}octanoate (SM-102), MC-3; KC-2; or any combination thereof.

In some embodiments, the one or more ionizable lipids comprise at least 20 mol % (e.g., at least 25 mol %, at least 30 mol %, at least 35 mol %, at least 40 mol %, at least 45 mol %, at least 50 mol %, at least 55 mol %, or at least 60 mol %) of the total components forming the lipid particle. In some embodiments, the one or more ionizable lipids comprise 65 mol % or less (e.g., 60 mol % or less, 55 mol % or less, 50 mol % or less, 45 mol % or less, 40 mol % or less, 35 mol % or less, 30 mol % or less, or 25 mol % or less) of the total components forming the lipid particle

The one or more ionizable lipids are present in the lipid particle in an amount ranging from any of the minimum values described above to any of the maximum values described above. For example, in some embodiments, the one or more ionizable lipids are present in the lipid particle in an amount of from 20 mol % to 65 mol % (e.g., from 30 mol % to 50 mol %) of the total components forming the lipid particle

### Neutral Lipids

As described above, the compositions described herein can comprise one or more neutral lipids.

Examples of neutral lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having Cio-C24 carbon chains, e.g., lauroyl, myristoyl, palmitoyl, stearoyl, or oleoyl.

Additional examples of neutral lipids include sterols such as cholesterol and derivatives thereof. Non-limiting examples of cholesterol derivatives include polar analogues such as 5a-cholestanol, 5a-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5a-cholestanone, and cholesteryl decanoate; and mixtures thereof. In preferred embodiments, the cholesterol derivative is a polar analogue such as cholesteryl-(4′-hydroxy)-butyl ether. Other examples of neutral lipids include nonphosphorous containing lipids such as, e.g. stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the one or more neutral lipids can comprise dipalmitoylphosphatidylcholine (DPPC), dioleoylphosphatidylethanolamine (DOPE), palmitoyloleoylphosphatidylcholine (POPC), egg phosphatidylcholine (EPC), distearoylphosphatidylcholine (DSPC), cholesterol, or any combination thereof.

In some embodiments, the one or more neutral lipids comprise at least 35 mol % (e.g., at least 40 mol %, at least 45 mol %, at least 50 mol %, at least 55 mol %, at least 60 mol %, at least 65 mol %, at least 70 mol %, or at least 75 mol %) of the total components forming the lipid particle. In some embodiments, the one or more neutral lipids comprise 80 mol % or less (e.g, 75 mol % or less, 70 mol % or less, 65 mol % or less, 60 mol % or less, 55 mol % or less, 50 mol % or less, 45 mol % or less, or 40 mol % or less) of the total components forming the lipid particle

The one or more neutral lipids are present in the lipid particle in an amount ranging from any of the minimum values described above to any of the maximum values described above. For example, in some embodiments, the one or more neutral lipids are present in the lipid particle in an amount of from 35 mol % to 80 mol % (30 mol % to 50 mol %) of the total components forming the lipid particle.

### PEGylated Lipids

As described above, the compositions described herein can comprise one or more PEGylated lipids. The one or more PEGylated lipids are useful in that they can reduce or prevent the aggregation of lipid particles.

PEG is a linear, water-soluble polymer of ethylene PEG repeating units with two terminal hydroxyl groups. PEGs are classified by their molecular weights; and include the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NEh), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S—NHS, HO-PEG-NH2).

Examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to glycerides forming a glycol, e.g., 1,2-dimyristoyl-sn-glycerol, methoxy-PEG glycol (PEG-DMG), PEG conjugated to ceramides, PEG conjugated to cholesterol, or a derivative thereof, and mixtures thereof. In some examples, the one or more PEGylated lipids can comprise, for example, a PEG-ditetradecylacetamide, a PEG-myristoyl diglyceride, a PEG-diacylglycerol, a PEG dialkyloxypropyl, a PEG-phospholipid, a PEG-ceramide, or any combinations thereof.

The PEG moiety of the PEG-lipid conjugates described herein may comprise an average molecular weight ranging from 550 Daltons to 10,000 Daltons. In certain instances, the PEG moiety has an average molecular weight of from 750 Daltons to 5,000 Daltons (e.g, from 1,000 Daltons to 5,000 Daltons, from 1,500 Daltons to 3,000 Daltons, from 750 Daltons to 3,000 Daltons, from 750 Daltons to 2,000 Daltons). In some embodiments, the PEG moiety has an average molecular weight of 2,000 Daltons or 750 Daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In one embodiment, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)—), urea (—NHC(O)NH—), disulphide (—S—S—), ether (—O—), succinyl (—(O)CCH2CH2C(O)—), succinamidyl (—NHC(O)CH2CH2C(O)NH—), ether, disulphide, as well as combinations thereof (such as a linker containing both a carbamate linker moiety and an amido linker moiety). In some embodiments, a carbamate linker is used to couple the PEG to the lipid. In other embodiments, an ester-containing linker moiety can be used to couple the PEG to the lipid. Suitable ester-containing linker moieties include, e.g., carbonate (—OC(O)O—), succinoyl, phosphate esters (—O—(O)POH—O—), sulfonate esters, and combinations thereof.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C12), myristoyl (Ci₄), palmitoyl (Ci6), stearoyl (Cis), and icosoyl (C20). In preferred embodiments, R¹ and R² are the same, i.e., R¹ and R² are both myristoyl (i.e., dimyristoyl), R¹ and R² are both stearoyl (i.e., distearoyl).

The term “dialkyloxyalkyl” or “DAA” includes a compound having 2 alkyl chains, R and R′, both of which have independently between 2 and 30 carbons. The alkyl groups can be saturated or have varying degrees of unsaturation.

Examples of PEG-DAA conjugates include PEG-didecyloxypropyl (C10), a PEG-dilauryloxypropyl (C12), a PEG-dimyristyloxypropyl (C14), a PEG-dipalmityloxypropyl (C16), and PEG-distearyloxypropyl (C18). In some of these embodiments, the PEG can have an average molecular weight of 750 or 2,000 Daltons. In certain embodiments, the terminal hydroxyl group of the PEG can be substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In some embodiments, the one or more PEGylated lipids comprise greater than 0 mol % (e.g., at least 0.5 mol %, at least 1 mol %, at least 1.5 mol %, at least 2 mol %, at least 2.5 mol %, at least 3 mol %, at least 3.5 mol %, at least 4 mol %, or at least 4.5 mol %) of the total components forming the lipid particle. In some embodiments, the one or more PEGylated lipids comprise 5 mol % or less (e.g., 4.5 mol % or less, 4 mol % or less, 3.5 mol % or less, 3 mol % or less, 2.5 mol % or less, 2 mol % or less, 1.5 mol % or less, 1 mol % or less, or 0.5 mol % or less) of the total components forming the lipid particle

The one or more PEGylated lipids are present in the lipid particle in an amount ranging from any of the minimum values described above to any of the maximum values described above. For example, in some embodiments, the one or more PEGylated lipids are present in the lipid particle in an amount of from greater than 0 mol % to 5 mol % of the total components forming the lipid particle.

### Cationic Lipids

As described above, the lipid blend described herein can comprise one or more cationic lipids (e.g., lipids bearing a quaternary ammonium moiety). Examples of cationic lipids include, for example, DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA (2,3-dioleyloxy-N-[2-(spermine carboxamido)ethyl]-N,N-dimethyl-1-propanaminium trifluoroacetate), DORIE (N-[1-(2,3-dioleyloxypropyl)]-N,N-dimethyl-N-hydroxyethylammonium bromide), DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio) propane, DC-6-14: O,O-ditetradecanoyl-N-alpha.-trimethylammonioacetyl) diethanolamine chloride, CLIP 1: rac-[(2,3-dioctadecyloxypropyl)(2-hydroxyethyl)]-dimethylammonium chloride, CLIP6: rac-[2 (2,3-dihexadecyloxypropyloxymethyloxy)ethyl]-trimethylammonium, CLIP9: rac-[2 (2,3-dihexadecyloxypropyloxysuccinyloxy)ethyl]-trimethylammonium, oligofectamine, lipids described in U.S. Pat. No. 5,049,386, N-[1-(2,3-dioleyloxypropyl)]-N,N-dimethyl-N-hydroxyethylammonium bromide (DORIE), 2,3-dioleyloxy-N-[2-(spermine carboxamido)ethyl]-N,N-dimethyl-1-propanaminium trifluoroacetate (DOSPA), and the like as disclosed in International Publication Nos. WO91/16024 and WO97/019675; DLinDMA and the like as disclosed in International Publication No. WO2005/121348; and DLin-K-DMA and the like as disclosed in International Publication No. WO2009/086558; and (3R,4R)-3,4-bis((Z)-Hexadec-9-enyloxy)-1-methylpyrrolidine, and N-Methyl-N,N-bis(2-((Z)-octadec-6-enyloxy)ethyl) amine and the like as disclosed in International Publication No. WO2011/13636.

In some embodiments, the one or more comprise lipids comprise greater than 0 mol % (e.g., at least 0.5 mol %, at least 1 mol %, at least 1.5 mol %, at least 2 mol %, at least 2.5 mol %, at least 3 mol %, at least 3.5 mol %, at least 4 mol %, at least 4.5 mol %, at least 5 mol %, at least 5.5 mol %, at least 6 mol %, at least 6.5 mol %, at least 7 mol %, at least 7.5 mol %, at least 8 mol %, at least 8.5 mol %, at least 9 mol %, or at least 9.5 mol %) of the total components forming the lipid particle. In some embodiments, the one or more PEGylated lipids comprise 10 mol % or less (e.g., 9.5 mol % or less, 9 mol % or less, 8.5 mol % or less, 8 mol % or less, 7.5 mol % or less, 7 mol % or less, 6.5 mol % or less, 6 mol % or less, 5.5 mol % or less, 5 mol % or less, 4.5 mol % or less, 4 mol % or less, 3.5 mol % or less, 3 mol % or less, 2.5 mol % or less, 2 mol % or less, 1.5 mol % or less, 1 mol % or less, or 0.5 mol % or less) of the total components forming the lipid particle

The one or more PEGylated lipids are present in the lipid particle in an amount ranging from any of the minimum values described above to any of the maximum values described above. For example, in some embodiments, the one or more PEGylated lipids are present in the lipid particle in an amount of from greater than 0 mol % to 10 mol % (e.g., from 0.5 mol % to 10 mol %, from 0.5 mol % to 8 mol %, from 0.5 mol % to 3.5 mol %, from 4 mol % to 8 mol %, from 0.5 mol % to 3 mol %, from 3 mol % to 6 mol %, or from 6 mol % to 10 mol %) of the total components forming the lipid particle.

### Active Agents

As used herein, an “active agent” refers to therapeutic agents, diagnostic agents, or prophylactic agents. As discussed herein, the therapeutic agents can be released from the disclosed compounds, compositions, and systems in a biologically active form.

It is further understood, that as used herein, the terms “therapeutic agents” refers to one or more therapeutic agents, active ingredients, or substances that can be used to treat a medical condition. Therapeutic agent includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments, vitamins and minerals such as essential amino acids, calcium, iron, potassium, zinc, vitamin B12, and the like; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; an antimicrobial agents (including antibiotics, antiviral agents, antiparasitic, and anti-fungal agents), anti-inflammatory agents (including steroids and non-steroidal anti-inflammatory agents), anti-coagulant agents, ophthalmic agents, gastrointestinal drugs, antiplatelet agents, and antiseptic agents, steroidal agent, anti-neoplastic agent, anti-cancer agent, antigen, antibody (e.g., cetuximab, anti-CD24 antibody, panitumumab and bevacizumab), birth control agent, progestational agent, anti-cholinergic, nutritional agent, analgesics and analgesic combinations such as acetaminophen, acetylsalicylic acid, and the like; anesthetics such as lidocaine, xylocaine, and the like, anorexics such as dexadrine, phendimetrazine tartrate, and the like; anti-epileptics, local and general anesthetics, hypnotics, sedatives, antipsychotic agents, neuroleptic agents, antidepressants such as isocarboxazid, amoxapine, and the like; anxiolytics, antagonists, neuron blocking agents, anticholinergic and cholinomimetic agents, antimuscarinic and muscarinic agents, antiparkinsonian agents, anti-Alzheimer's agents, antiadrenergics, antiarrhythmics, antihypertensive agents, hormones such as insulin, progestins, estrogens, corticoids, glucocorticoids, androgens, and the like; and nutrients, antiarthritics such as methylprednisolone, ibuprofen, and the like; antiasthmatics such as terbutaline sulfate, theophylline, ephedrine, and the like; anticonvulsants such as phenyloin sodium, diazepam, and the like; antiallergenics, antihistamines such as diphenhydramine HCl, chlorpheniramine maleate, and the like; antinauseants, antineoplastics, antipruritics, antipyretics, antispasmodics such as belladonna alkaloids, dicyclomine hydrochloride, and the like; cardiovascular agents such as prazosin HCl, nitroglycerin, propranolol HCl, hydralazine HCl, pancrelipase, succinic acid dehydrogenase, and the like; vasoactive agent, cardiovascular preparations (including calcium channel blockers, beta-blockers, beta-agonists and antiarrythmics), antihypertensives, diuretics such as furosemide, spironolactone, and the like; vasodilators; central nervous system stimulants; cough and cold preparations; decongestants; diagnostics; bone growth stimulants and bone resorption inhibitors; muscle relaxants; psychostimulants; sedatives; tranquilizers such as thorazine, diazepam, chlorpromazine HCl, reserpine, chlordiazepoxide HCl, and the like, antiulcer drugs such as rantidine HCl, cimetidine HCl, and the like; anti-asthmatic agents, anti-diarrheals, anti-obesity agents, anti-thrombotic agents, anti-tussive agents, anti-uricemic agents, anti-anginal agents, appetite suppressants, expectorants, hyperglycemic agents, hypoglycemic agents, thyroid and anti-thyroid agents, tissue growth agents, uterine relaxants, immunomodulator, including, for example, cytokines, interleukins, interferon, colony stimulating factor, tumor necrosis factor, and the like; immunosuppressants such as rapamycin, tacrolimus, and the like, immunological agent; antigens, factors, growth factors, amino acids, peptides and proteins and fragments thereof (whether naturally occurring, chemically synthesized or recombinantly produced) such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, antigens or antigenic polypeptides, enzymes, antibodies, monoclonal antibodies, and the like; and nucleic acid molecules (polymeric forms of two or more nucleotides, polynucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. In certain embodiments of the present disclosure, the agent to be delivered may be a mixture of active agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, molnupiravir, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of anticoagulant agents include, but are not limited to, heparin, warfarin, rivaroxaban, dabigatran, apixaban, edoxaban, enoxaparin, and fondaparinux.

Representative examples of antiplatelet agents include, but are not limited to, clopidogrel, ticagrelor, prasugrel, dipyridamole, dipyridamole/aspirin, ticlopidine, and eptifibatide.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

Other examples of active agents include chloroquine, hydrochloroquine, Pyridoxal phosphate, Vitamin D, and Vitamin C.

Representative examples of anticytokine or immunomodulatory agents, but are not limited to, tocilizumab, sarilumab, bevacizumab, fingolimod, imiquimod, and eculizumab.

Immunotherapeutic agent can include but are not limited to an anti-CD40 antibody, an anti-PDL I antibody (e.g., atezolizumab, durvalumab, or avelumab), an anti-PD1 antibody, an anti-CTLA4 antibody, programmed death protein 1 (PD-1) inhibitor or programmed death protein ligand 1 or 2 inhibitor include, (e.g., nivolumab (BMS), pembrolizumab (Merck), pidilizumab (CureTech/Teva), AMP-244 (Amplimmune/GSK), BMS-936559 (BMS), and MEDI4736 (Roche/Genentech)), or a combination thereof.

Representative examples of contraceptives include, but are not limited to, progestins, estrogens, or any combination thereof. For example, suitable progestins include, but are not limited to, natural and synthetic compounds having progestational activity, such as, for example, progesterone, chlormadinone acetate, norethindrone, cyproterone acetate, norethindrone acetate, desogestrel, levonorgestrel, drospirenone, trimegestone, norgestrel, norgestimate, norelgestromin, etonogestrel, gestodene, and other natural and/or synthetic gestagens. For example suitable estrogens include, but are not limited to, natural and synthetic compounds having estrogenic activity, such as, for example, estradiol (17β-estradiol), 17α-estradiol, estriol, estrone, and their esters, such as the acetate, sulfate, valerate or benzoate esters of these compounds, including, for example, estradiol 17β-cypionate, estradiol 17-propionate, estradiol 3-benzoate, and piperazine estrone sulfate; ethinyl estradiol; conjugated estrogens (natural and synthetic); mestranol; agonistic anti-estrogens, and selective estrogen receptor modulators. Other examples of contraceptives include gonodotropin releasing hormone (GnRh) or anologs thereof such as deslorelin, avorelin, leuprolide, triptorelin, nafarelin, goserelin, buserelin, and fertirelin.

The term “steroid” refers to compounds belonging to or related to the following illustrative families of compounds: corticosteroids, mineralicosteroids, and sex steroids (including, for example, potentially androgenic or estrogenic or anti-androgenic and anti-estrogenic molecules). Included among these are, for example, prednisone, prednisolone, methyl-prednisolone, triamcinolone, fluocinolone, aldosterone, spironolactone, danazol (otherwise known as OPTINA), and others. In some embodiments, the therapeutic agent may comprise a steroid.

Exemplary cancer drugs or anti-cancer agents can include, but are not limited to, antimetabolite anti-cancer agents and antimitotic anti-cancer agents, and combinations thereof. Various antimetabolite and antimitotic anti-cancer agents, including single such agents or combinations of such agents, may be employed in the methods and compositions described herein.

Antimetabolic anti-cancer agents typically structurally resemble natural metabolites, which are involved in normal metabolic processes of cancer cells such as the synthesis of nucleic acids and proteins. The antimetabolites, however, differ enough from the natural metabolites such that they interfere with the metabolic processes of cancer cells. In the cell, antimetabolites are mistaken for the metabolites they resemble, and are processed by the cell in a manner analogous to the normal compounds. The presence of the “decoy” metabolites prevents the cells from carrying out vital functions and the cells are unable to grow and survive. For example, antimetabolites may exert cytotoxic activity by substituting these fraudulent nucleotides into cellular DNA, thereby disrupting cellular division, or by inhibition of critical cellular enzymes, which prevents replication of DNA.

In one aspect, therefore, the antimetabolite anti-cancer agent is a nucleotide or a nucleotide analog. In certain aspects, for example, the antimetabolite agent may comprise purine (e.g., guanine or adenosine) or analogs thereof, or pyrimidine (cytidine or thymidine) or analogs thereof, with or without an attached sugar moiety.

Suitable antimetabolite anti-cancer agents for use in the present disclosure may be generally classified according to the metabolic process they affect, and can include, but are not limited to, analogues and derivatives of folic acid, pyrimidines, purines, and cytidine. Thus, in one aspect, the antimetabolite agent(s) is selected from the group consisting of cytidine analogs, folic acid analogs, purine analogs, pyrimidine analogs, and combinations thereof.

In one particular aspect, for example, the antimetabolite agent is a cytidine analog. According to this aspect, for example, the cytidine analog may be selected from the group consisting of cytarabine (cytosine arabinodside), azacitidine (5-azacytidine), and salts, analogs, and derivatives thereof.

In another particular aspect, for example, the antimetabolite agent is a folic acid analog. Folic acid analogs or antifolates generally function by inhibiting dihydrofolate reductase (DHFR), an enzyme involved in the formation of nucleotides; when this enzyme is blocked, nucleotides are not formed, disrupting DNA replication and cell division. According to certain aspects, for example, the folic acid analog may be selected from the group consisting of denopterin, methotrexate (amethopterin), pemetrexed, pteropterin, raltitrexed, trimetrexate, and salts, analogs, and derivatives thereof.

In another particular aspect, for example, the antimetabolite agent is a purine analog. Purine-based antimetabolite agents function by inhibiting DNA synthesis, for example, by interfering with the production of purine containing nucleotides, adenine and guanine which halts DNA synthesis and thereby cell division. Purine analogs can also be incorporated into the DNA molecule itself during DNA synthesis, which can interfere with cell division. According to certain aspects, for example, the purine analog may be selected from the group consisting of acyclovir, allopurinol, 2-aminoadenosine, arabinosyl adenine (ara-A), azacitidine, azathiprine, 8-aza-adenosine, 8-fluoro-adenosine, 8-methoxy-adenosine, 8-oxo-adenosine, cladribine, deoxycoformycin, fludarabine, gancylovir, 8-aza-guanosine, 8-fluoro-guanosine, 8-methoxy-guanosine, 8-oxo-guanosine, guanosine diphosphate, guanosine diphosphate-beta-L-2-aminofucose, guanosine diphosphate-D-arabinose, guanosine diphosphate-2-fluorofucose, guanosine diphosphate fucose, mercaptopurine (6-MP), pentostatin, thiamiprine, thioguanine (6-TG), and salts, analogs, and derivatives thereof.

In yet another particular aspect, for example, the antimetabolite agent is a pyrimidine analog. Similar to the purine analogs discussed above, pyrimidine-based antimetabolite agents block the synthesis of pyrimidine-containing nucleotides (cytosine and thymine in DNA; cytosine and uracil in RNA). By acting as “decoys,” the pyrimidine-based compounds can prevent the production of nucleotides, and/or can be incorporated into a growing DNA chain and lead to its termination. According to certain aspects, for example, the pyrimidine analog may be selected from the group consisting of ancitabine, azacitidine, 6-azauridine, bromouracil (e.g., 5-bromouracil), capecitabine, carmofur, chlorouracil (e.g. 5-chlorouracil), cytarabine (cytosine arabinoside), cytosine, dideoxyuridine, 3′-azido-3′-deoxythymidine, 3′-dideoxycytidin-2′-ene, 3′-deoxy-3′-deoxythymidin-2′-ene, dihydrouracil, doxifluridine, enocitabine, floxuridine, 5-fluorocytosine, 2-fluorodeoxycytidine, 3-fluoro-3′-deoxythymidine, fluorouracil (e.g., 5-fluorouracil (also known as 5-FU), gemcitabine, 5-methylcytosine, 5-propynylcytosine, 5-propynylthymine, 5-propynyluracil, thymine, uracil, uridine, and salts, analogs, and derivatives thereof. In one aspect, the pyrimidine analog is other than 5-fluorouracil. In another aspect, the pyrimidine analog is gemcitabine or a salt thereof.

In certain aspects, the antimetabolite agent is selected from the group consisting of 5-fluorouracil, capecitabine, 6-mercaptopurine, methotrexate, gemcitabine, cytarabine, fludarabine, pemetrexed, and salts, analogs, derivatives, and combinations thereof. In other aspects, the antimetabolite agent is selected from the group consisting of capecitabine, 6-mercaptopurine, methotrexate, gemcitabine, cytarabine, fludarabine, pemetrexed, and salts, analogs, derivatives, and combinations thereof. In one particular aspect, the antimetabolite agent is other than 5-fluorouracil. In a particularly preferred aspect, the antimetabolite agent is gemcitabine or a salt or thereof (e.g., gemcitabine HCl (Gemzar®)).

Other antimetabolite anti-cancer agents may be selected from, but are not limited to, the group consisting of acanthifolic acid, aminothiadiazole, brequinar sodium, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, Wellcome EHNA, Merck & Co. EX-015, fazarabine, fludarabine phosphate, N-(2′-furanidyl)-5-fluorouracil, Daiichi Seiyaku FO-152, 5-FU-fibrinogen, isopropyl pyrrolizine, Lilly LY-188011; Lilly LY-264618, methobenzaprim, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, tiazofurin, Erbamont TIF, tyrosine kinase inhibitors, Taiho UFT and uricytin, among others.

In one aspect, the antimitotic anti-cancer agent is a microtubule inhibitor or a microtubule stabilizer. In general, microtubule stabilizers, such as taxanes and epothilones, bind to the interior surface of the beta-microtubule chain and enhance microtubule assembly by promoting the nucleation and elongation phases of the polymerization reaction and by reducing the critical tubulin subunit concentration required for microtubules to assemble. Unlike mictrotubule inhibitors, such as the vinca alkaloids, which prevent microtubule assembly, the microtubule stabilizers, such as taxanes, decrease the lag time and dramatically shift the dynamic equilibrium between tubulin dimers and microtubule polymers towards polymerization. In one aspect, therefore, the microtubule stabilizer is a taxane or an epothilone. In another aspect, the microtubule inhibitor is a vinca alkaloid.

In some embodiments, the anti-cancer agent may comprise a taxane or derivative or analog thereof. The taxane may be a naturally derived compound or a related form, or may be a chemically synthesized compound or a derivative thereof, with antineoplastic properties. The taxanes are a family of terpenes, including, but not limited to paclitaxel (Taxol®) and docetaxel (Taxotere®), which are derived primarily from the Pacific yew tree, Taxus brevifolia, and which have activity against certain tumors, particularly breast and ovarian tumors. In one aspect, the taxane is docetaxel or paclitaxel. Paclitaxel is a preferred taxane and is considered an antimitotic agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

Also included are a variety of known taxane derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; deoxygenated paclitaxel compounds such as those described in U.S. Pat. No. 5,440,056; and taxol derivatives described in U.S. Pat. No. 5,415,869. As noted above, it further includes prodrugs of paclitaxel including, but not limited to, those described in WO 98/58927; WO 98/13059; and U.S. Pat. No. 5,824,701. The taxane may also be a taxane conjugate such as, for example, paclitaxel-PEG, paclitaxel-dextran, paclitaxel-xylose, docetaxel-PEG, docetaxel-dextran, docetaxel-xylose, and the like. Other derivatives are mentioned in “Synthesis and Anticancer Activity of Taxol Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elsevier, Amsterdam 1986), among other references. Each of these references is hereby incorporated by reference herein in its entirety.

Various taxanes may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267) (each of which is hereby incorporated by reference herein in its entirety), or obtained from a variety of commercial sources, including for example, Sigma-Aldrich Co., St. Louis, Mo.

Alternatively, the antimitotic anti-cancer agent can be a microtubule inhibitor; in one preferred aspect, the microtubule inhibitor is a vinca alkaloid. In general, the vinca alkaloids are mitotic spindle poisons. The vinca alkaloid agents act during mitosis when chromosomes are split and begin to migrate along the tubules of the mitosis spindle towards one of its poles, prior to cell separation. Under the action of these spindle poisons, the spindle becomes disorganized by the dispersion of chromosomes during mitosis, affecting cellular reproduction. According to certain aspects, for example, the vinca alkaloid is selected from the group consisting of vinblastine, vincristine, vindesine, vinorelbine, and salts, analogs, and derivatives thereof.

The antimitotic anti-cancer agent can also be an epothilone. In general, members of the epothilone class of compounds stabilize microtubule function according to mechanisms similar to those of the taxanes. Epothilones can also cause cell cycle arrest at the G2-M transition phase, leading to cytotoxicity and eventually apoptosis. Suitable epithiolones include epothilone A, epothilone B, epothilone C, epothilone D, epothilone E, and epothilone F, and salts, analogs, and derivatives thereof. One particular epothilone analog is an epothilone B analog, ixabepilone (Ixempra™).

In certain aspects, the antimitotic anti-cancer agent is selected from the group consisting of taxanes, epothilones, vinca alkaloids, and salts and combinations thereof. Thus, for example, in one aspect the antimitotic agent is a taxane. More preferably in this aspect the antimitotic agent is paclitaxel or docetaxel, still more preferably paclitaxel. In another aspect, the antimitotic agent is an epothilone (e.g., an epothilone B analog) In another aspect, the antimitotic agent is a vinca alkaloid.

Examples of cancer drugs that may be used in the present disclosure include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib.

Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine, purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, histrelin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations, progestins such as hydroxyprogesterone caproate, medroxyprogesterone acetate and megestrol acetate; estrogens such as dietbylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade-names of these and additional examples of cancer drugs, and their methods of use including dosing and administration regimens, will be known to a person versed in the art.

In some aspects, the anti-cancer agent may comprise a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof.

According to these aspects, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate aspect of the invention.

Anti-neoplastic agent can be selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Interferon Alfa-2b, Recombinant, Intron A (Recombinant Interferon Alfa-2b), Iodine I 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritale (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and Iodine I 131 Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

Growth factors useful as therapeutic agents include, but are not limited to, transforming growth factor-α (“TGF-α”), transforming growth factors (“TGF-β”), platelet-derived growth factors (“PDGF”), fibroblast growth factors (“FGF”), including FGF acidic isoforms 1 and 2, FGF basic form 2 and FGF 4, 8, 9 and 10, nerve growth factors (“NGF”) including NGF 2.5 s, NGF 7.0 s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, and bone derived growth factors and mixtures thereof.

Immunoglobulins useful in the present disclosure include, but are not limited to, IgG, IgA, IgM, IgD, IgE, and mixtures thereof. Some preferred growth factors include VEGF (vascular endothelial growth factor), NGFs (nerve growth factors), PDGF-AA, PDGF-BB, PDGF-AB, FGFb, FGFa, and BGF.

Other molecules useful as anti-cancer agents include but are not limited to growth hormones, leptin, leukemia inhibitory factor (LIF), tumor necrosis factor alpha and beta, endostatin, thrombospondin, osteogenic protein-1, bone morphogenetic proteins 2 and 7, osteonectin, somatomedin-like peptide, osteocalcin.

Tumor antigens can be based on specific mutations (neoepitopes) and those expressed by cancer-germline genes (antigens common to tumors found in multiple patients, referred to herein as “traditional cancer antigens” or “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

Diagnostic agents include gases; metals; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

In some embodiments, the active agent can comprise a vaccine or a component thereof. Vaccines may comprise isolated proteins or peptides, inactivated organisms and viruses, dead organisms and viruses, genetically altered organisms or viruses, cell extracts, and RNA encoding at least one antigenic polypeptide or an immunogenic fragment thereof (e.g., an immunogenic fragment capable of inducing an immune response to the antigenic polypeptide). Active agents may be combined with interleukins, interferon, cytokines, and adjuvants such as cholera toxin, alum, Freund's adjuvant, etc. Prophylactic agents can include infection agents such as antigens of such bacterial organisms as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), coronaviruses (e.g., MERS-COV, SARS-COV, SARS-COV2, HCoV-OC43, HCoV-229E, HCoV-NL63, HCoV-NL, HCoV-NH, HCoV-HKU1), poxviruses (e.g., smallpox, monkeypox), African swine virus, influenza A and B, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In certain embodiments, the active agent is a polynucleotide. Polynucleotides or oligonucleotides that can be introduced according to the methods herein include DNA, cDNA, and RNA sequences of all types. For example, the polynucleotide can be double stranded DNA, single-stranded DNA, complexed DNA, encapsulated DNA, naked RNA, encapsulated RNA, messenger RNA (mRNA), IRNA, short interfering RNA (siRNA), double stranded RNA (dsRNA), micro-RNA (miRNA), antisense RNA (asRNA), self-amplify mRNA (saRNA), guide RNA (gRNA), CRNA and combinations thereof. The polynucleotides can also be DNA constructs, such as expression vectors, expression vectors encoding a desired gene product (e.g., a gene product homologous or heterologous to the subject into which it is to be introduced), and the like.

A nucleic acid (NA, e.g., a polynucleotide or oligonucleotide) encoding a peptide may be used to produce an antigenic peptide in vitro. The NA may be, e.g., DNA, cDNA, PNA, CNA, RNA, either single- and/or double-stranded, or native or stabilized forms of polynucleotides, such as e.g., polynucleotides with a phosphorothioate backbone, or combinations thereof and it may or may not contain introns so long as it codes for the peptide. In one embodiment in vitro translation is used to produce the peptide. Many exemplary systems exist that one skilled in the art could utilize.

In some embodiments, the active agent can comprise a mRNA or an expression vector capable of expressing a polypeptide. Expression vectors for different cell types are well known in the art and can be selected without undue experimentation. Generally, the DNA is inserted into an expression vector, such as a plasmid, in proper orientation and correct reading frame for expression, if necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) MOLECULAR CLONING, A LABORATORY MANUAL, Cold Spring Harbor Laboratory, NY).

The term “nucleic acid encoding a polypeptide” encompasses a NA that includes only coding sequences for the polypeptide as well as a NA that includes additional coding and/or non-coding sequences. NA can be in the form of RNA or in the form of DNA. DNA includes cDNA, genomic DNA, and synthetic DNA; and can be double-stranded or single-stranded, and if single stranded can be the coding strand or non-coding (anti-sense) strand.

The NA may comprise the coding sequence for the peptide, either an antibody or an antigen, fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a pre-protein and can have the leader sequence cleaved by the host cell to form the mature form of the polypeptide.

A NA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest. An oligomer containing a NA sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors may be used to amplify and express DNA encoding antibodies or antigenic peptides. Recombinant expression vectors are replicable DNA constructs having synthetic or cDNA-derived DNA fragments operatively linked to suitable transcriptional or translational regulator elements derived from mammalian, microbial, viral or insect genes. A transcriptional unit generally comprises an assembly of a genetic element or elements having a regulatory role in gene expression, for example, transcriptional promoters or enhancers, a structural or coding sequence which is transcribed into mRNA and translated into protein, and appropriate transcription and translation initiation and termination sequences, as described in detail herein. Such regulatory elements can include an operator sequence to control transcription. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

“Ribonucleic acid” or “RNA” refers to a polymer containing at least two ribonucleotides. “Ribonucleotides” contain a sugar ribose, a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

RNA may be in the form of oligonucleotide RNA, tRNA (transfer RNA), snRNA (small nuclear RNA), IRNA (ribosomal RNA), mRNA (messenger RNA), antisense RNA, siRNA (small interfering RNA), self-replicating RNA, ribozymes, chimeric sequences, or derivatives of these groups.

The RNA can include (in addition to any 5′ cap structure) one or more nucleotides having a modified nucleobase, including m5C (5-methylcytidine), m5U (5-methyluridine), m6A (N6-methyladenosine), s2U (2-thiouridine), Um (2′-O-methyluridine), mlA (1-methyladenosine); m2A (2-methyladenosine); Am (2′-O-methyladenosine), ms2m6A (2-methylthio-N6-methyladenosine); i6A (N6-isopentenyladenosine); ms216A (2-methylthio-N6isopentenyladenosine); io6A (N6-(cis-hydroxyisopentenyl) adenosine); ms2106A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m616A (N6-methyl-N6-threonylcarbamoyladenosine); hn6A (N6.-hydroxynorvalylcarbamoyl adenosine); ms2hn6A (2-methylthio-N6-hydroxynorvalyl carbamoyladenosine); Ar(p) (2′-O-ribosyladenosine(phosphate)); I (inosine); ml 1 (1-methylinosine); m′lm (1,2′-O-dimethylinosine); m3C (3-methylcytidine); Cm (2T-O-methylcytidine); s2C (2-thiocytidine); ac4C (N4-acetylcytidine); f5C (5-fonnylcytidine); m5Cm (5,2-O-dimethylcytidine); ac4Cm (N4acetyl2TOmethylcytidine); k2C (lysidine); mlG (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr (p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); mSUm (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcmSU (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnmSU (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethylaminomethyl-2-L-O-methyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); rn62Am (N6,N6,O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); mlGm (1,2′-O-dimethylguanosine); m′Am (1, 2-O-dimethyl adenosine) irinomethyluridine); tm5s2U (S-taurinomethyl-2-thiouridine)); imG-14 (4-demethyl guanosine); imG2 (isoguanosine); or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-C6)-alkenyluracil, 5-(C2-C6)-alkynyluracil, 5-(hydroxymethyl) uracil, 5-chlorouracil, 5-fluorouracil, 5-bromouracil, 5-hydroxy cytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, or an abasic nucleotide.

The RNA may optionally comprise one or more UNA molecules, e.g., as disclosed in U.S. Pat. Nos. 8,314,227, 9,051,570, 9,303,260, 9,297,009, and 9,340,789, and U.S. Patent Publication No. 2016/0168567, incorporated herein in their entirety.

The RNA or self-replicating RNA can include one or more modified pyrimidine nucleobases, such as pseudouridine and/or 5-methylcytosine residues. The RNA may include a 5′ cap comprising a 7′-methylguanosine, and the first 1, 2 or 3 5′ ribonucleotides may be methylated at the 2′ position of the ribose. The RNA can contain a 5′ trinucleotide cap structure as described by Tanis, et al., U.S. application Ser. No. 15/788,742, filed Oct. 19, 2017, herein incorporated by reference in its entirety.

Natural RNA have a phosphate backbone, RNA as described herein may contain other types of backbones and bases including peptide nucleic acids, phosphothionates, phosphoramidate, phosphorothioate, and/or methylphosphonate linkages.

“Antisense” is a polynucleotide that interferes with the function of DNA and/or RNA. This may result in suppression of expression.

“Gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, and the like) of the full-length polypeptide or fragment thereof are retained.

In some embodiments, the RNA (e.g., mRNA) may be used to induce a balanced immune response against an infection agent. In some embodiments, the RNA (e.g., mRNA) may be used to induce a balanced immune response against a a Metapneumovirus such as human Metapneumovirus (hMPV), parainfluenza viruses such as human parainfluenza viruses (hPIV) types 1, 2, and 3 (hPIV1, hPIV2 and hPIV3, respectively), respiratory syncytial virus (RSV), measles virus (MeV), coronaviruses (e.g., MERS-COV, SARS-COV, SARS-COV2, HCoV-OC43, HCoV-229E, HCoV-NL63, HCOV-NL, HCoV-NH, HCOV-HKU1), poxviruses (e.g., smallpox, monkeypox), African swine virus, influenza A and B, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like.

In some embodiments, the RNA (e.g., mRNA) may be used to induce a balanced immune response against a respiratory virus. The term “respiratory virus” refers herein to a virus causing a respiratory disease. For example, negative-sense, single-stranded RNA virus of the family Paramyxoviridae such as human Metapneumovirus (hMPV), human parainfluenza viruses (hPIV) types 1, 2, and 3 (hPIV1, hPIV2 and hPIV3, respectively), RSV, and Measles virus (MeV). Another example of respiratory viruses are coronaviruses. Coronaviruses are enveloped viruses with a positive-sense single-stranded RNA genome and with a nucleocapsid of helical symmetry. Coronaviruses are species of virus belonging to the subfamily Coronavirinae in the family Coronaviridae, in the order Nidovirales.

Representative examples of betacoronaviruses include, but are not limited to an embecovirus 1 (e.g., Betacoronavirus 1, Human coronavirus OC43, China Rattus coronavirus HKU24, Human coronavirus HKU1, Murine coronavirus), a hibecovirus (e.g., Bat Hp-betacoronavirus Zhejiang2013), a merbecovirus (e.g., Hedgehog coronavirus 1, Middle East respiratory syndrome-related coronavirus (MERS-COV), Pipistrellus bat coronavirus HKU5, Tylonycteris bat coronavirus HKU4), a nobecovirus (e.g., Rousettus bat coronavirus GCCDC1, Rousettus bat coronavirus HKU9), a sarbecovirus (e.g., severe acute respiratory syndrome coronavirus (SARS-COV), severe acute respiratory syndrome coronavirus 2 (SARS-COV-2).

Representative examples of gammacoronaviruses include, but are not limited to, a cegacovirus (e.g., Beluga whale coronavirus SQ1) and an Igacovirus (e.g., Avian coronavirus (IBV)).

Representative examples of deltacoronaviruses include, but are not limited to, an andecovirus (e.g., Wigeon coronavirus HKU20), a buldecovirus (e.g., Bulbul coronavirus HKU11, Porcine coronavirus HKU15 (PorCoV HKU15), Munia coronavirus HKU13, White-eye coronavirus HKU16), a herdecovirus (e.g., Night heron coronavirus HKU19), and a moordecovirus (e.g., Common moorhen coronavirus HKU21).

In some embodiments, the coronavirus is a human coronavirus. Representative examples of human coronaviruses include, but are not limited to, human coronavirus 229E (HCoV-229E), human coronavirus OC43 (HCoV-OC43), human coronavirus HKU1 (HCoV-HKU1), Human coronavirus NL63 (HCoV-NL63), severe acute respiratory syndrome coronavirus (SARS-COV), severe acute respiratory syndrome coronavirus 2 (SARS-COV-2), and Middle East respiratory syndrome-related coronavirus (MERS-COV).

In some embodiments, the RNA (e.g., mRNA) polynucleotide having an open reading frame encoding at least one (e.g., at least 2, 3, 4 or 5) human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus I (HSV1) antigenic polypeptide, herpes simplex virus 2 (HSV2) antigenic polypeptide, poxvirus (e.g., smallpox, monkeypox) antigenic polypeptide, African swine virus antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, or coronaviruses (e.g., MERS-COV, SARS-COV, SARS-CoV2, HCoV-OC43, HCoV-229E, HCoV-NL63, HCoV-NL, HCoV-NH, HCoV-HKU1) antigenic polypeptide. Herein, use of the term “antigenic polypeptide” encompasses immunogenic fragments of the antigenic polypeptide (an immunogenic fragment that induces (or is capable of inducing) an immune response human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), varicella-zoster, influenza virus, herpes simplex virus 1 (HSV1), herpes simplex virus 2 (HSV2), poxvirus (e.g., smallpox, monkeypox), African swine virus, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, or coronaviruses (e.g., MERS-COV, SARS-COV, SARS-COV2, HCoV-OC43, HCoV-229E, HCoV-NL63, HCoV-NL, HCoV-NH, HCOV-HKU1), or any combination thereof.

### Methods of Use

These compositions can be prepared as described herein or elsewhere, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer, intratracheal or intranasal), oral, or parenteral. Parenteral administration includes intravenous, intraarterial, subcutaneous, intraperitoneal intramuscular or injection or infusion; or intracranial, (e.g., intrathecal or intraventricular, administration). Parenteral administration can be in the form of a single bolus dose, or may be, for example, by a continuous perfusion pump. In some embodiments, the compounds provided herein, or a pharmaceutically acceptable salt thereof, are suitable for parenteral administration. In some embodiments, the compounds provided herein are suitable for intravenous administration. In some embodiments, the compounds provided herein are suitable for oral administration. In some embodiments, the compounds provided herein are suitable for topical administration.

Pharmaceutical compositions and formulations for topical administration may include, but are not limited to, transdermal patches, ointments, lotions, creams, gels, drops, suppositories, sprays, liquids and powders. Conventional pharmaceutical carriers, aqueous, powder or oily bases, thickeners and the like may be necessary or desirable. In some embodiments, the pharmaceutical compositions provided herein are suitable for parenteral administration. In some embodiments, the pharmaceutical compositions provided herein are suitable for intravenous administration. In some embodiments, the pharmaceutical compositions provided herein are suitable for oral administration. In some embodiments, the pharmaceutical compositions provided herein are suitable for topical administration.

The compositions described herein can be used to deliver one or more active agents to cells (e.g., in vivo, ex vivo, or in vitro). Accordingly, provided herein are method of delivering an active agent to a cell (e.g., in vivo, ex vivo, or in vitro) that comprise contacting the cell with a composition described herein. Also provided are methods for in vivo delivery of an active agent to a cell, said method comprising administering to a mammalian subject (e.g., a human) a composition described herein. In some embodiments, the administration can comprise systemic administration (e.g., intravenous injection or infusion).

Also provided are methods of systemically administering an active agent to a subject in need thereof. These methods can comprise intraveneously injecting a pharmaceutical composition comprising a lipid particle encapsulating the active agent, the lipid particle comprising: from 0.5 mol % to 8 mol % one or more cationic lipids; from 20 mol % to 65 mol % one or more ionizable lipids; from 35 mol % to 80 mol % one or more neutral lipids; and from greater than 0 mol % to 5 mol % one or more PEGylated lipids. In some cases, the active agent can comprise an anticancer agent.

Also provided are methods of administering an active agent to a liver of a subject. These methods can comprise intraveneously injecting a pharmaceutical composition comprising a lipid particle encapsulating the active agent, the lipid particle comprising: from 0.5 mol % to 3 mol % one or more cationic lipids; from 20 mol % to 65 mol % one or more ionizable lipids; from 35 mol % to 80 mol % one or more neutral lipids; and from greater than 0 mol % to 5 mol % one or more PEGylated lipids. In some cases, the active agent can comprise an anticancer agent, such as an active agent for the treatment of liver cancer.

Also provided are methods of administering an active agent to a solid tumor in a subject. These methods can comprise intraveneously injecting a pharmaceutical composition comprising a lipid particle encapsulating the active agent, the lipid particle comprising: from 3 mol % to 6 mol % one or more cationic lipids; from 20 mol % to 65 mol % one or more ionizable lipids; from 35 mol % to 80 mol % one or more neutral lipids; and from greater than 0 mol % to 5 mol % one or more PEGylated lipids. In some cases, the active agent can comprise an anticancer agent. These methods can selectively target the lipid particles to the tumor neovasculature.

Also provided are methods of administering an active agent to a lung of a subject. These methods can comprise intraveneously injecting a pharmaceutical composition comprising a lipid particle encapsulating the active agent, the lipid particle comprising: from 6 mol % to 10 mol % one or more cationic lipids, from 20 mol % to 65 mol % one or more ionizable lipids; from 35 mol % to 80 mol % one or more neutral lipids; and from greater than 0 mol % to 5 mol % one or more PEGylated lipids. In some cases, the active agent can comprise an anticancer agent, such as an active agent for the treatment of lung cancer.

Also provided are methods of locally administering an active agent to a subject in need thereof. These methods can comprises intramuscularly injecting a pharmaceutical composition comprising a lipid particle encapsulating the active agent, the lipid particle comprising: from greater than 0 mol % to 5 mol % one or more cationic lipids; from 20 mol % to 65 mol % one or more ionizable lipids; from 35 mol % to 80 mol % one or more neutral lipids; and from greater than 0 mol % to 5 mol % one or more PEGylated lipids. In some cases, the active agent can comprise a nucleic acid (e.g., siRNA, mRNA, or any combination thereof). In some cases, the active agent can comprise a vaccine.

Reference will now be made in detail to the present exemplary embodiments, examples of which are illustrated in the accompanying drawings. Other embodiments will be apparent to those skilled in the art from consideration of the specification and practice disclosed herein. The embodiments are further explained in the following examples. These examples do not limit the scope of the claims, but merely serve to clarify certain embodiments. It is intended that the specification and examples be considered as exemplary only, with a true scope and spirit being indicated by the following claims.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1: pH Sensitive Nanoemulsions (PSNEs) for the Delivery of Nucleic Acid Cargos

### Overview

The ability of lipid nanoparticle (LNP) to deliver genetic materials had shown great potential in therapeutics to treat a wide variety of diseases. However, due to the low accessibility of LNP encapsulating genetic payloads to tumor microenvironment, very few of such nanomedicines have been developed and under clinical investigation. QTsome is a LNP platform that uses a combination of quaternary amine-based cationic lipid and a tertiary amine-based ionizable lipid to facilitate drug delivery. Here, we have evaluated and optimized the components of QTsome specifically for delivering nucleic acid cargos. By way of example, we have also developed an antisense oligonucleotide against miR-21 in cancer. QTsome encapsulating AM21 showed superior antitumor activity in multiple tumor models and showed synergistic effects with erlotinib, a tyrosine kinase inhibitor, and atezolizumab, an anti-PDL I therapy.

### Introduction

MicroRNAs (miRNAs) are short non-coding RNAs which can regulate gene expression through mRNA degradation or partial translational repression. miRNA-based gene regulation can take place with miRNA-induced silencing complex (miRISC) complex containing miRNA guide strand complementarily binding with 3′-untranslated region (3′-UTR) on the target messenger RNA (mRNA) by the seeding region of the miRNA, the first 2-7 nucleotides from the 5′end. This canonical interaction usually results in mRNA deadenylation, translational suppression, and mRNA degradation. However, in most cases, the interactions between miRNAs their target mRNA are not entirely complementary. This leads to the idea that a single miRNA would potentially target multiple downstream mRNAs or multiple sites on the same mRNA, suggesting that a broader biological process may be involved in miRNA-based gene regulation. With the robust regulatory effects in biological process and gene expression by miRNA, research also showed that miRNA can not only be found inside the cell, but also migrate to extracellular compartments and systemic fluids, making miRNAs as efficient biomarkers in multiple diseases. The first miRNA used as a biomarker is miRNA-21 (miR-21), where researchers found that high expression of miR-15, miR-21, and miR-210 in the serum of patients with diffuse large B-cell lymphoma (DLBCL) in 2008. Specifically, miR-21 exhibited the largest differential expression in clinical samples and is highly related to disease progression and patient survival. Later, miR-21 overexpression has also been demonstrated a promising biomarker in poor diagnosis and prognosis of many solid tumor types including non-small cell lung cancer (NSCLC) and colorectal cancer. Recently, researcher found that miR-21 may function not only as a biomarker but also as an oncogenic miRNA that regulates epigenetic level of cell apoptosis, DNA repair, cell proliferation, tumor metastasis, and drug resistance by downregulating tumor suppressor genes such as AKT1, DDAH1, PTEN, PDCD4, etc. In addition, research also showed that miR-21 is also associated with pro-tumor immune responses with higher population of M2-macrophages, suggesting that miR-21 may induce drug resistance against not only chemotherapies but also immunotherapies. Regardless of continuous showcase of miRNA as biomarkers in cancer, however, efficient therapeutics targeting pro-tumor miRNAs are underdeveloped.

Antisense oligonucleotide (ASO) is a single-stranded deoxyribonucleotide which is complementary to its target. The mechanism of antisense targeting is to induce gene downregulation by recruiting RNAse H endonuclease activity that could cleaves the heteroduplex formed by ASO and target genes. ASO could not only target mRNA but also oligonucleotides such as miRNA or small-interfering RNA (siRNA), which makes it a promising approach for therapeutically inhibiting miRNA. However, the unmodified anti-miRNA antisense oligonucleotides (ASOs) are sensitive to nucleases which are easily cleared from systemic fluids. To overcome this barrier, many types of chemical modifications of ASO have been introduced. Modification of the phosphate backbone, the nucleic acid base, and the ribose sugar moiety has been extensively employed to improve drug pharmacokinetics, pharmacodynamics and biodistribution. Chemical conjugation of ASO, including small molecules, peptides, aptamers, antibodies, has also been developed to improve tissue-specific biodistribution and therapeutic efficacy. Nonetheless, free drug of ASO contains high density of negative charges which make them less accessible to tumor microenvironment (TME) through cellular uptake. Therefore, an efficient delivery platform is needed for therapeutic ASO.

A QTsome is a lipid nanoparticle (LNP) platform that utilizes a combination of a quaternary amine-based cationic lipid and a tertiary amine-based ionizable lipid to facilitate drug delivery. The design of using a cocktail of cationic lipid and ionizable lipid in QTsome can achieve an improved pH-dependent drug loading and releasement profile. Preliminary studies have shown that traditional design of QTsome was able to deliver oligonucleotides. However, traditional QTsomes (QTsome Original) contains high amount of PEG-lipids and outdated functional lipids which impedes cellular uptake and releasement of nucleic acid cargos into cytoplasm, making the gene delivery by QTsome Original less efficient than other emerging LNP platforms from Moderna, Pfizer/BioNTech, Alnylam Pharmaceuticals, etc. In this example, QTsome Original was optimized into QTsome Plus (QTPlus) by lowering the amount of cationic lipid and PEG lipid, increasing the amount of ionizable lipid, and screening the candidates for ionizable lipid and helper lipid. Eventually, QTPlus exhibited much enhanced gene delivery, including oligonucleotides and mRNA, compared with QTsome Original and LNP standard with Moderna formulation. QTPlus was further utilized to deliver a 16-mer anti-miR-21 ASO (AM21) and showed significant miR-21 inhibition through downstream gene regulation. In addition, QTPlus-encapsulating AM21 (QTPlus-AM21) showed significant antitumor activity in A549 NSCLC and MC38 colorectal tumor models. QTPlus-AM21 significantly induced chemo sensitization in NSCLC in the presence of erlotinib, a tyrosine kinase inhibitor (TKI), and showed enhanced antitumor immunity in combination with atezolizumab, an anti-PDL I therapy, suggesting that QTPlus-AM21 may also be a strong antitumor candidate for the combination therapies with chemotherapies and immunotherapies.

### Materials and Methods

Materials. 1,2-dioleoyl-3-trimethylammonium-propane (DOTAP) was purchased from MedChemExpress (Monmouth Junction, NJ, United States). 1-(2,3-bis(((9Z,12Z)-octadeca-9,12-dien-1-yl)oxy)propyl)pyrrolidine (A-066) was purchased from Hangzhou Dragonpharm Co., Ltd (Hangzhou, China). Cholesterol was purchased from Avanti Polar Lipids, Inc (Birminghan. AL, United States). 1,2-dioleoyl-sn-glycero-3-phosphoethanolamine (DOPE) and 1,2-Dimyristoyl-rac-glycero-3-methoxypolyethylene glycol-2000 (DMG-PEG2000) were purchased from NOF America (Cambridge, MA, United States). CleanCap®Firefly Luciferase (FLuc) mRNA was purchased from TriLink Biotechnologies (San Diego, CA, United States). AM21 were purchased from Integrated DNA Technologies, Inc. (Coralville, IA, United States) (20-mer AM21 sequence: 5′-+C*+A*+A*+C*A*+T*C*+A*G*T*+C*+T*G*A*+T*A*+A*G*+C*+T-3′. 16-mer AM21 sequence: 5′-A*+T*+C*A*G*+T*+C*+T*G*A*+T*A*A*G*+C*+T-3′. Scramble 16-mer oligonucleotide sequence: 5′-+C*A*C*G*+T*+C*+T*A*+T*A*+C*G*+C*+C*+C*A*-3′. “+” represents locked nucleic acid, LNA, bases. “*” represents phosphorothioated backbone). Erlotinib was purchased from Cayman Chemical (Ann Arbor, MI, United States). Atezolizumab was kindly provided by the Arthur G. James Cancer Hospital from The Ohio State University (Columbus, OH, United States).

QTPlus-mRNA and QPlus-AM21 Formulation and Characterization. For QTPlus-mRNA specifically, DOTAP, A-066, DOPE, cholesterol, and DMG-PEG2000 were prepared at a molar ratio of 1.5/50/12/35/1.5 in ethanol. FLuc mRNA were prepared in DEPC-treated water. The lipid mixture in ethanol were directly injected into FLuc mRNA phase where QTPlus self-assembly and mRNA encapsulation take place simultaneously. This QTPlus formulation protocol was referred as 1-Step formulation.

Empty QTsome Original and QTPlus were prepared by hand-rapid injection of the lipid mixture into acetic acid buffer. DOTAP, A-066, DOPE, cholesterol, and DMG-PEG2000 were prepared at a molar ratio of 1.5/50/12/35/1.5 in ethanol. FLuc mRNA, AM21 and scramble oligonucleotide solutions were prepared in DEPC-treated water. Oligonucleotide solutions were mixed with empty QTsome Original or QTPlus phase with equal volume to reach the final lipid-to-oligo ratio at 10/1 (w/w). This formulation protocol was referred as 2-Step formulation. The final lipid concentration of QTsome was 10 mg/ml, and the final oligonucleotide concentration was 1 mg/ml. Particle sizes of QTsome-encapsulating oligonucleotides were measured by dynamic light scattering (DLS) using a NICOMP NANO ZLS Z3000 (Entegris, Billerica, MA, United States). Gel electrophoresis was performed using 1% agarose gel loaded with 1 ug oligonucleotide per well. Fluorescence imaging of ethidium bromide was taken after 20 minutes gel electrophoresis at 100 volts. Cryo-EM images of QTPlus-AM21 was obtained from Center for Electron Microscopy and Analysis at the Ohio State University (Columbus, OH, United States).

Cell Culture. RAW 264.7 murine macrophage cell line and MC38 murine colorectal carcinoma cell line were kind gifts given by Dr. Peixuan Guo and Dr. Christopher Coss at The Ohio State University College of Pharmacy, respectively. THP-1 human monocyte cell line was kindly provided by Dr. Joshua Englert at The Ohio State University Wexner Medical Center. KB and A549 cell lines were purchased from Millipore Sigma (Burlington, MA, United States). RAW 264.7 and MC38 were grown in DMEM supplemented with 10% FBS and 1× antibiotic-antimycotic. THP-1, KB, and A549 were grown in RPMI supplemented with 10% FBS. Cells were maintained at 37° C. and grown under a humidified atmosphere containing 5% CO₂.

In Vitro Bioluminescent Assay. HEK293, A549, and KB were seeded at 3000 cells/well in 96-well plates 24 hours prior to treatments. Cells were treated with 0.1 ug of FLuc mRNA in QTsome Original or QTPlus. Cells were allowed to uptake QTsome Original or QTPlus encapsulating mRNA overnight. The mRNA expression was determined by Bright-Glo™ Luciferase Assay System (Promega, Madison, WI).

In Vitro Gene Regulation. KB, A549, THP-1, MC38, and RAW264.7 cells were seeded at 3×10⁵ cells/well in 6-well plates 24 hours prior to treatments. Cells were treated with scramble oligonucleotide or AM21 in free solution, lipofectamine, QTsome Original, and QTPlus. Total RNA was extracted using TRI reagent (Zymo Research) per manufacturer protocol. cDNA was prepared by high-capacity cDNA reverse transcription kit (Invitrogen, Waltham, MA, USA), and real-time qPCR (RT-qPCR) was done using SsoAdvanced™ Universal SYBR® Green Supermix (Bio-Rad Laboratories, Hercules, CA) on a QuantStudio 7 Flex Real-time PCR System. All the RT-qPCR primers were purchased from Sigma-Aldrich. Beta-actin (Actb) was selected as the housekeeping gene control. The relative amount of RNA level was calculated and compared according to the 2-ΔΔCt method.

Colony Formation Assay. A549 cells were seeded at 100 cells/well in 24-well plates 24 hours prior to treatments. Cells were treated with 16-mer and 20-mer AM21 in free solution and QTPlus at concentrations of 1 uM and 10 uM. Cells were allowed to form colony for up to 1-week followed with fixation by methanol and colony detection by crystal violet.

MTS Assay. A549 cells were seeded at 3000 cells/well in 96-well plates 25 hours before treatments. Cells were treated with QTPlus AM21 (concentrations from 0 nM to 6.4 uM), erlotinib (0 uM to 200 uM), and a combination of QTPlus AM21 and erlotinib at a fixed concentration ratio (QTPlus AM21/erlotinib of 6.4/200). After 72-hour treatment, cell viability was examined by CellTiter 96® AQueous One Solution (Promega, Madison, WI) per manufacturer protocol. The synergistic effects by QTPlus-AM21 and erlotinib was determined by CompuSyn software (The ComboSyn, Inc.).

Macrophage-Tumor Cell Co-culture Study Wound Healing Assay. MC38 and RAW264.7 cells were seeded on 6-well plates in a total number of 6×10⁶ cells per well with a fixed macrophage-to-tumor cell ratio of 3/1. Cells were treated with 400 uM of AM21 in free solution or QTPlus with or without 1 ug/ml of lipopolysaccharide (LPS) stimulation. Cells were incubated for 24 hours at 37° C. and collected for flow cytometry analysis.

For wound healing study, a scratch wound healing model was conducted to examine the migratory ability of MC38 cells in the presence of macrophages following treatment. A scratch wound across the well was made using a 10 ul pipet tip immediately before treatment. Cells were washed by PBS and incubated with complete media containing 400 uM of AM21 in free solution and QTPlus with or without 1 ug/ml of LPS stimulation. Cells were allowed to proliferate at 37° C. for 24 hours. Distances between edges of the wound were measured by Nikon Eclipse Ti-S microscope (Nikon, Tokyo, Japan).

Enzyme-linked Immunosorbent Assay (ELISA). Human PTEN Matched Antibody Pair Kit was purchased from Abcam (Cambridge, UK). Human EGFR Matched ELISA Antibody Pair Set was purchased from Sino Biological (Beijing, China). A549 cells were seeded at 8×10⁵ cells/plate in 60 mm culture dishes 24 hours prior to treatment. Cells were treated with 10 ug of QTPlus-AM21 and 20 uM of erlotinib individually or in combination. After overnight treatment, cells were harvested and homogenized in Pierce RIPA buffer (Thermo Fisher Scientific). Total proteins were extracted after incubating on ice for 30 minutes and centrifuged at 14000×g for 30 minutes at 4° C. Protein concentrations were quantified and unified by Pierce BCA Protein Assay Kit (Themo Fisher Scientific). PTEN and EGFR concentrations were measured per manufacturer's protocol.

In Vivo Antitumor Efficacy Study. To determine the antitumor efficacy of QTsome Original-encapsulating AM21, A549 NSCLC xenograft mouse model was generated by subcutaneously inoculating nude mice with 1×10⁷ cells per mouse on the right flack. Treatments were initiated once tumors reached approximately 100 mm³. Mice (n=5) were intravenously treated with saline, 4 mg/kg QTsome Original-encapsulating ASO against AKT, and 3.5 mg/kg QTsome Original-encapsulating AM21. The study was conducted by Bioduro-Sundia (San Diego, CA, United States).

To evaluate the antitumor efficacy of QTPlus-AM21 and the combination therapy of QTPlus-AM21 and erlotinib, A549 xenograft mouse model was generated by subcutaneously inoculating nude mice with 2.5×10⁶ cells per mouse on the right flack. Treatments were initiated once tumors reached 50-100 mm³. Mice (n=3) were intravenously treated with saline, 3 mg/kg QTPlus-encapsulating scramble oligonucleotide, 3 mg/kg QTPlus-AM21, orally treated with 50 mg/kg erlotinib, or QTPlus-AM21/erlotinib combination (3 mg/kg QTPlus AM21 and 50 mg/kg erlotinib).

To evaluate the combination therapy of QTPlus-AM21 and anti-PDL I therapy, MC38 murine colorectal syngeneic model was generated by subcutaneously inoculating C57BL/6 mice (obtained from Charles River Laboratories) with 1×10⁶ cells per mouse on the right flack. Treatments were initiated once tumors reached 50-100 mm³. Mice (n=5) were intraperitoneally treated with saline, 3 mg/kg QTPlus-encapsulating scramble oligonucleotide, 3 mg/kg QTPlus-AM21, 10 mg/kg atezolizumab, and QTPlus-AM21/atezolizumab combination (3 mg/kg QTPlus-AM21 and 10 mg/kg atezolizumab).

All mice were dosed every 3 days for 5 doses. Tumor growth and body weight were monitored, and the tumor volumes were calculated according to the formula:

$$Tumor Volume = \frac{Length \times {Width}^{2}}{2}$$

All animal studies were reviewed and approved by The Ohio State University Institutional Laboratory Animal Care and Use Committee (IACUC). All mice were euthanized one day after the last dose to peak the immune activation and protein expression in TME. Terminal Tumor growth inhibition (% TGI) was determined by the formula:

$$\% T G I = \frac{1 - ( {T}_{t} / {T}_{0} ) / ( {C}_{t} / {C}_{0} )}{1 - {C}_{0} / {C}_{t}} \times 100 \%$$

where Tₜ stands for average tumor volume of treatment group at the day of measurement, T₀ stands for average tumor volume of treatment group at day 0, Cₜ stands for average tumor volume of the control group at the day of measurement, and C₀ stands for average tumor volume of the control group at day 0. % TGI>50% was considered meaningful.

Flow Cytometry. Alexa Fluor 700 anti-mouse CD45 (30-F11), APC/Cyanine7 anti-mouse CD3e (145-2C11), FITC anti-mouse CD4 (RM4-5), PE/Cyanine7 anti-mouse CDSa (53-6.7), PE anti-mouse FOXP3 (MF-14), and BV-650 anti-mouse NK1.1 (PK136) were used for detection of lymphoid cell populations in mouse tumor. FITC anti-mouse F4/80 (BM8), PE anti-mouse CD206 (C068C2), BV-605 anti-mouse CD86 (PO3), APC-Cy7 anti-mouse CD11b (M1/70), and BV-650 anti-mouse Gr-1 (RB6-8C5) were used for detection of myeloid cel populations in mouse tumor. All the fluorophore-conjugated antibodies and True-Nuclear Transcription Factor Buffer Set for FOXP3 staining were purchased from BioLegend (San Diego, CA, USA). Single-cell suspensions of tumor tissues in FACS staining buffer were stained per manufacturer protocol. Stained cells were analyzed using an LSR II flow cytometer in Flow Cytometry Shared Resources (FCSR) at The Ohio State University Comprehensive Cancer Center.

Statistical Analysis. All studies were done in triplicate. Data are presented as means±standard deviations unless otherwise indicated. Statistical analysis will be conducted using Microsoft Excel. One-way ANOVA was used to determine variances in means between two or more treatment groups. Student's t test was used as a post-hoc analysis to determine statistically significant differences between any two groups. A p-value of 0.05 was selected as the cutoff for statistical significance.

### Results

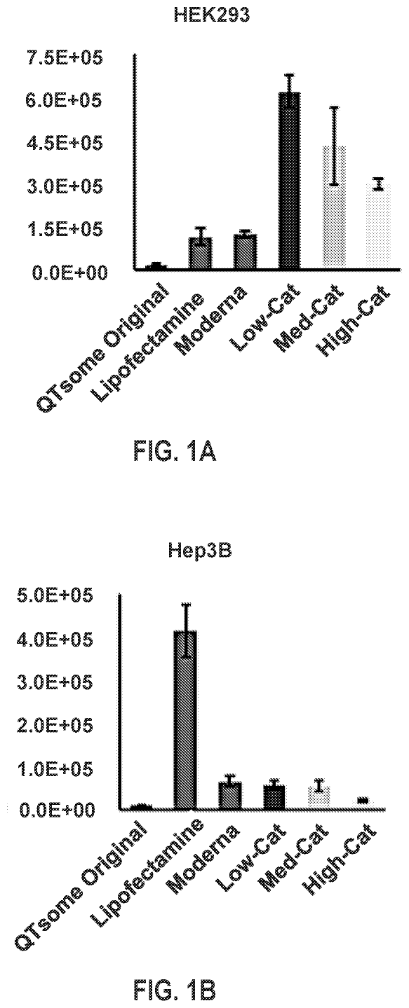

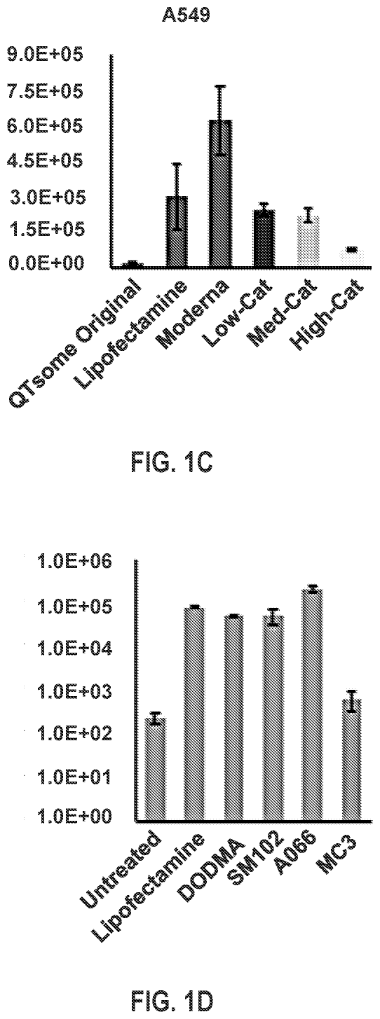

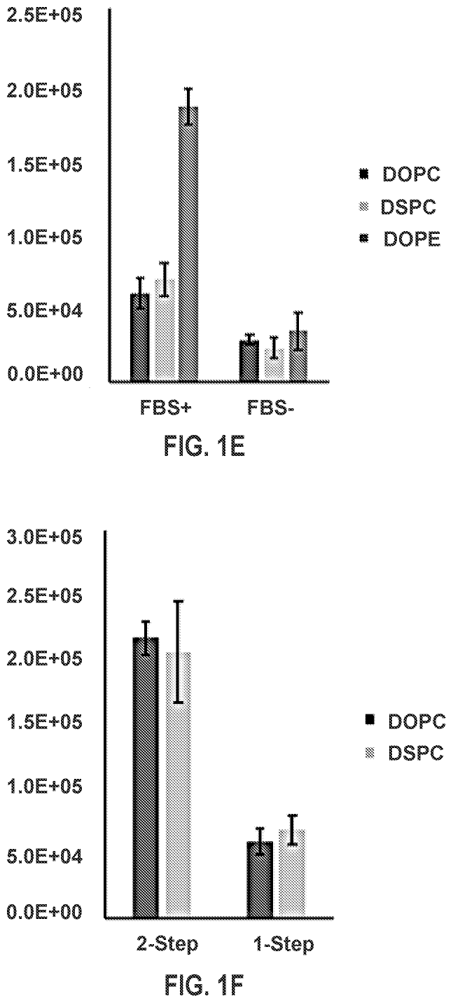

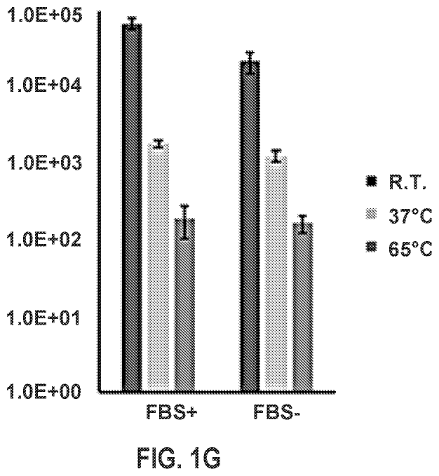

Optimizing QTsome Original into QTPlus Formulation. The mRNA delivery efficiency of QTPlus with different components were compared with QTsome original, lipofectamine, and LNP standard from Moderna (Moderna STD) in vitro (FIGS. 1A-1G). In terms of cationic lipid, higher amount of cationic lipids exhibited higher mRNA delivery efficiency (FIGS. 1A, 1B, and 1C). The mRNA delivery efficiency by QTPlus with different types of commercialized ionizable lipids were also evaluated. In this analysis, A-066 was the most suitable for mRNA delivery in QTsome platform (FIG. 1D), with DOPE the most suitable as helper lipids (FIG. 1E). QTPlus were also formulated based on two different protocols (1-Step and 2-Step). It was surprising that, in the contrary to standard 1-Step LNP/mRNA formulation protocol, the QTPlus-mRNA developed by 2-Step formulation exhibited superior mRNA delivery to HEK293 cells (FIG. 1F). Lastly, the mRNA expression in vitro was impaired as the formulation temperature increased (FIG. 1G). Therefore, QTPlus-mRNA were developed under room temperature instead of 60° C. pre-incubation prior to formulation.

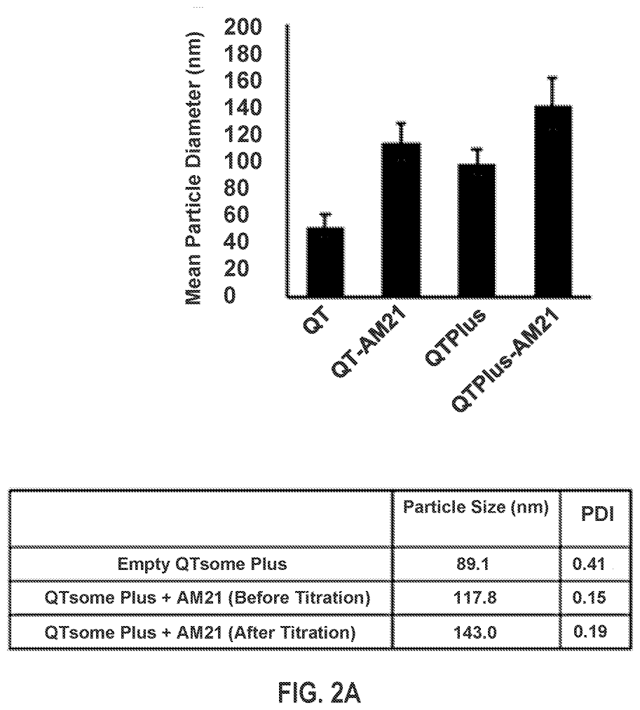

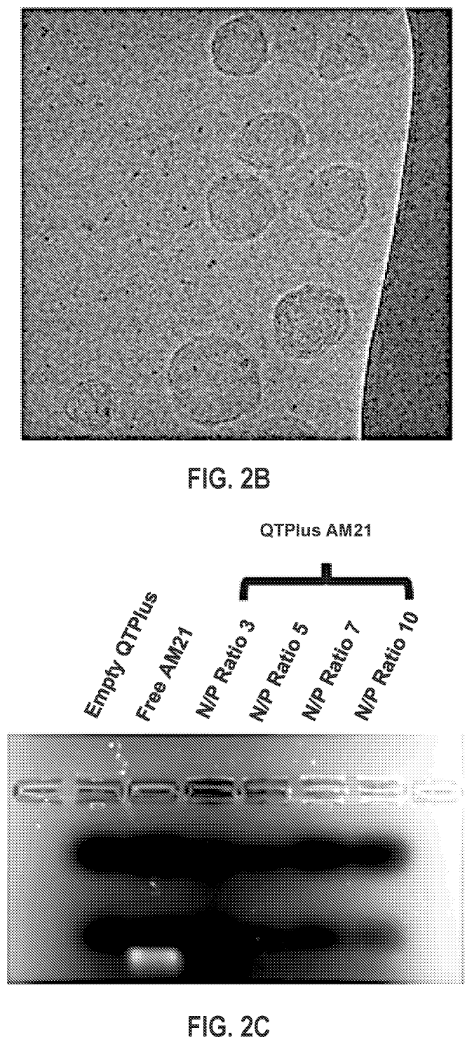

QTPlus Delivering Both mRNA and Oligonucleotides. Particle sizes of the empty QTPlus and the QTPlus-AM21 were larger than the empty QTsome Original and the QTsome Original-AM21 which are 80-90 nm (empty QTPlus) and 110-120 nm (QTPlus-AM21) (FIG. 2A). The particle sizes of QTPlus-AM21 also slightly increased to 140 nm after titrating the final product to pH-neutral solution (FIG. 2A). The QTPlus-AM21 exhibited a compact sphere structure with a single lipid layer wrapped outside (FIG. 2B) and was capable to encapsulate any amount of oligonucleotide of N/P ratio from 3 to 10 (FIG. 2C).

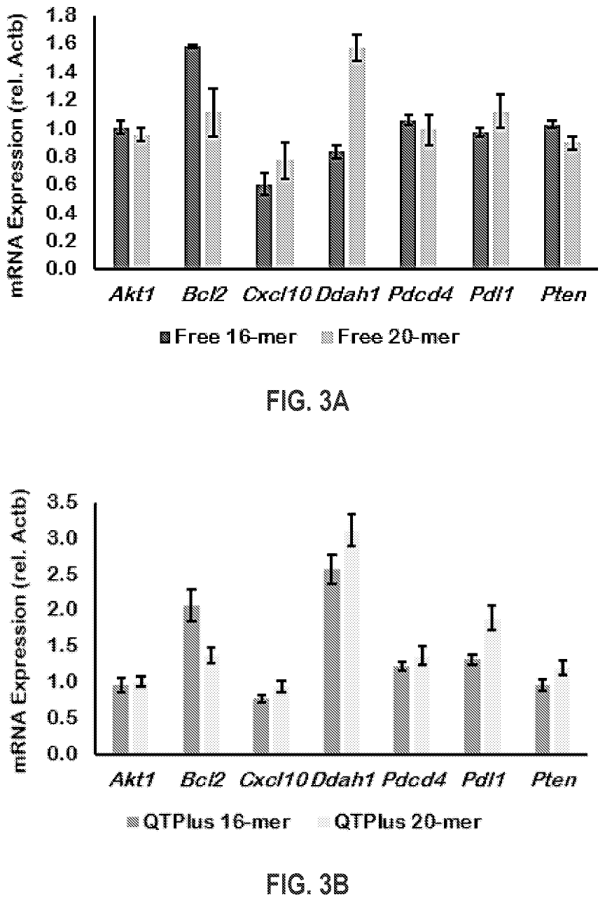

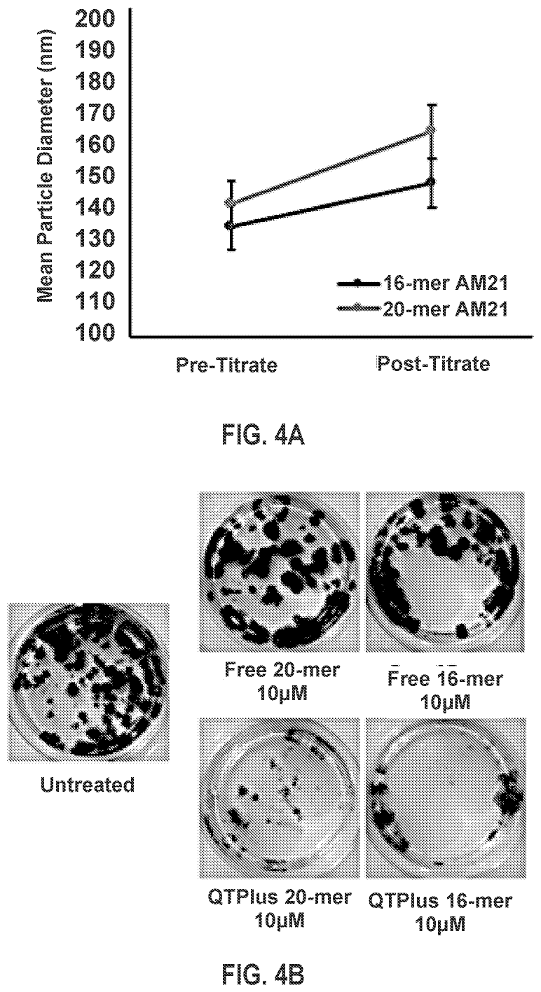

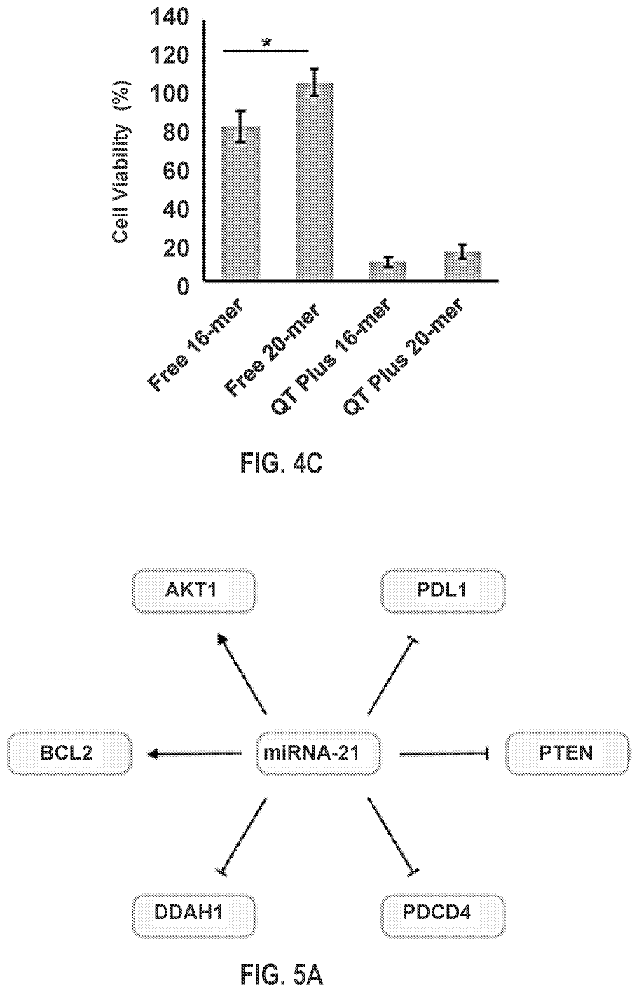

16-mer Versus 20-mer AM21. Different length AM21 oligonucleotides were designed to evaluate the sequence length-dependent gene regulation and antitumor effects (FIGS. 3A-3B and FIGS. 4A-4C). The 16-mer and 20-mer AM21 exhibited different miR-21 downstream gene regulation profiles when treated into A549 cells in free solution and QTPlus formulation (FIGS. 3A-3B). However, 16-mer AM21 was superior in reducing the particle aggregation when titrating the final QTPlus-AM21 product to pH-neutral solution (FIG. 4A). In addition, 16-mer AM21 was superior in inhibiting A549 cell growth in vitro when treated with free solution or QTPlus formulation (FIGS. 4B and 4C).

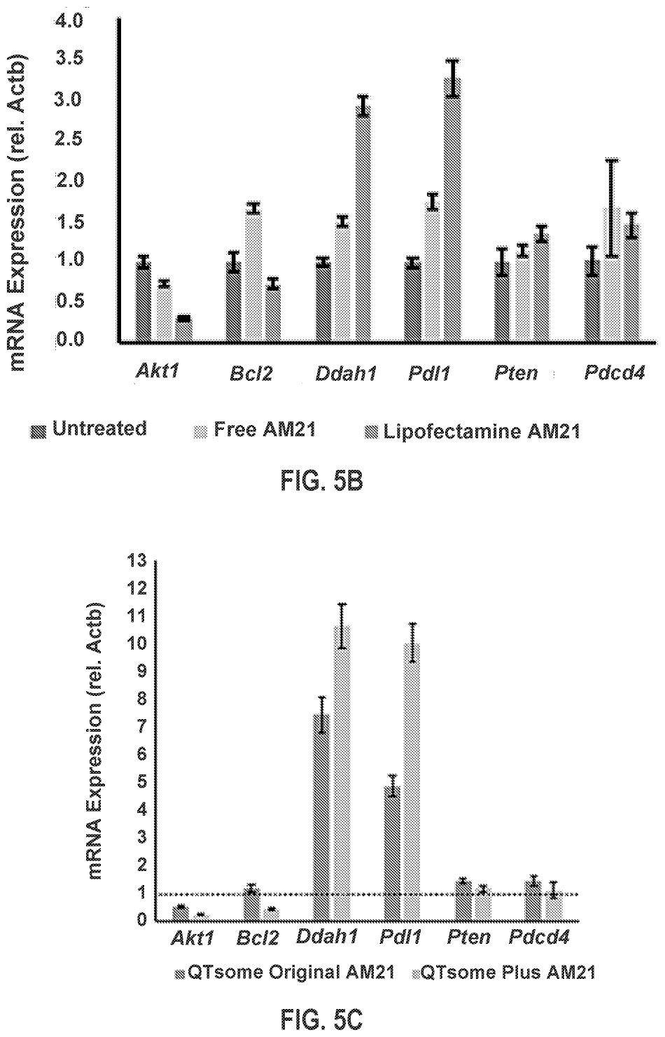

Gene Regulation by QTPlus-AM21 in Tumor Cells In Vitro. In KB and A549 cells, AM21 consistently induced Akt1 downregulation, Ddah1 upregulation, and Pdl1 upregulation (FIGS. 5B and 5C). However, the roles of AM21 in regulating Bcl2, Pten, and Pdcd4 expression are controversial when AM21 is transfected by free solution and QTsome Original or QTPlus to cells (FIGS. 5B and 5C). Based on these profiles, QTPlus-AM21 showed the highest gene regulation levels in A549 cells compared with KB and Hep3b cells in vitro (Table 1). Again, QTPlus was also demonstrated to exhibit higher gene delivery efficiency than QTsome Original (FIG. 5C).

TABLE 1
miR-21 downstream gene regulation by QTPlus-
AM21 in KB, AS49, and Hep3b cell lines.

| miR21 Downstream | KB | A549 | Hep3b |
|---|---|---|---|
| Genes of Interest | mRNA Expression ± STD | | |
| Akt1 | 0.89 ± 0.10 | 0.22 ± 0.02 | 1.67 ± 0.30 |
| Bcl2 | 1.20 ± 0.11 | 0.44 ± 0.05 | 0.89 ± 0.23 |
| Ddah1 | 1.27 ± 0.12 | 10.64 ± 0.80 | 3.68 ± 0.94 |
| Pdl1 | 0.99 ± 0.12 | 10.03 ± 0.69 | 4.07 ± 1.94 |
| Pten | 1.08 ± 0.10 | 1.16 ± 0.11 | 0.71 ± 0.14 |
| Pdcd4 | 1.25 ± 0.15 | 1.10 ± 0.28 | 0.68 ± 0.18 |

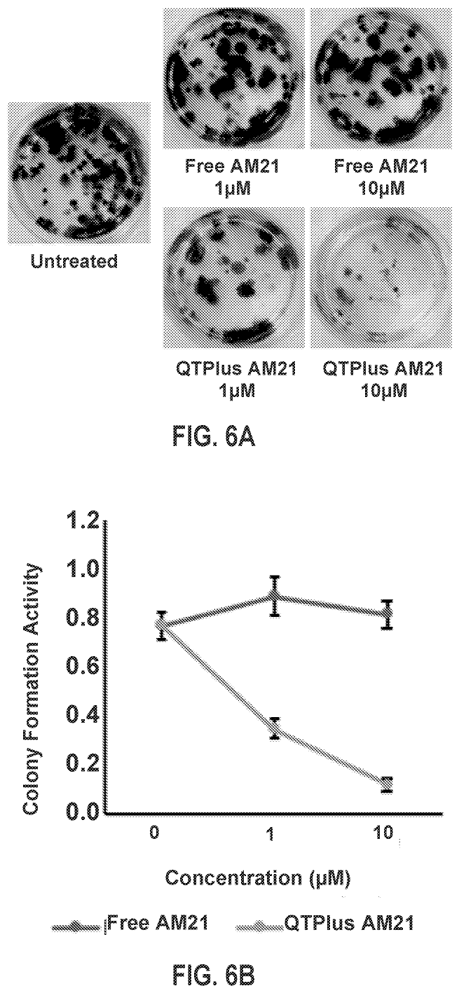

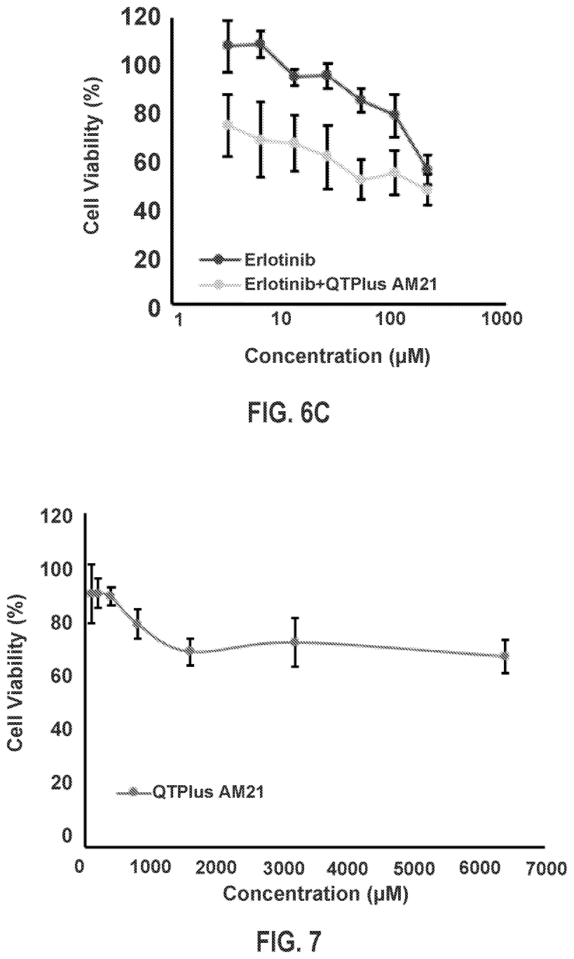

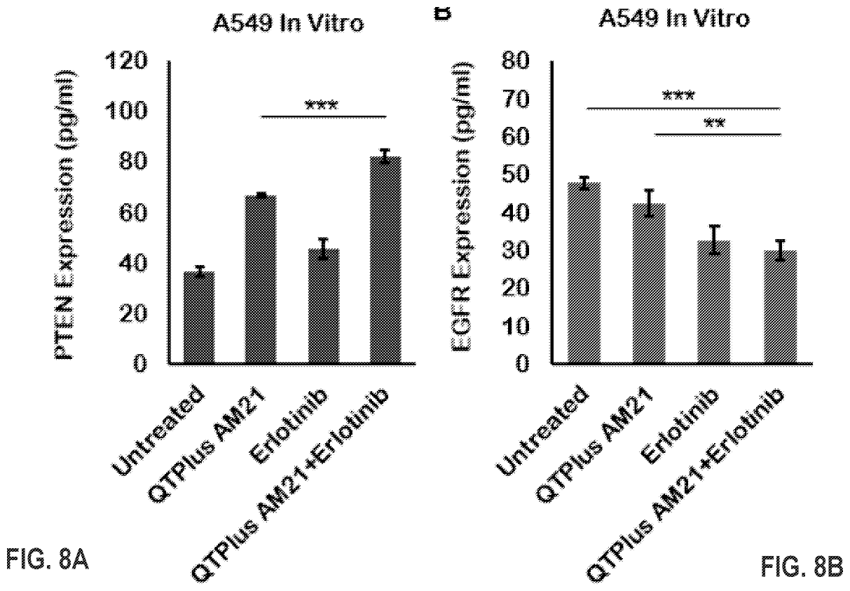

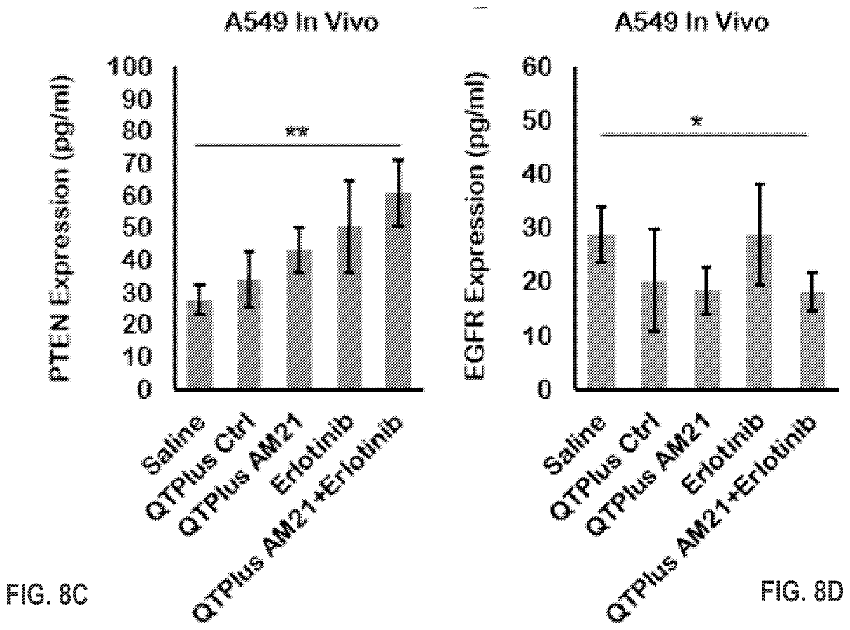

Synergistic Effect between QTPlus-AM21 and Erlotinib. QTPlus-AM21 did not exhibit acute cytotoxicity in A549 cells when treated in a ranged concentration up to 6.4 uM (FIG. 7). However, when AM21 were long-termly treated in A549 cells to evaluate colony formation, QTPlus-AM21 exhibited much higher inhibitory effect in colony formation compared with free AM21 (FIGS. 6A and 6B). Interestingly, QTPlus-AM21 could sensitize A549 cells to the erlotinib cytotoxicity when QTPlus-AM21 were treated together with erlotinib at a fixed concentration ratio (FIG. 6C). The combination index between QTPlus-AM21 and erlotinib was 0.60, and the dose reduction index (DRI) for QTPlus-AM21 and erlotinib were 6.26 and 2.26 respectively. Combination treatment of QTPlus-AM21 and erlotinib also enhanced PTEN expression (FIG. 8A) and inhibited EGFR expression (FIG. 8B) pharmacologically in vitro based on ELISA results.

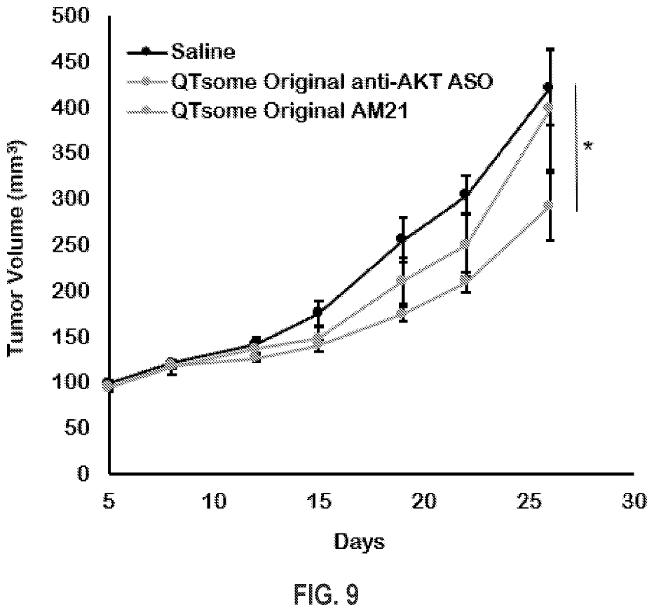

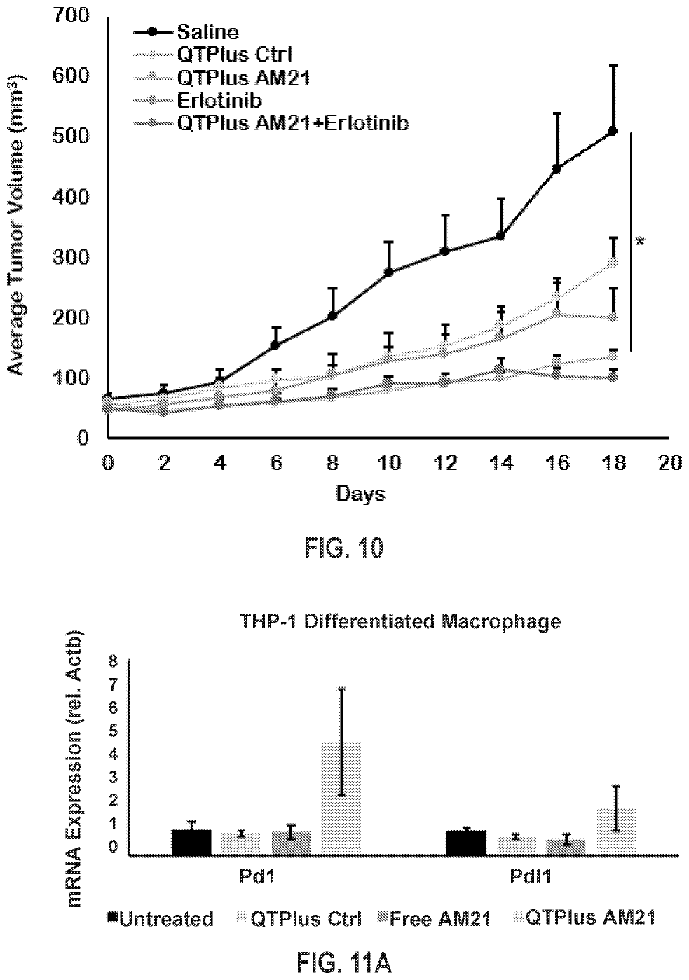

In Vivo Antitumor Activity of QTsome/QTPlus-AM21 against NSCLC. QTsome Original was initially used for AM21 delivery in vivo. QTsome Original encapsulating ASO against AKT (QTsome Original-anti-AKT ASO) was also designed as a control group to compare with antitumor activity between inhibiting miR-21 and one of its downstream oncogenes. Although the antitumor efficacy of QTsome Original-AM21 was limited, it is still higher than QTsome Original-anti-AKT ASO (FIG. 9). When QTPlus were applied to delivery AM21 in vivo, the antitumor responses were significantly increased (TG1% from 37.9%+30.3 of QTsome Original-AM21 to 81.0%+4.9 of QTPlus-AM21). Though not statistically significant, the combination therapy of QTPlus-AM21 and erlotinib showed higher antitumor activity in vivo (with TGI % of 84.4%+7.2) (FIG. 10). In addition, synergistic PTEN upregulation and EGFR downregulation was observed in vivo by the combination therapy of QTPlus-AM21 and erlotinib (FIGS. 8C and 8D) No significant differences in body weight in mice treated with QTPlus-AM21 and erlotinib individually or in combination had minor systemic toxicity.

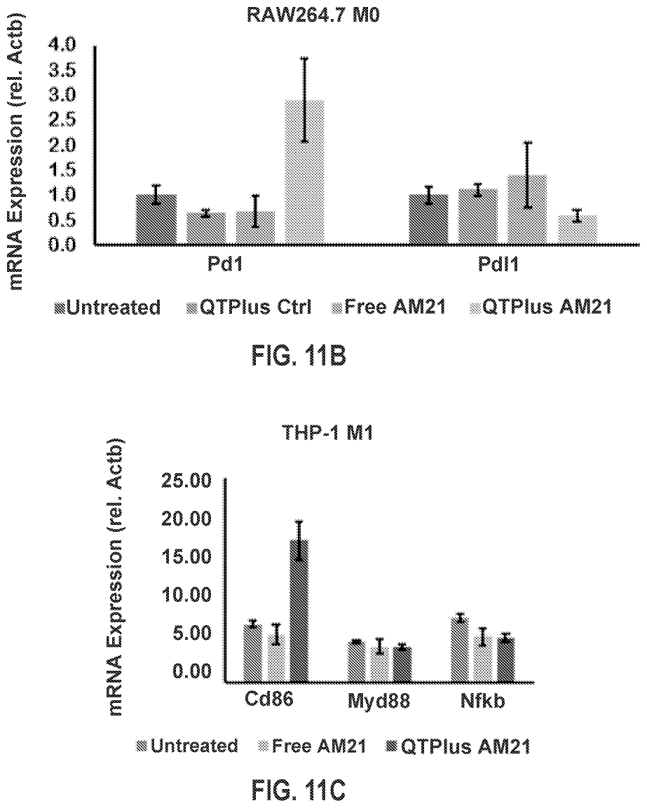

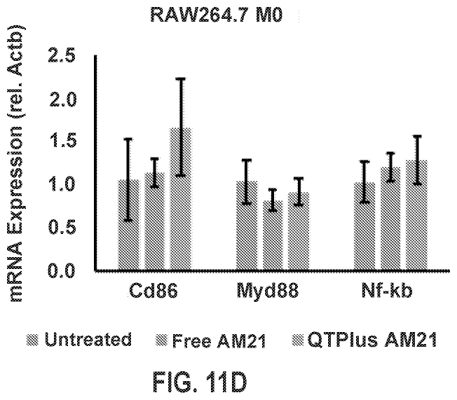

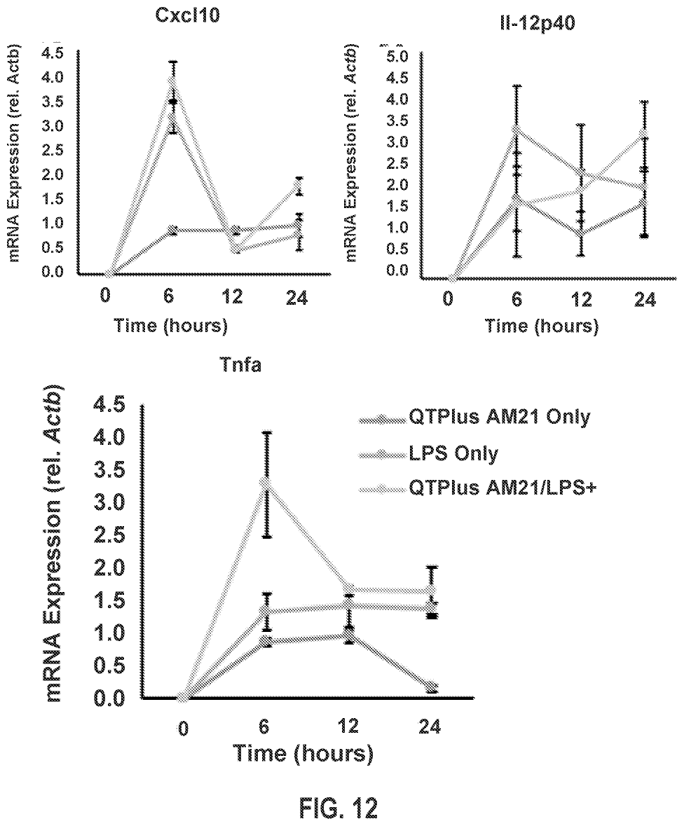

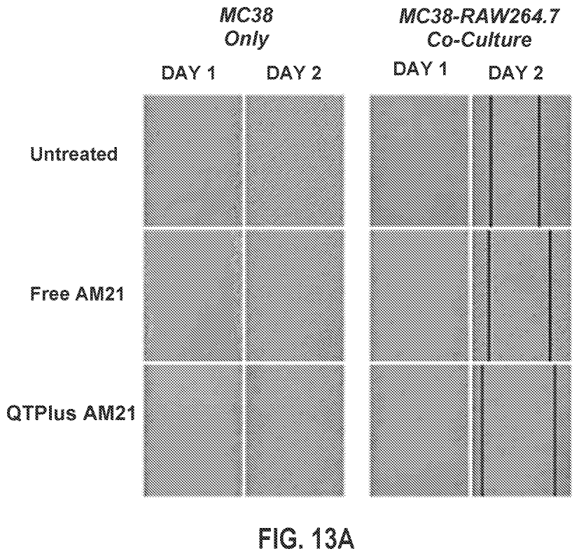

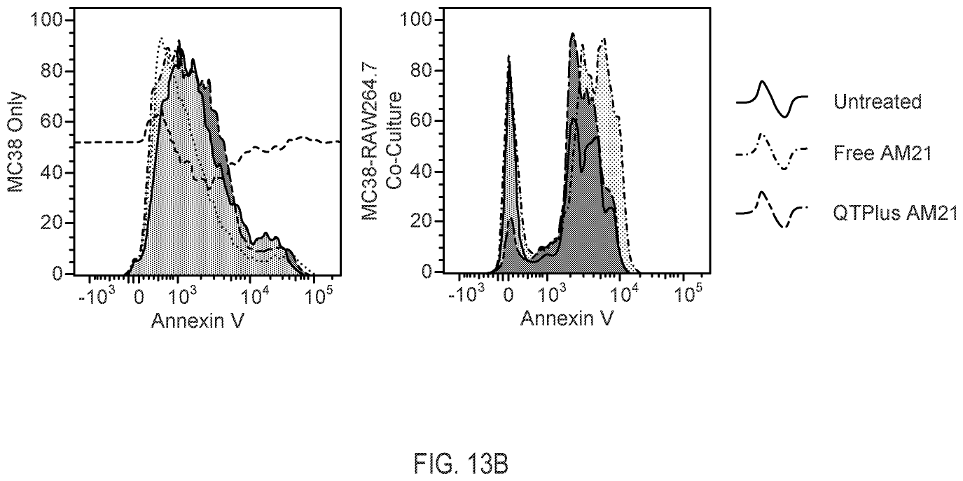

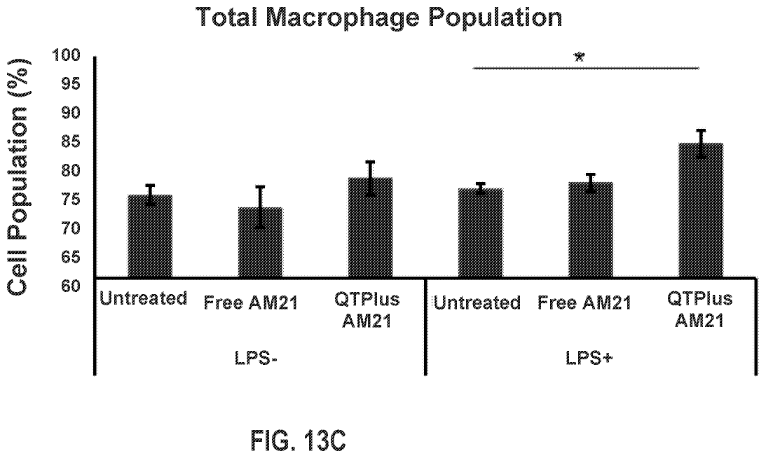

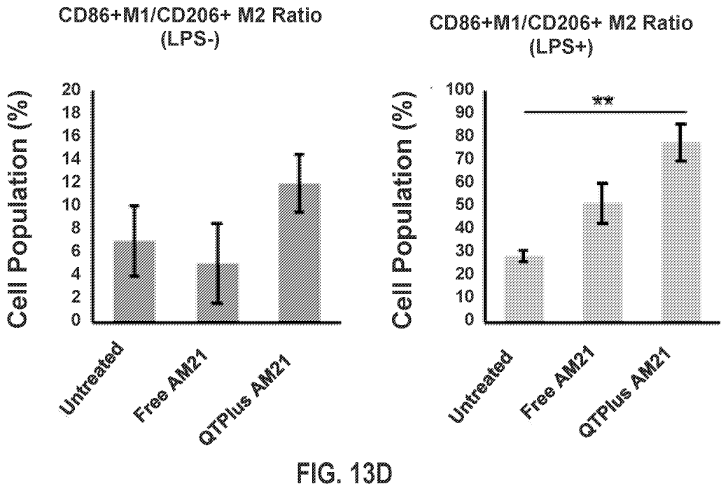

Immunoregulation by QTPlus-AM21. Pdl upregulation was observed in naïve human and murine macrophages without LPS stimulation (FIGS. 11A and 1B). In addition, QTPlus-AM21 could enhance Cd86 upregulation in both human and murine macrophage cell lines when they are polarized into M1 population by LPS stimulation (FIGS. 11C and 11D). The activated M1 macrophages treated with QTPlus-AM21 also enhanced Cxcl10, Il-12p40, and Tnfa expression in vitro (FIG. 12). When mouse macrophages RAW264.7 were co-cultured with mouse colorectal cancer cells MC38, treatment with QTPlus-AM21 could increase macrophage proliferation and polarization into M1 population (FIGS. 13C and 13D). This macrophage polarization by QTPlus-AM21 is also associated with increased apoptosis in MC38 cancer cell population (FIG. 13B) which eventually decreased MC38 growth and wound-healing effects in vitro (FIG. 13A).

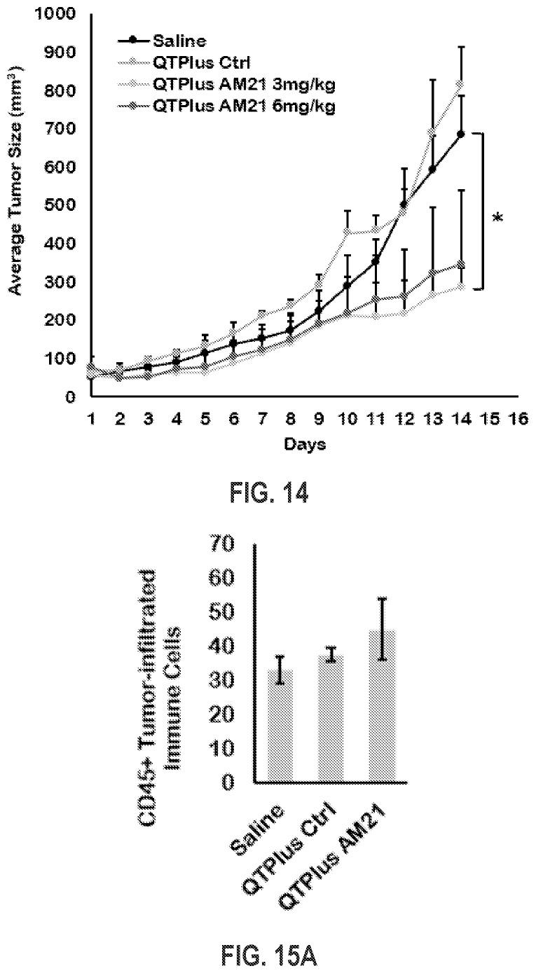

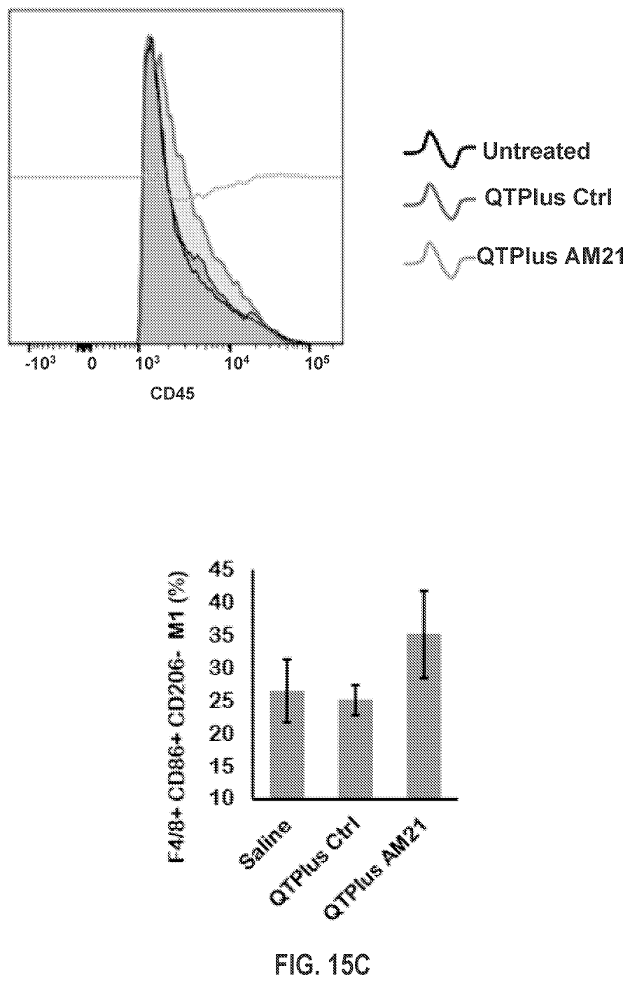

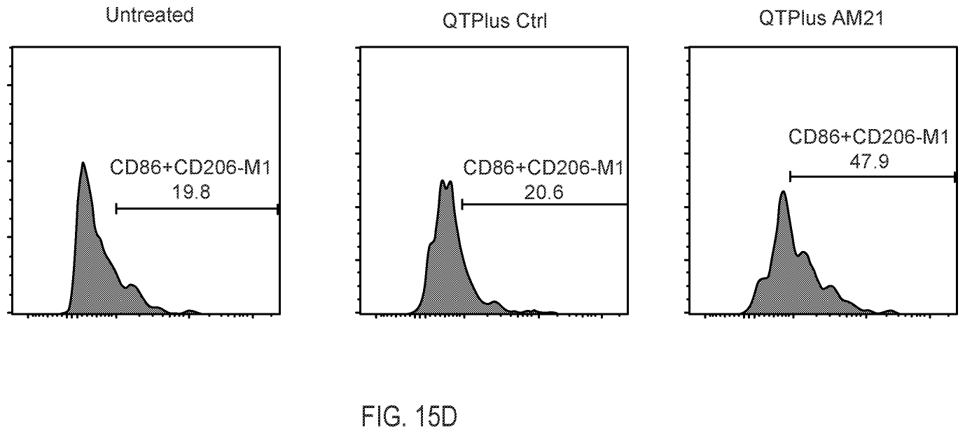

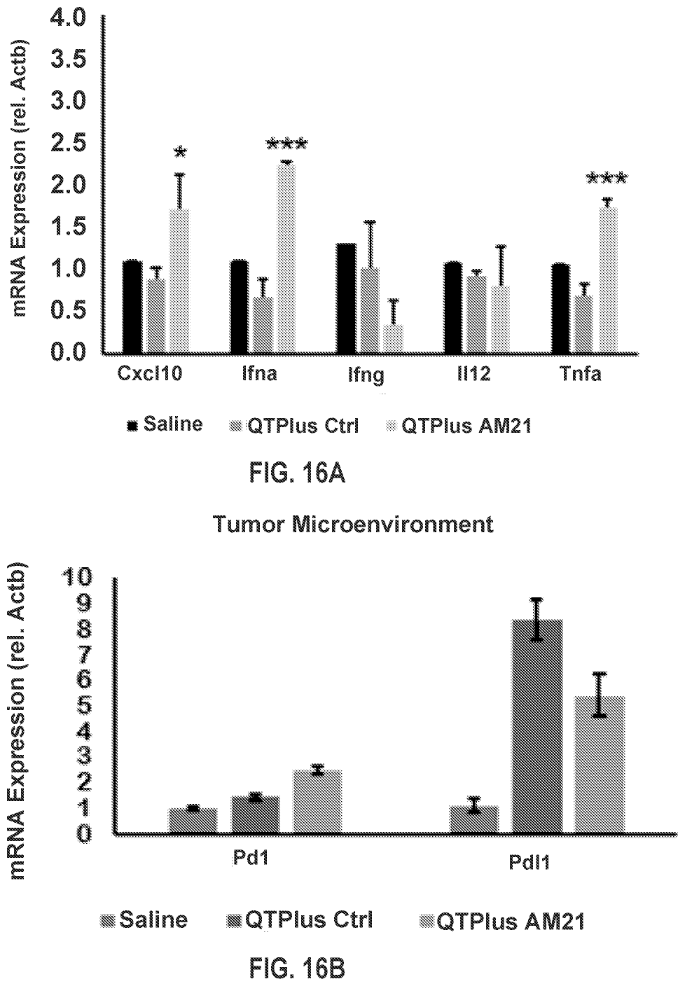

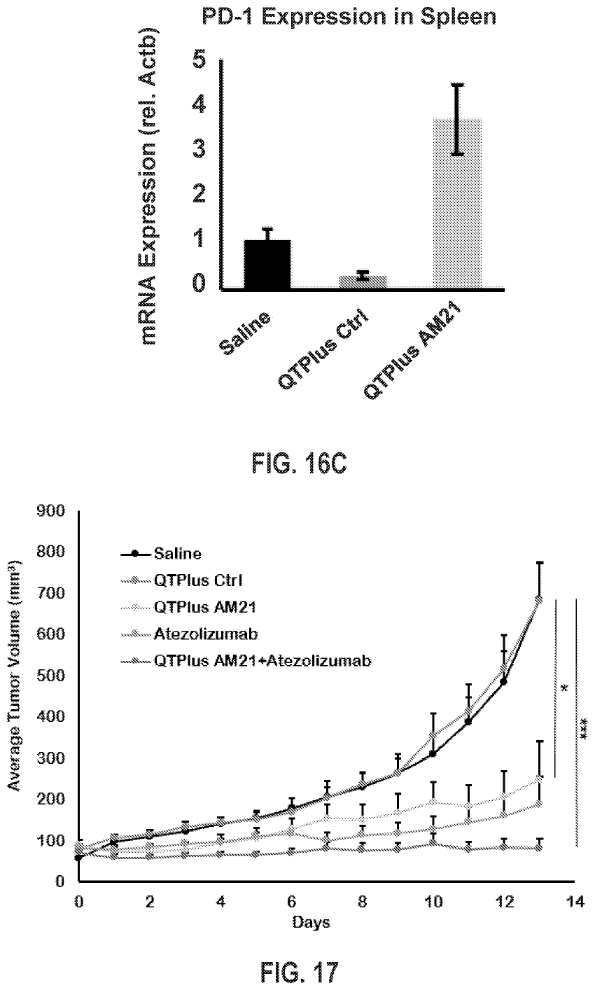

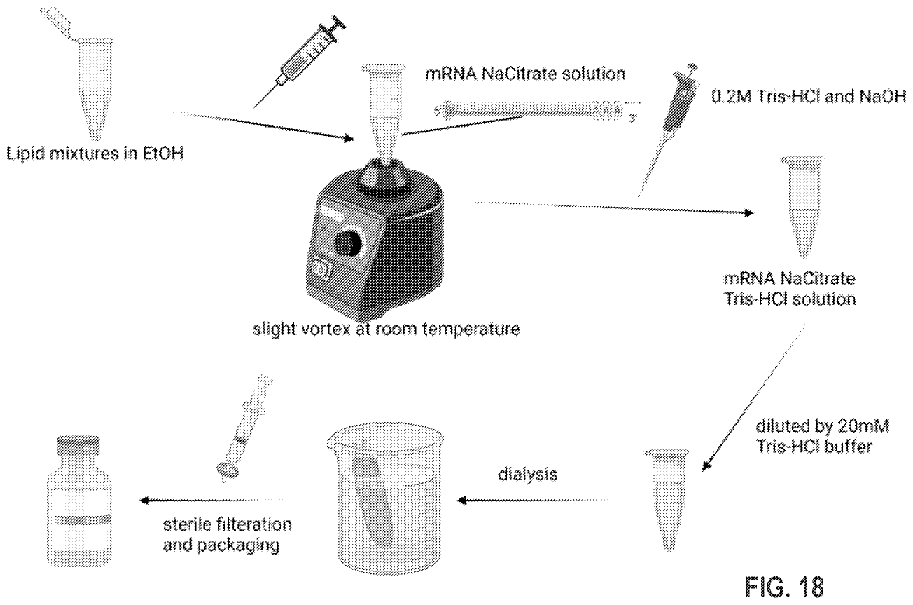

Preliminary animal studies showed that QTPlus-AM21 also exhibited antitumor activity in MC38 syngeneic mouse model (FIG. 14) Mice treated QTPlus-AM21 exhibited final TG1% of 68.33%+12.4. However, the antitumor activity by QTPlus-AM21 was not significantly improved when the dose was increased from 3 mg/kg to 6 mg/kg (FIG. 14). Nonetheless, treatment with QTPlus-AM21 showed increased CD45+ tumor-infiltrated immune cells (FIGS. 15A and 15B) and F4/80+CD86+ M1 populations (FIGS. 15C and 15D). Significant upregulation of Cxel10, Ifna, and Infa was observed in spleens from mice treated with QTPlus-AM21 (FIG. 16A). Il-12 expression was not affected by QTPlus-AM21 in vivo compared with in vitro results (FIG. 12). Pdl1/Pdl upregulation was observed in tumor (FIG. 16B) and spleen (FIG. 16C) tissues from mice treated with QTPlus-AM21.

Combination Therapy of QTPlus-AM21 and Atezolizumab. In MC38 syngeneic mouse model, both QTPlus-AM21 monotherapy and QTPlus-AM21/atezolizumab combination showed significant antitumor responses. Though not statistically significant, the combination therapy of QTPlus-AM21 should synergistic antitumor activity with atezolizumab in MC38 syngeneic mouse model. No significant differences in body weight in mice treated with QTPlus-AM21 and atezolizumab individually or in combination had minor systemic toxicity.

### Discussion

QTPlus as an efficient non-viral gene delivery platform. LNPs have been developed as promising platform to deliver a variety of therapeutic agents. Liposomes were first developed to encapsulate small-molecule chemotherapies as anticancer therapeutics. The benefits of using LNPs to deliver small molecules are to enhance the therapeutic efficacy by increasing the half-life of active compounds in systemic fluids and targeting tumors through EPR effect. To maintain these advantages, the components of traditional LNP for small molecule delivery usually contains certain amounts of PEG lipids to prevent degradation LNP/drug complex in systemic fluids and to reduce particle size for EPR effect. However, these designs for traditional LNPs may not be capable of delivering genetic payloads since gene delivery requires a rapid drug releasement from LNP to cytoplasm to achieve therapeutic gene regulation or expression. Therefore, when applied for gene delivery, LNPs are expected to prevent degradation in systemic fluids but to rapid release their cargos by endosomal escape once they are internalized by cells, suggesting that lower amounts of PEG lipids and more amounts of functional lipids should be considered when designing LNPs for gene delivery.

By lowering the amount of cationic lipids and PEG lipids, QTPlus showed significant increases in mRNA delivery in vitro compared with QTsome Original (FIGS. 1A-IC). Phosphatidylethanolamine (PE) lipids with unsaturated fatty acid chains tend to form inverted hexagonal (Hn) phase which could greatly facilitate endosomal escape. Indeed, QTPlus with DOPE as the helper lipid showed the highest mRNA expression in vitro (FIG. 1E). The gene delivery efficiency by ionizable lipids may vary based on the specific design of different LNPs. Here in the QTPlus platform, A-066 showed the highest mRNA delivery efficiency compared with other commercialized ionizable lipids (FIG. 1D). In terms of formulation procedures, the mixing of empty QTPlus with RNA solution may generate more sealed particle structure to prevent contact between encapsulated RNA and nucleases in the systemic fluids which may be the reason that 2-Step QTPlus-mRNA formulation exhibited higher mRNA expression than 2-Step (FIG. 1F). Lastly, the QTPlus-mRNA product con be developed under room temperature to prevent RNA hydrolysis after heating (FIG. 1G).

In terms of oligonucleotide delivery, QTPlus was also able to deliver AM21. Although QTPlus-AM21 exhibited larger size than QTsome Original-AM21 (FIG. 2A), the particle size within 200 nm is considered suitable for cellular uptake. QTPlus also showed great encapsulation rate for AM21 with N/P ratio from 3 to 10 as demonstrated by gel electrophoresis (FIG. 2C) where the capable N/P ratio of QTPlus could be more tolerable than the current LNP-based vaccines. In general, QTPlus with optimized compositions showed significant increases in both oligonucleotide and mRNA delivery in vitro.

AM-21 as a potent antitumor agent against NSCLC. Although 16-mer and 20-mer AM21 exhibit different miR-21 downstream gene regulation profiles (FIGS. 3A-3B), 16-mer AM21 was superior to 20-mer AM21 in inhibiting tumor cell growth in vitro (FIGS. 4B and 4C). This is because mature miR-21 are often complexed with Ago2 in cytoplasm where the binding domain between miR-21 and Ago2 will be the first and 17th to 21^{st} nucleotides from 5′ end. Therefore, a 16-mer ASO targeting miR-21 would bypass the Ago2 binding domains to achieve fully complementary binding with miR-21 but not 20-mer ASO. The different gene regulation profiles of AM21 in multiple cancer cell lines suggests that NSCLC is the most sensitive to dysregulated miR-21 biological processes compared with other types of tumors (Table 1). Therefore, NSCLC was chosen as the main indication for QTPlus-AM21.

Moreover, QTPlus was able to greatly enhance AM21 delivery compared with AM21 transfection in free solution (FIGS. 6A and 6B), with tumor growth inhibition fully relying on miR-21 inhibition rather than acute cytotoxicity by QTPlus (FIG. 7). Pharmacologically, miR-21 regulation also shares some signaling pathways which could enhance or remedy the therapeutic mechanisms by chemotherapies such as tyrosine kinase inhibitors (TKIs) (30,31). Research showed that treatment with TKIs could recover PTEN expression in lung cancer cells. However, EGFR-mutant lung cancer would develop TKI drug resistance by inhibiting PTEN expression. Here, treatment with QTPlus-AM21 could promote PTEN expression as well as inhibiting EGFR level in A549 TME (FIGS. 8A-8D) which eventually lead to promising antitumor efficacy by QTPlus-AM21 monotherapy and QTPlus-AM21/erlotinib combination therapy (FIG. 10), suggesting that QTPlus-AM21 would be a promising anticancer agent as monotherapy or with combination with first-line TKI-based chemotherapies against NSCLC.

AM-21 turned tumor hot which benefits anti-PDL1 therapy. Although researchers have demonstrated that miR-21 deficiency would lead to macrophage polarization into M1 populations and other antitumor immune responses in transgenic miR-21-depletion mice, there is very few evidence showing that antisense oligonucleotide therapies could also effectively induce two-way therapeutic effects targeting tumor and immune system. Here, the QTPlus-AM21 induced upregulation of CD86 in both human and mouse macrophages (FIGS. 11C and 11D) which was further demonstrated to induce macrophage proliferation and M1 polarization by flow cytometry results (FIGS. 13C and 13D). Moreover, M1 macrophages stimulated by QTPlus-AM21 could inhibit MC38 cancer cell growth and wound-healing (FIG. 12) at the same time when QTPlus-AM21 could direct induce apoptosis in MC38 cancer cell populations (FIGS. 13A and 13B). This effect could be explained by the cytokine- or chemokine-dependent cytotoxicity that the secreted CXCL10, IL-12, and TNFa in QTPlus-AM21-stimulated macrophages could inhibit MC38 cancer cell growth in vitro. Although 3 mg/kg of QTPlus-AM21 showed significant antitumor activity in MC38 syngeneic mouse model, 6 mg/kg of QTPlus-AM21 did not exhibit a significant higher response (FIG. 14). This might be due to the upregulated PD1/PD-L1 expression by QTPlus-AM21 as demonstrated in vitro and in vivo which enhanced immune escape of tumors (FIGS. 11A and 11B, FIGS. 16B and 16C). Nonetheless, QTPlus-AM21 treatment to syngeneic tumor-bearing mice successfully increased CD45+ tumor-infiltrated immune cells and F4/80+CD86+M1 populations in TME (FIGS. 15A-15C), suggesting that QTPlus-AM21 could turn tumor “hot” which would be beneficial for additional immunotherapies. Eventually, QTPlus-AM21 monotherapy and QTPlus-AM21/atezolizumab combination therapy both showed significant antitumor responses in MC38 syngeneic mouse model, suggesting that QTPlus-AM21 would also be a strong anticancer agent as monotherapy against colorectal cancers or with combination with PD1/PD-L1 immune-checkpoint blockade.

### Conclusion

Increasing amount of approved nucleic acid therapeutics has demonstrated the potential to treat diseases by gene regulation in vivo. However, their clinical translation depends on delivery technologies which could improve stability, drug release by endosomal escape and gene regulation profiles. LNPs provide a lipid compartment for nucleic acid cargos which could sequester from serum nuclease activity and facilitate cellular uptake. In the present study, the composition of QTsome was optimized for gene delivery, including oligonucleotides and mRNA. The optimized QTPlus showed significant gene delivery efficiency compared with QTsome Original. AM21 was developed against cancer and was encapsulated into QTPlus to evaluate the antitumor efficacy in vitro and in vivo Pharmacologically, AM21 showed significant miR-21 downstream gene regulation and was able to polarize macrophage into M1 population which in favors of antitumor immune responses. QTPlus-AM21 showed synergistic antitumor activity in vivo in single or combination treatment with erlotinib and atezolizumab which shed light on its great potentials as monotherapy against NSCLC and colorectal cancer and as adjunct therapy with first-line TKI-based chemotherapies and immune-checkpoint blockade

### REFERENCES

1. Shah M Y, Ferrajoli A, Sood A K, Lopez-Berestein G, Calin G A. microRNA Therapeutics in Cancer—An Emerging Concept. EBioMedicine (2016) 12:34-42. doi: 10.1016/j.ebiom.2016.09.017.
2. MacFarlane L-A, R. Murphy P. MicroRNA: Biogenesis, Function and Role in Cancer. Curr Genomics (2010) 11:537-561. doi: 10.2174/138920210793175895.
3 Bartel D P. MicroRNAs. Target Recognition and Regulatory Functions. Cell (2009) 136:215-233. doi: 10.1016/j.cell.2009.01.002.
4. Chevillet J, Lee I, Briggs H, He Y, Wang K. Issues and Prospects of microRNA-Based Biomarkers in Blood and Other Body Fluids. Molecules (2014) 19:6080-6105. doi: 10.3390/molecules 19056080.
5. Condrat C E, Thompson D C, Barbu M G, Bugnar O L, Boboc A, Cretoiu D, Suciu N, Cretoiu S M, Voinea S C. miRNAs as Biomarkers in Disease: Latest Findings Regarding Their Role in Diagnosis and Prognosis. Cells (2020) 9:276. doi: 10.3390/cells9020276.
6. Lawrie C H, Gal S, Dunlop H M, Pushkaran B, Liggins A P, Pulford K, Banham A H, Pezzella F, Boultwood J, Wainscoat J S, et al. Detection of elevated levels of tumour-associated microRNAs in serum of patients with diffuse large B-cell lymphoma. Br J Haematol (2008) 141:672-675. doi: 10.1111/j.1365-2141.2008.07077.x.
7. Wei J, Gao W, Zhu C-J, Liu Y-Q, Mei Z, Cheng T, Shu Y-Q. Identification of plasma microRNA-21 as a biomarker for early detection and chemosensitivity of non-small cell lung cancer. Chin J Cancer (2011) 30:407-414. doi: 10.5732/cjc.010.10522.
8. Peng Q, Zhang X, Min M, Zou L, Shen P, Zhu Y. The clinical role of microRNA-21 as a promising biomarker in the diagnosis and prognosis of colorectal cancer: a systematic review and meta-analysis. Oncotarget (2017) 8:44893-44909. doi: 10.18632/oncotarget. 16488.
9 Xu L, Wu Z. Chen Y, Zhu Q, Hamidi S, Navab R. MicroRNA-21 (miR-21) Regulates Cellular Proliferation, Invasion, Migration, and Apoptosis by Targeting PTEN, RECK and Bcl-2 in Lung Squamous Carcinoma, Gejiu City, China. PLOS One (2014) 9: e103698. doi: 10.1371/journal.pone.0103698.
10. Xiao L, He Y, Peng F, Yang J, Yuan C. Endometrial Cancer Cells Promote M2-Like Macrophage Polarization by Delivering Exosomal miRNA-21 under Hypoxia Condition. J Immunol Res (2020) 2020:1-9. doi: 10.1155/2020/9731049.
11. Xi J, Huang Q, Wang L, Ma X, Deng Q, Kumar M, Zhou Z, Li L, Zeng Z, Young K H, et al. miR-21 depletion in macrophages promotes tumoricidal polarization and enhances PD-1 immunotherapy. Oncogene (2018) 37:3151-3165. doi: 10.1038/s41388-018-0178-3.
12. Di Fusco D, Dinallo V, Marafini I, Figliuzzi M M, Romano B, Monteleone G. Antisense Oligonucleotide: Basic Concepts and Therapeutic Application in Inflammatory Bowel Disease. Front Pharmacol (2019) 10: doi: 10.3389/fphar.2019.00305.
13. Castanotto D, Stein C A. Antisense oligonucleotides in cancer. Curr Opin Oncol (2014) 26:584-589. doi: 10.1097/CCO.0000000000000127.
14. Roberts T C, Langer R, Wood M J A. Advances in oligonucleotide drug delivery. Nat Rev Drug Discov (2020) 19:673-694. doi: 10.1038/s41573-020-0075-7.
15. Yung B C, Li J, Zhang M, Cheng X, Li H, Yung E M, Kang C, Cosby L E, Liu Y, Teng L, et al. Lipid Nanoparticles Composed of Quaternary Amine-Tertiary Amine Cationic Lipid Combination (QTsome) for Therapeutic Delivery of AntimiR-21 for Lung Cancer. Mol Pharm (2016) 13:653-662. doi: 10.1021/acs.molpharmaceut.5b00878.
16. Yu B, Wang X, Zhou C, Teng L, Ren W, Yang Z, Shih C-H, Wang T, Lee R J, Tang S, et al. Insight into Mechanisms of Cellular Uptake of Lipid Nanoparticles and Intracellular Release of Small RNAs. Pharm Res (2014) 31:2685-2695. doi: 10.1007/s11095-014-1366-7.
17. Schoenmaker L, Witzigmann D, Kulkarni J A, Verbeke R, Kersten G, Jiskoot W, Crommelin D JA. mRNA-lipid nanoparticle COVID-19 vaccines: Structure and stability. Int J Pharm (2021) 601:120586. doi: 10.1016/j.ijpharm.2021.120586.
18. OSTRO MJ, GIACOMONI D, LAVELLE D, PAXTON W, DRAY S. Evidence for translation of rabbit globin mRNA after liposomemediated insertion into a human cell line. Nature (1978) 274:921-923. doi: 10.1038/274921a0.
19. Bangham A D, Horne R W. Negative staining of phospholipids and their structural modification by surface-active agents as observed in the electron microscope. J Mol Biol (1964) 8:660-IN10. doi: 10.1016/S0022-2836 (64) 80115-7.
20. Sharma V K, Agrawal M K. A historical perspective of liposomes-a bio nanomaterial. Mater Today Proc (2021) 45:2963-2966. doi: 10.1016/j.matpr.2020.11.952.
21. Milla P, Dosio F, Cattel L. PEGylation of Proteins and Liposomes: A Powerful and Flexible Strategy to Improve the Drug Delivery. Curr Drug Metab (2012) 13:105-119. doi: 10.2174/138920012798356934.
22. Wu J. The Enhanced Permeability and Retention (EPR) Effect: The Significance of the Concept and Methods to Enhance Its Application. J Pers Med (2021) 11:771. doi: 10.3390/jpm11080771.
23. Zhao Y, Huang L. “Lipid Nanoparticles for Gene Delivery.,” (2014). p. 13-36 doi: 10.1016/B978-O-12-800148-6.00002-X.
24. Schlich M, Palomba R, Costabile G, Mizrahy S, Pannuzzo M, Peer D, Decuzzi P. Cytosolic delivery of nucleic acids: The case of ionizable lipid nanoparticles. Bioeng Transl Med (2021) 6: doi: 10.1002/btm2.10213.
25. Zhang C, Ma Y. Zhang J, Kuo J C-T, Zhang Z, Xie H, Zhu J, Liu T. Modification of Lipid-Based Nanoparticles: An Efficient Delivery System for Nucleic Acid-Based Immunotherapy. Molecules (2022) 27:1943. doi: 10.3390/molecules27061943.
26. Tenhunen J. Hydrolysis of single-stranded RNA in aqueous solutions-effect on quantitative hybridizations. Mol Cell Probes (1989) 3:391-396. doi: 10.1016/0890-8508 (89) 90018-2.
27. Sabourian P, Yazdani G, Ashraf S S, Frounchi M, Mashayekhan S, Kiani S, Kakkar A. Effect of Physico-Chemical Properties of Nanoparticles on Their Intracellular Uptake. Int J Mol Sci (2020) 21:8019. doi: 10.3390/ijms21218019.
28. Baranov M V., Kumar M, Sacanna S, Thutupalli S, van den Bogaart G. Modulation of Immune Responses by Particle Size and Shape. Front Immunol (2021) 11: doi: 10.3389/fimmu.2020.607945.
29. Angart P, Vocelle D, Chan C, Walton S. Design of siRNA Therapeutics from the Molecular Scale. Pharmaceuticals (2013) 6:440-468. doi: 10.3390/ph6040440.
30. Zhang W C, Slack F J. MicroRNA-21 Mediates Resistance to EGFR Tyrosine Kinase Inhibitors in Lung Cancer. J Thorac Oncol (2017) 12: S1536. doi: 10.1016/j.jtho.2017.06.021.
31. Li B. Ren S, Li X, Wang Y, Garfield D, Zhou S, Chen X, Su C, Chen M, Kuang P, et al. MiR-21 overexpression is associated with acquired resistance of EGFR-TKI in non-small cell lung cancer. Lung Cancer (2014) 83:146-153. doi: 10.1016/j.Jungcan.2013.11.003.
32. Noro R, Gemma A, Miyanaga A, Kosaihira S, Minegishi Y, Nara M, Kokubo Y, Seike M, Kataoka K, Matsuda K, et al. PTEN inactivation in lung cancer cells and the effect of its recovery on treatment with epidermal growth factor receptor tyrosine kinase inhibitors. Int J Oncol (2007) 31:1157-63.
33. Sos M L, Koker M, Weir B A, Heynck S, Rabinovsky R, Zander T, Seeger J M, Weiss J, Fischer F, Frommolt P, et al. PTEN Loss Contributes to Erlotinib Resistance in EGFR-Mutant Lung Cancer by Activation of Akt and EGFR. Cancer Res (2009) 69:3256-3261.

doi: 10.1158/0008-5472.CAN-08-4055.
34. Sahraei M, Chaube B, Liu Y, Sun J, Kaplan A, Price N L, Ding W, Oyaghire S, Garcia-Milian R, Mehta S, et al. Suppressing miR-21 activity in tumor-associated macrophages. promotes an antitumor immune response. J Clin Invest (2019) 129:5518-5536. doi: 10.1172/JCI127125.
35. Muroya M, Chang K, Uchida K, Bougaki M, Yamada Y. Analysis of cytotoxicity induced by proinflammatory cytokines in the human alveolar epithelial cell line AS49. Biosci Trends (2012) 6:70-80.
36. Jiang X, Wang J, Deng X, Xiong F, Ge J, Xiang B, Wu X, Ma J, Zhou M, Li X, et al. Role of the tumor microenvironment in PD-L1/PD-1-mediated tumor immune escape. Mol Cancer (2019) 18:10. doi: 10.1186/s12943-018-0928-4.
37. Garrido-Martin E M, Mellows T WP, Clarke J, Ganesan A-P, Wood O, Cazaly A, Seumois G, Chee S J, Alzetani A, King E V, et al. M1 hot tumor-associated macrophages boost tissue-resident memory T cells infiltration and survival in human lung cancer. J Immunother Cancer (2020) 8: e000778. doi: 10.1136/jitc-2020-000778.

### Example 2. Novel Compositions of Lipid Nanoparticles for Intramuscular Delivery of mRNA with Reduced Systemic Gene Expression

Severe acute respiratory syndrome coronavirus 2 (SARS-COV-2) is known as the pathogen which causes the coronavirus disease 2019 (COVID-19). Coronaviruses are single-stranded RNA viruses coated by the lipid membrane containing envelope (E), membrane (M) and spike(S) proteins. During the recent two years, the outbreak of COVID-19 pandemic has had major influence on the whole world. By November 2021, there are nearly 0.25 billion coronavirus cases in the world, of which about 5 million deaths. The high morbidity and mortality of SARS-COV-2 promotes the need of effective vaccines. In 2020, two mRNA vaccine candidates, mRNA-1273 and BNT162b2, were developed for the defense of COVID-19 by Moderna and Pfizer/BioNTech respectively. Messenger RNAs (mRNAs) are a type of single-stranded molecule of nucleic acids transcribed from DNA. mRNAs play an essential role in transferring genetic message from the nuclei to the ribosomes where the proteins are synthesized. mRNAs as vaccines have an advantage that they can encode any proteins while they do not have to be delivered to the nuclei. The major mRNA vaccine candidates encode S protein to activate antibody response against the trimeric S protein of SARS-COV-2. While the mRNA therapeutics are promising treatment strategies in many diseases, the pitfalls have limited their development First, mRNA is easily degraded by RNase in the plasma and has short circulation time without protection. Also, negative charge of mRNA obstructs it go through the negatively charged cell membrane, and in further target to the cytosol. In some situations mRNA may also cause activation of undesired immune response. Lipid nanoparticles (LNPs) have enabled efficient delivery of mRNA. LNPs containing ionizable lipids, of which the head group is positively charged, can encapsulate nucleic acids efficiently and sufficiently via self-assembly process. mRNAs will then be trapped in the inside aqueous phase and protected by LNP from RNase and macrophage. Generally, a LNP prepared for mRNA delivery consists of a helper lipid, an ionizable lipid, cholesterol, and polyethylene glycol (PEG) to form a stable formulation with relatively long circulation time. For instance, mRNA-1273 is encapsulated by Distearoylphosphatidylcholine (DSPC), cholesterol, methoxypoly(ethylene glycol) dimyristoyl glycerol (PEG-DMG), and SM-102, a high-performance pH-responsive ionizable lipid of which the patent belongs to Moderna.

While mRNA vaccines from Pfizer/BioNTech and Moderna have been revolutionary in terms of their impact on public health during the COVID-19 pandemic, there have been incidences of severe adverse reactions. It is reported that current SARS-CoV-2 mRNA vaccine may cause severe myopericarditis. While exceedingly rare, these adverse reactions have a common reason cited for vaccine hesitancy. A possible cause of the adverse reaction is systemic gene expression, which can be reduced if mRNA delivery can be limited to the muscle, in other word, the local site of administration. A series of novel compositions of lipid nanoparticles for mRNA vaccines injected by IM called QTsome are described in this Example. QTsomes are LNPs that incorporate a combination of quaternary (cationic) and tertiary amine (ionizable) lipids. They can further include other lipids. In some examples, the QTsomes can be formed from a helper lipid, cholesterol, PEG-DMG, an ionizable lipid and 1,2-dioleoyl-3-trimethylammonium-propane (DOTAP) which is a cationic lipid carrying positive charge permanently. By introducing a cationic lipid in the LNP, QTsomes are expected to perform efficient mRNA delivery by IM injection and reduced systemic gene expression since most of mRNA will be restricted in the muscle.

### Materials and Methods

DSPC, DOPC, DOPE, DOTAP and cholesterol were purchased from Avanti Polar Lipids (Alabaster, AL). DODMA and DMG-PEG2000 were purchased from NOF America (White Plains, NY). SM-102 was synthesized by DC Chemical (Shanghai, China). A066 was synthesized by Dragon Pharma (Shanghai, China). Firefly luciferase messenger RNA (FFLuc mRNA) was purchased from TriLink Biotechnologies (San Diego, CA). Any chemicals or buffers otherwise stated were purchased from Fisher Scientific (Hampton, NH).

Helper lipids (including DSPC, DOPC and DOPE), DOTAP, ionizable lipids (including SM-102, A066 and DODMA), cholesterol, DMG-mPEG2000 were mixed into a lipid ethanol mixture at a molar ratio of 12:3-8:45-40:38.5:1.5. The lipid-ethanol mixture was diluted to a final lipid concentration of 8 mg/mL. Messenger RNA was diluted in 25 mM citrate acidic buffer, pH 2.75, at a concentration of 0.133 mg/mL. mRNA lipid QTsome+ nanoparticle was formulated by injecting 250 μL of lipid ethanol solution into 750 μL of mRNA/sodium citrate solution on a vortex at room temperature using a 29G-½″ insulin syringe. The solution was mixed well on slow vortex for 15 minutes. Next, 100 μL of 0.2M Tris-HCl solution (pH 8.0) was added to the lipid QTsome+ nanoparticle solution, and the pH was titrated to pH 7.4 using 2.0M sodium hydroxide solution. The pH was checked by pH papers. To finalize the product, 1000 μL of 20 mM Tris-HCl solution (pH 7.4) was added and the final solution was dialyzed in PBS overnight at 4° C.

The particle sizes and zeta potential (C) of nucleic acid-loaded pH-sensitive nanoemulsion were analyzed by dynamic light scattering with a NICOMP Z3000 Nano DLS/ZLS Systems (Entegris, Billerica, MA). The mRNA concentrations were measured by Quant-it™ RiboGreen RNA Assay Kit.

ICR CD-1 Swiss mice were purchased from Charles River Laboratory Animals were housed in a temperature-controlled room under a 12 hr light/12 hr dark cycle and fed normal chow diet. All animal studies were reviewed and approved by the Ohio State University Institutional Laboratory Animal Care and Use Committee. Both male and female mice were used for experiments.

In vivo mRNA expression was quantified by measuring firefly luciferase bioluminescence. Mice treated with IM injection of FFLuc mRNA lipid QTsome+ nanoparticles at 1.5 μg per leg were injected with luciferin intraperitonially 5 minutes prior to bioluminescence measurements. Bioluminescence images were taken by a in vivo imaging system (IVIS) under optimal exposure settings.

To evaluate the apparent pKa of the lipid QTsome+ nanoparticles including different combinations of lipids, 2-(p-toluidino) naphthalene-6-sulfonic acid (TNS) fluorescence assay was designed. TNS binds to positively charged lipids through electrostatic interactions and emits strong fluorescence upon excitation. A series of universal buffers (a combination of 10 mM NaCitrate, 10 mM NaPhasphate, 10 mM NaBorate, and 150 mM NaCl) with pH ranging from 3 to 12 was prepared. QTsome+ were diluted in these buffers at the final ionizable lipid (including SM-102, A066, DODMA and DOTAP) concentration of 75 μM. TNS was then added to the final concentration at 6.0 μM. pH was measured after thorough mixing of TNS and QTsome+ using a pH meter. TNS Fluorescence was obtained by transferring 200 μL of samples into a black-opaque plate in triplicate, and reading using a SpectraMax M5 plate reader with the excitation (λₑₓ) at 325 nm and the emission (Nem) at 435 nm. It was assumed that in the presence of amino lipids, TNS fluorescence reaches a maximum when 100% of the amino lipids are ionized, while TNS has little fluorescence when the amino lipids are in the un-ionized state. Apparent pKa of the QTsome+ lipid nanoparticles could be elucidated by the following equation after fitting the data points with a three-parameter sigmoid function, with FL against pH:

$$FL = \frac{{FL}_{Max}}{1 - {e}^{\frac{pH - pKa}{b}}}$$

Where FL is TNS fluorescence value, pKa is apparent pKa of the amino lipids, b is the measure of the cooperativeness of the protonation process of amino acids.

### Hypothesis and Key Findings

Adding a quaternary amine (permanently charged cationic lipids) limits liver penetration and lowers systemic toxicity. This provides a compelling reason to select the QTsomes described herein as the delivery platform

### Results

To examine the delivery efficiency of QTsome+ lipid nanoparticles, FFLuc mRNA was selected as the reporter gene of interest to be encapsulated into different QTsome+ formulations. Bioluminescence intensity was measured by Bright-Glo luciferase kit used as the quantification of expressed firefly luciferase after mRNA transfection. We treated 10,000 HEK293T cells overnight with 100 ng FFLuc mRNA utilizing different formulations with lipofectamine 3000 as the positive control. Here, we showed that bioluminescence intensity significantly decreased when the mRNA lipid nanoparticles were formulated at high temperatures. mRNA lipid nanoparticles had the highest bioluminescence intensity while formulating at room temperature. Therefore, the follow-on samples were formulated at room temperature instead of 65° C. or 37° C.

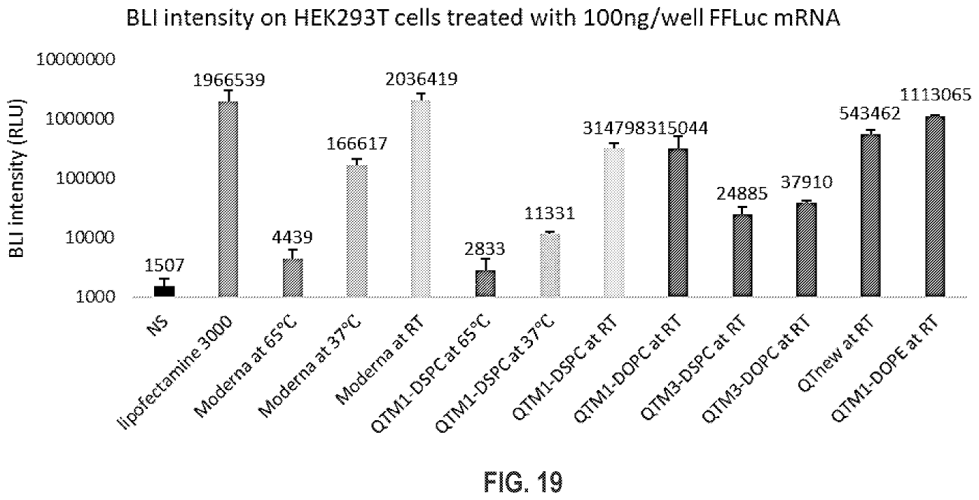

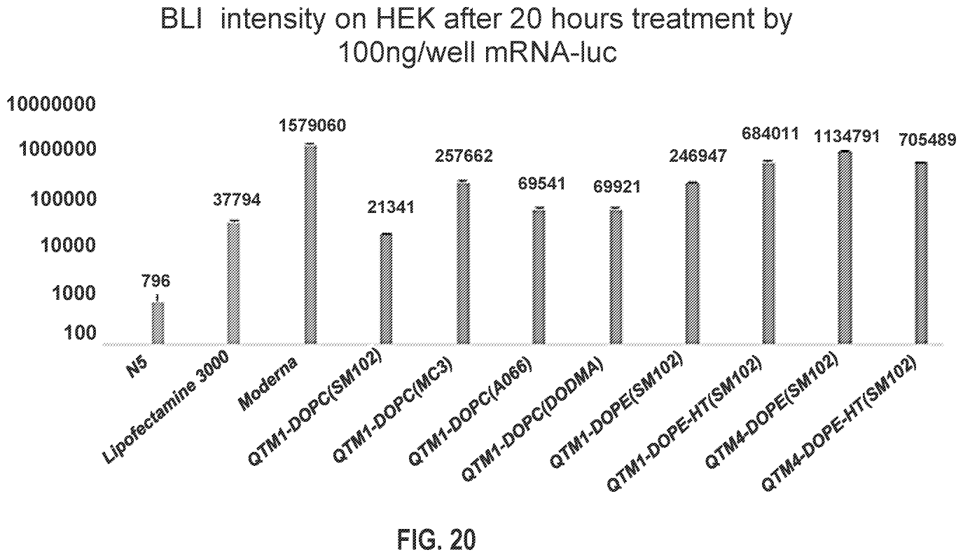

We then compared the delivery efficiency of different helper lipids including DSPC, DOPC and DOPE. Here we demonstrated that DOPE outperformed DOPC and DSPC in vitro using QTsome+ lipid nanoparticles in delivering mRNA into cells. We also compared the delivery efficiency of QTsome+ lipid nanoparticles containing different DOTAP percentages. We fixed the overall charged/chargeable lipids composition at 48%. Here, the result demonstrated that the bioluminescence intensity decreased as DOTAP percentage increased See FIGS. 19 and 20.

To further explore the options for QTsome+ lipid nanoparticles, we further decreased the DOTAP percentages within the formulations as well as changing the ionizable lipids to industry standard DLin-MC3-DMA and patent-free A066. The bioluminescence results suggested that switching to MC3 slightly increased the delivery efficiency, compared to SM-102. Also, further decreasing DOTAP to 1.5% or increasing ionizable lipids to 50% facilitated the delivery processes in vitro.

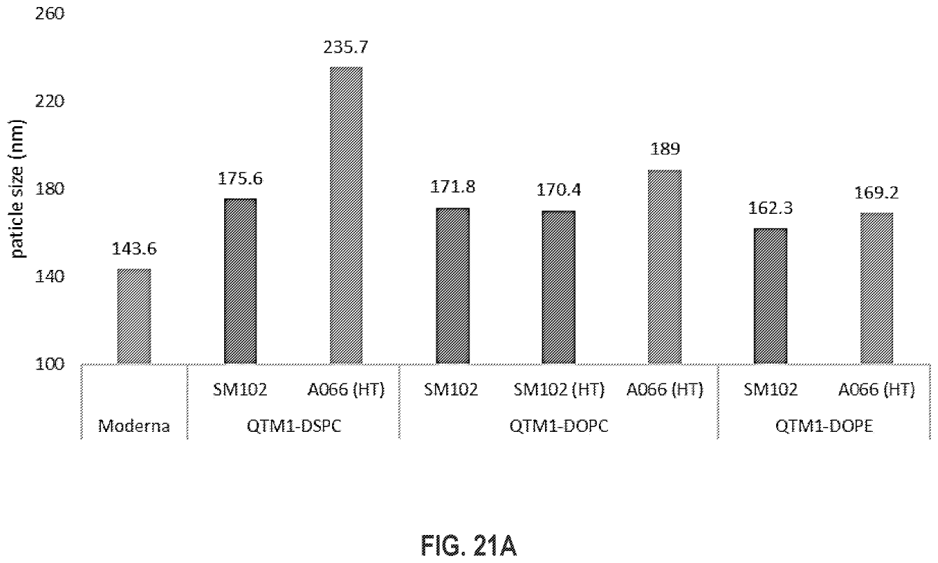

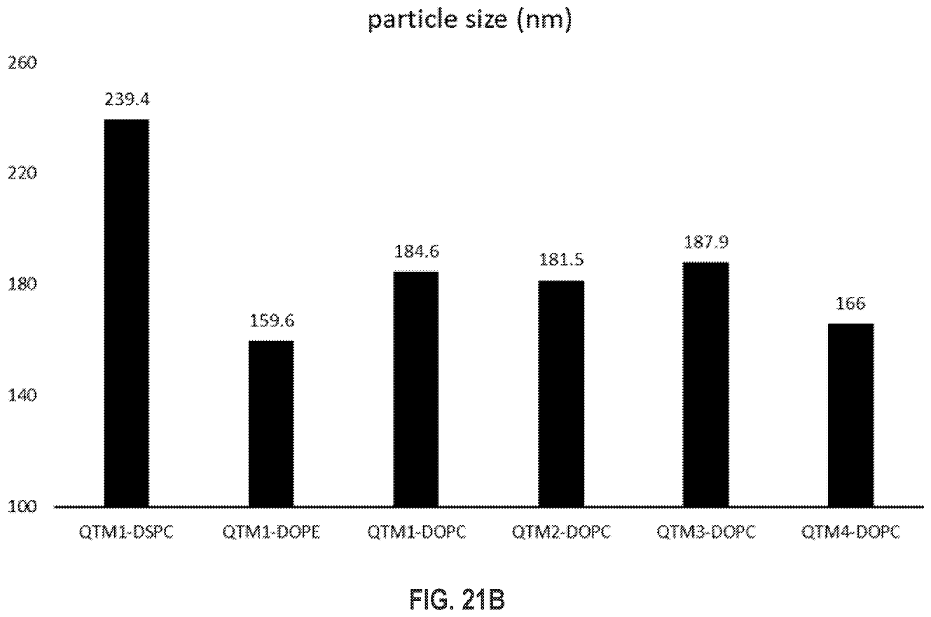

Compared to SM102, a QTsome including A066 has larger particle size, which may result from the different structure of A066, which has short, branched tails that can encapsulate nucleic acids with small molecule weight such as siRNA and miRNA more efficiently, but not as good as SM102 when encapsulating large nucleic acids like mRNA. See FIGS. 21A-21B.

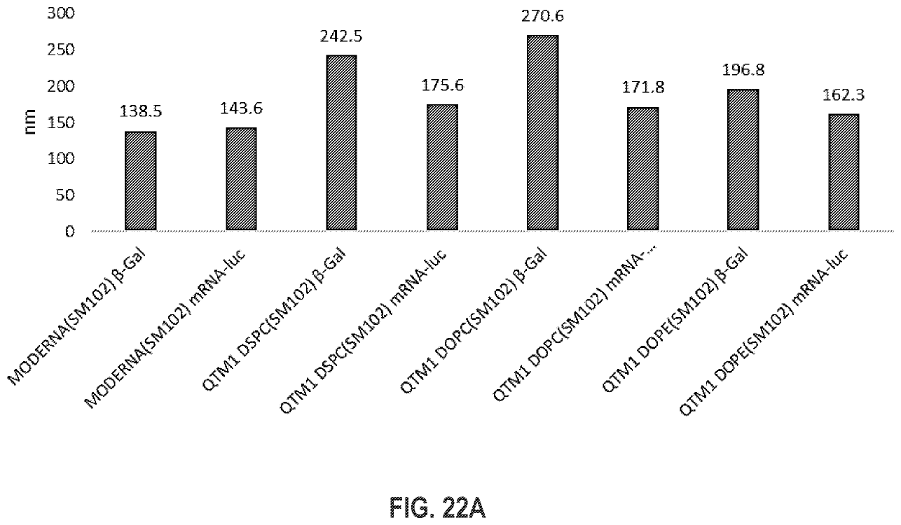

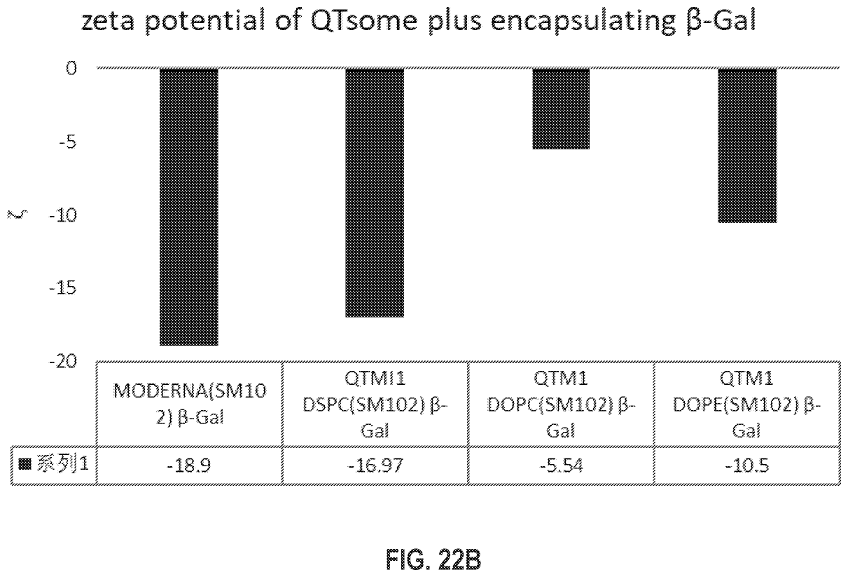

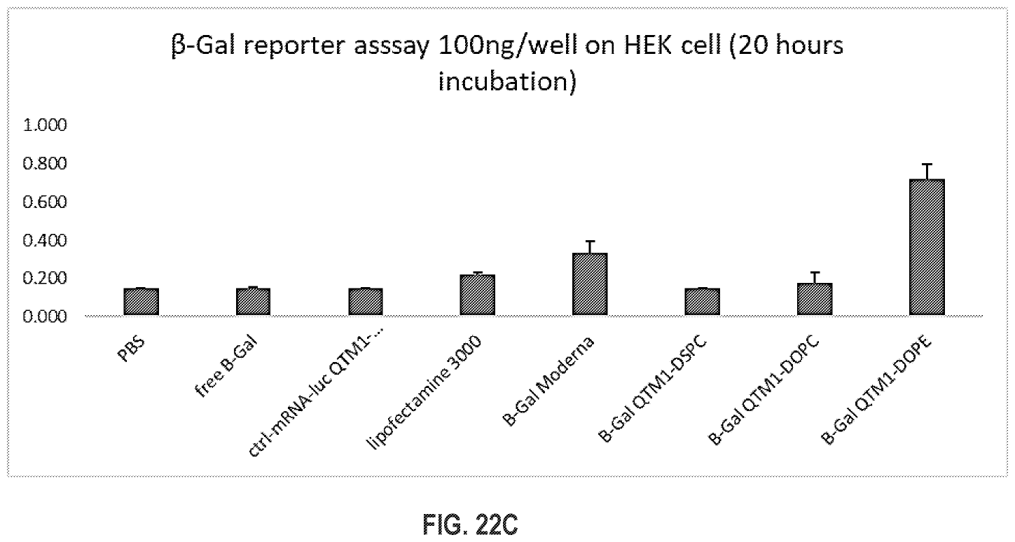

When encapsulating β-Gal, the particle size of QTsome including SM102 is larger than the same QTsome formulations encapsulating mRNA-luc, that because the molecule weight of β-Gal is larger than mRNA-luc. 100 ul samples was diluted in 3 ml distilled water respectively. Compared to other formulation, the absolute value of zeta potential of Qtsome including DOPC was smaller than other formulations, which leads to a larger particle size. Based on the results from β-Gal reporter asssay 100 ng/well on HEK cell, QTM1-DOPE formulation performed best, while Moderna formulation was not as good as QTM1-DOPE but better than Qtsome plus including DOPC and DSPC. See FIGS. 22A-22C.

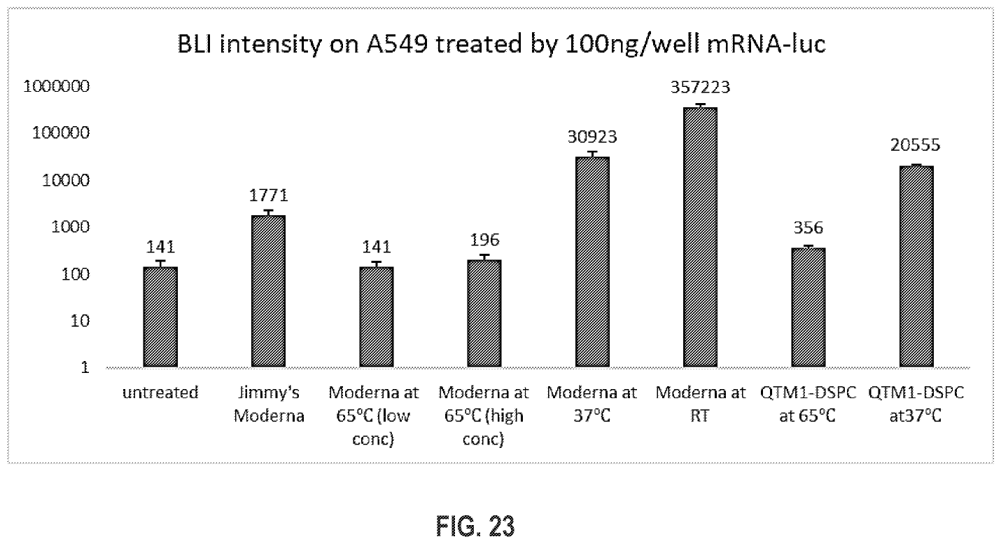

To evaluate the effect of preparation temperature on the transfection efficiency, several mRNA-luc LNPs were prepared at different temperature. Each group was incubated at 37 degree for 20 hours after treating with 100 ng mRNA-luc LNPs on A549 cell line. The transfection efficiency increased significantly with the decrease of temperature. LNPs prepared at room temperature had most efficient transfection. See FIG. 23.

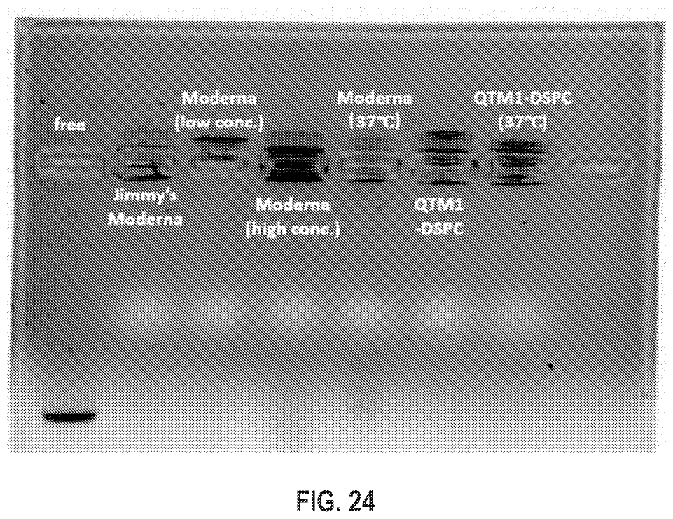

According to the gel electrophoresis image (see FIG. 24), we can conclude that FFLuc mRNAs were highly encapsulated within QTsome+ lipid nanoparticles.

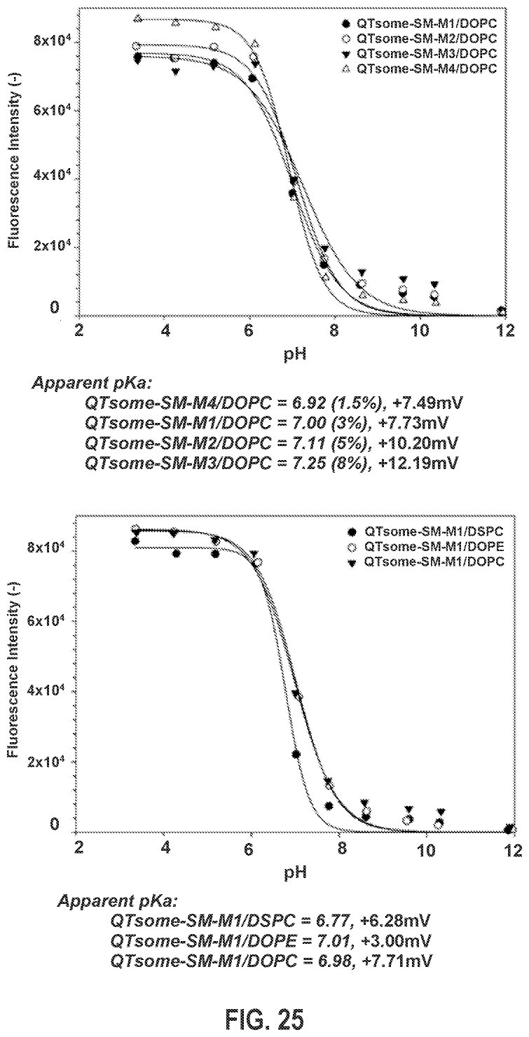

Based on TNS assay results (FIG. 25), the apparent pKa (fitting data from TNS assay) of QTsome+ was highly correlated with the % of quaternary amine, which matched our hypothesis in adding DOTAP as the agent to tune zeta potential as well as apparent pKa of a specific ionizable lipid. DOTAP can be viewed as an ionizable lipid with infinite pKa (which does not release proton but has sufficient positive charge to support membrane disruption).

Interestingly, helper lipids play an important role in modulating apparent pKa. Under the same % of DOTAP, DSPC gave the lowest pKa among all, which reflected in the in vivo delivery efficiency (higher FFLuc expression). DOPC and DOPE had similar pKa, and the in vivo delivery efficiency was majorly managed by the fusion ability of DOPE.

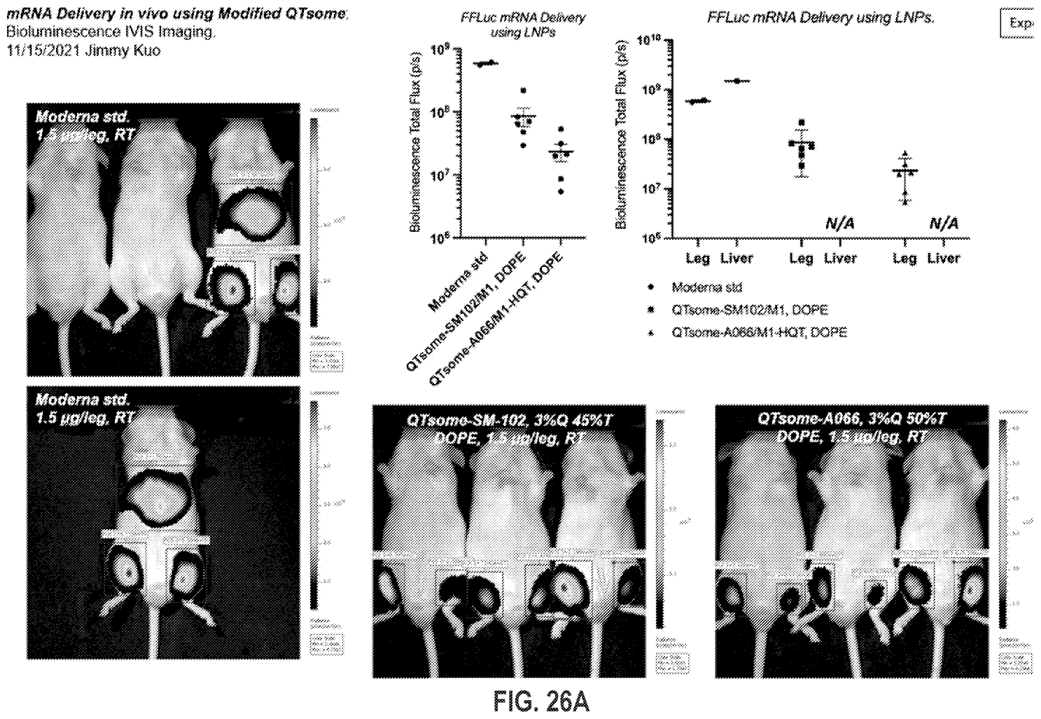

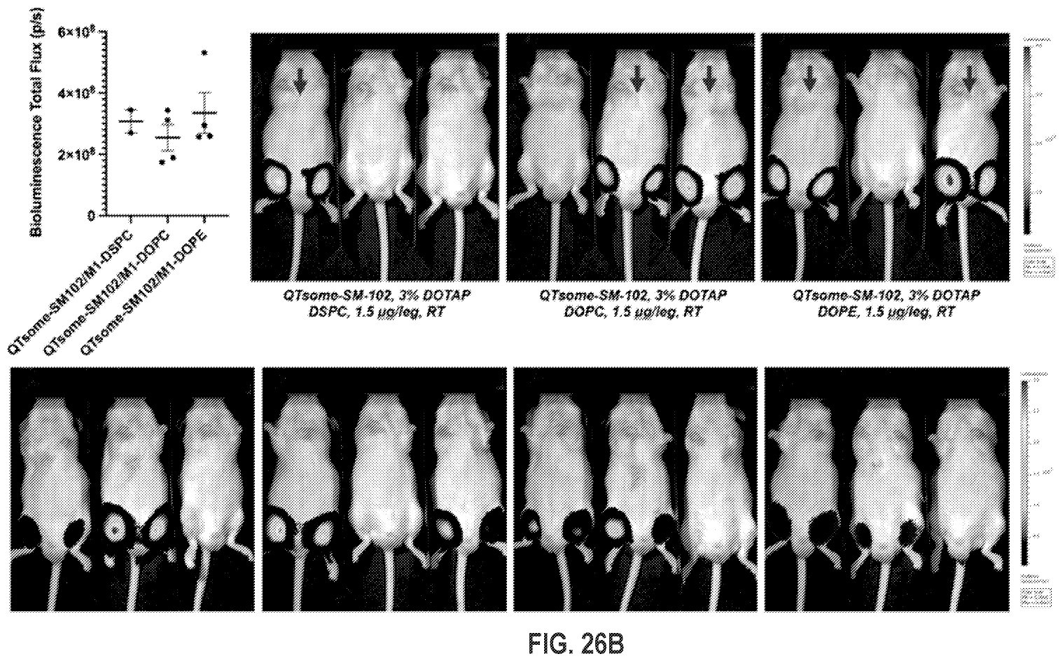

It's worth comparing DSPC+1.5% DOTAP and DOPE+1.5% DOTAP to see which gives the better delivery efficiency. See FIGS. 26A-26B.

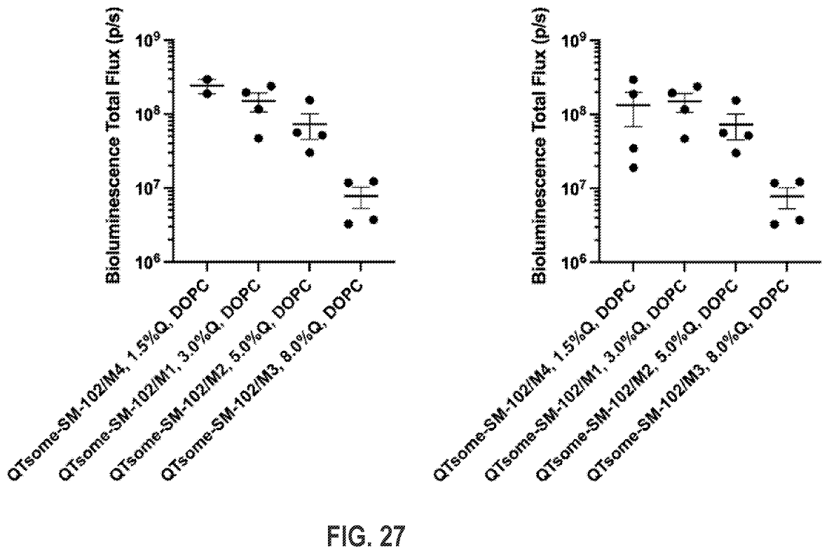

The in vivo injection results showed that there was no significant difference between DSPC, DOPC, or DOPE in FLuc expression. Another run of injections will be done to increase the N value for statistics. See FIG. 27.

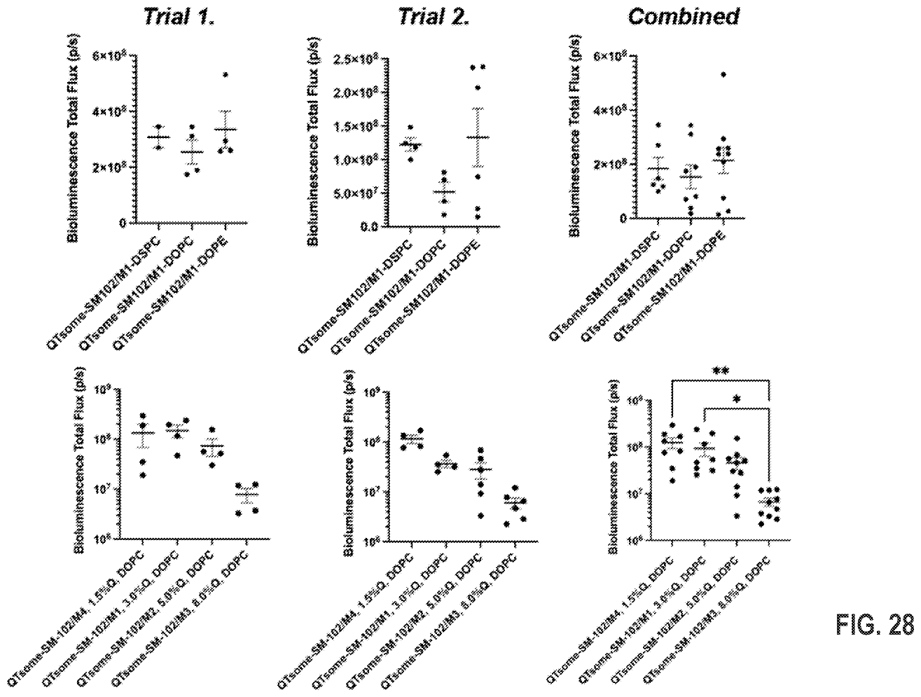

The in vivo injection results showed that there was a reverse trend of FLuc expression versus DOTAP percentages. Q+T percentage of all the samples were fixed at 48%, and DOTAP percentage was the parameter of interest in this experiment. However, by one-way ANOVA, there was no significant difference between each group (left figure). If we discarded two low-expressed samples (marked in the orange arrow), the trend was more obvious. One-way ANOVA significances were shown between 1.5 vs. 5.0, 1.5 vs. 8.0, and 3.0 vs. 8.0. See FIG. 28.

By combining two rounds of IM injections, the in vivo results showed that there were no significant differences between different helper lipids. However, DOPE still rather outperformed PC-based helper lipids. We also showed that by increasing DOTAP percentages within the formulation, the in vivo delivery efficiency decreased, which matched the in vitro experimental results.

### Discussion

Lipid nanoparticles (LNPs) have been developed for several decades for the delivery of nucleic acids since they can protect nucleic acids from degraded by nuclease and eliminated from the circulation. mRNA LNPs including an ionizable lipid, such as mRNA-1273, can help reduce the zeta potential of mRNA LNP and subsequently decrease the cytotoxicity. However, it has been found that there was liver penetration of mRNA after IV or IM injection, which in further may cause systemic toxicity. In this Example, a strategy has been found to limit the liver penetration of mRNA to lower the systemic toxicity by adding additional cationic lipids containing quaternary amines like 1,2-Dioleoyl-3-trimethylammonium propane (DOTAP) or 1,2-Dioleoyl-3-trimethylammonium propane (DOTMA), which carry positive charge permanently. Based on this hypothesis, we propose this advanced nucleic acids delivery system QTsome as the preferred platform for mRNA vaccine formulation.

### REFERENCES

1 Wu, D., Wu, T., Liu, Q. & Yang, Z. The SARS-COV-2 outbreak: What we know. Int. J. Infect. Dis. 94, 44-48 (2020).
2. World Health Organization. WHO Coronavirus (COVID-19) Dashboard.
3. Jackson, L. A. et al. An mRNA Vaccine against SARS-COV-2-Preliminary Report. N. Engl. J. Med. 383, 1920-1931 (2020).
4. Walsh, E. E. et al. Safety and Immunogenicity of Two RNA-Based Covid-19 Vaccine Candidates. N. Engl. J. Med. 383, 2439-2450 (2020).
5. Polack, F. P. et al. Safety and Efficacy of the BNT162b2 mRNA Covid-19 Vaccine. N. Engl. J. Med. 383, 2603-2615 (2020).
6. Hoffmann, M. et al. SARS-COV-2 Cell Entry Depends on ACE2 and TMPRSS2 and Is Blocked by a Clinically Proven Protease Inhibitor. Cell 181, 271-280.e8 (2020).
7. Kaczmarek, J. C., Kowalski, P. S. & Anderson, D. G. Advances in the delivery of RNA therapeutics: from concept to clinical reality. Genome Med. 9, 60 (2017).
8. Klugar, M. et al. Side Effects of mRNA-Based and Viral Vector-Based COVID-19 Vaccines among German Healthcare Workers. Biology (Basel). 10, 752 (2021).
9. Kadali, R. A. K., Janagama, R., Peruru, S. & Malayala, S. V. Side effects of BNT162b2 mRNA COVID-19 vaccine: A randomized, cross-sectional study with detailed self-reported symptoms from healthcare workers. Int. J. Infect. Dis. 106, 376-381 (2021).
10. Ossato, A. et al. Comparison of medium-term adverse reactions induced by the first and second dose of mRNA BNT162b2 (Comimaty, Pfizer-BioNTech) vaccine: a post-marketing Italian study conducted between 1 Jan. and 28 Feb. 2021. Eur. J. Hosp. Pharm. ejhpharm-2021-002933 (2021) doi: 10.1136/ejhpharm-2021-002933.
11. Li, C. et al. Intravenous Injection of Coronavirus Disease 2019 (COVID-19) mRNA Vaccine Can Induce Acute Myopericarditis in Mouse Model. Clin. Infect. Dis. (2021) doi: 10.1093/cid/ciab707.
12. Wayment-Steele, H. K. et al. Theoretical basis for stabilizing messenger RNA through secondary structure design. Nucleic Acids Res. 49, 10604-10617 (2021).
13. Zourabian, R., Votruba, J. & Chaloupka, J. Effect of temperature on translation of mRNA coding for an extracellular proteinase and cell proteins in Bacillus megaterium. Curr. Microbiol. 24, 337-342 (1992).

### Example 3. SiRNA Encapsulated in Lipid Nanoparticles Downregulates Akt1 and Combination Therapy with Lenvatinib for the Treatment of Hepatocellular Carcinoma

As the third most common cause of cancer death, hepatocellular carcinoma (HCC) leaded to about 800,000 deaths in 2020. Hepatocellular carcinoma is the main type of liver cancer, which accounts for about 90% cases of liver cancer. Orthotopic liver transplantation (OLT) is considered to be one of the optimum strategies for the treatment of hepatocellular carcinoma. However, most of patients suffering from early-stage HCC cannot receive OLT in time. With the development of diagnosis and therapy technology, HCC patients are more likely to be cured at earlier stages. Since abnormal gene expression resulting in uncontrolled proliferation of hepatocytes, which subsequently causes the HCC formation, systemic targeted therapies such as sorafenib have been applied for the regulation of oncogene-relevant pathways. To adequately suppress the abnormal growth of cancer cell and achieve expected therapeutic effect, targeting more than one pathway is helpful.

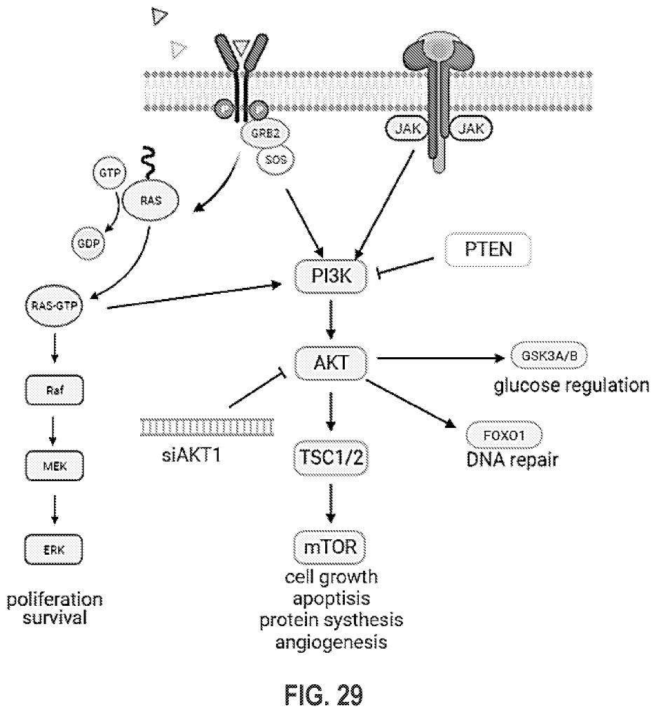

A small interfering RNA (siRNA) specifically targeting to Akt1 was designed in this study to silence the Akt1 mRNA expression and therefore downregulate protein translation, as well as influence multiple downstream pathways. Akt1 plays an essential role in cell migration proliferation, and apoptosis. It has been believed to regulate anti-apoptosis in many cell death paradigms. Downregulation of Akt1 gene expression will suppress downstream signaling and promote cell death happening on cancer cells (FIG. 29). The siRNA was encapsulated in an advanced drug delivery system consisting of cationic lipid and ionizable lipid simultaneously called QTsome plus, which is based on previous QTsome version designed by Yung et al in 20166 Generally nucleic acids such as siRNA and miRNA are delivered by lipid nanoparticles consisting of ionizable lipids instead of cationic lipids to avoid cytotoxicity. However, the ionizable lipids is neutral at physiological condition, the surface of lipid nanoparticles with no charge cannot taken up by cells easily. To enhance the delivery efficiency of siRNA encapsulated in this study, QTsome plus consisting of both quaternary ammonium and tertiary amine simultaneously was designed. The quaternary ammonium is permanently positively charged, of which the ratio is much lower than pH-sensitive tertiary amine, therefore the lipid nanoparticles will not aggregate and can fuse with negatively charged membrane at physiological pH condition while it will not cause severe cytotoxicity at the same time.

To enhance the anti-tumor effect on HCC model, a recently FDA-approved drug Lenvatinib was also used as a combination therapy with siRNA-Akt1 QTsome plus. Lenvatinib works as a kinase inhibitor against multiple kinase including vascular endothelial growth factor receptors (VEGFR) 1-3 and fibroblast growth factor receptors (FGFR) 1-47. Lenvatinib has shown anti-tumor activity against multiple cancers including melanoma, thyroid cancer, and hepatocellular carcinoma^{8,9}. The objective of this study is to evaluate the anti-tumor effect of siRNA targeting Akt1 encapsulated by novel vehicle and the combination therapy of siRNA and Lenvatinib on HCC cell line-derived xenograft (CDX) model.

### Materials and Methods

Materials. Cholesterol and PEG-DMG were purchased from Avanti® Polar Lipids. Lipids were purchased from MedChemExpress. Primers for RT-qPCR were purchased from ThermoFisher®. SYBR Green SuperMix was purchased from BioRad. Applied Biosystem™ high-capacity cDNA reverse transcription kits were purchased from FisherSci. Primary and secondary antibodies for western blot were purchased from Cell Signaling Technologies (CST) except antibody for human FRS2, which was purchased from R&D system. Mini-protein TGX Gels, Tris/Glycine were purchased from BIO-RAD. PI/RNase Staining Buffer, BD Annexin V-FITC, and RNase A were purchased from FisherSci. siRNA sequences were synthesized by Integrated DNA Technologies (IDT). Lenvatinib was purchased from Caymen Chemical. Peripheral blood mononuclear cells (PBMCs) were purchased from IQ Biosciences Human ELISA kits for IL-6, TNF-α, and IFN-γ were purchased from ThermoFisher.

Preparation of siRNA-Encapsulated Lipid Nanoparticle Formulation. Mix the stock solution of lipids, cholesterol and PEG-DMG together and inject it into 25 mM citrate RNase-free water. Sonicate the empty lipid nanoparticle and then filter with 0.45 nm membrane in sterile. Add siRNA to empty lipid nanoparticle with drug lipid ratio is 1:12. After that, process dialysis to remove ethanol and adjust pH.

Cells and cell culture. Cell lines were purchased from JCRB cell bank. Huh7 was cultured in Dulbecco's modified Eagle's medium (DMEM) with 10% fetal bovine serum (FBS) and HepG2, Hep3B, SNU387 were cultured in Eagle's Minimum Essential Medium (EMEM) supplemented with FBS at 37° C. in a 5% CO₂ incubator. 100U/ml penicillin and 100 ug/ml streptomycin were also added in the medium as a complete medium.

Xenograft animal model. huh-7 cells+Matrigel suspension was injected subcutaneously (s.c.) into the flank of one hind leg of 6-8 weeks old male athymic BALB/C mice. Tumor size was monitored continually until reaching around 100 mm³. After that, mice were divided into several groups and received treatment according to study design respectively. Tumor size and body weight were measured daily or once two days. The terminal of the study was when tumor size reaches 3000 mm³, or severe necrosis, ulceration, or bleeding happens, in which case mice would be sacrificed humanely. Tumor and tissue collection were processed for further study as needed. MC38 was resuspended in PBS with density of 10⁶/ml. 6-8 weeks age C57BL6 male mice were inoculated 10⁶ MC38 cells each. Treatment initiated once tumors grow ranges from 50-100 mm³. Treatment was processed every 3 days. Tumor size and weight was monitored daily. Hydrogel was provided once weight loss larger than 10%. Mice were sacrificed on the day after 5 doses. Blood samples were collected for further study.

Dynamic light scattering (DLS). Particle sizes were tested with Nicomp Nano Z3000 DLS/ZLS system (Entegris). Samples were diluted to 50-100 ug/ml in PBS buffer before testing the particle size by dynamic light scattering measurements. The intensity weighting size distribution was reported by the instrument.

Cryogenic transmission electron microscopy (Cryo-EM). Cryo-EM images were acquired by the Center for Electron Microscopy and Analysis (CEMAS) in the Ohio State University. The suspension of lipid nanoparticles was placed on a copper grid coated with a carbon film. Then remove the excess liquid. Lipid nanoparticles were frozen rapidly in liquid ethane. Then the grid with samples was then transferred to the specimen chamber of the transmission electron microscope (TEM). The specimen chamber was cooled with liquid helium.

Real-time quantitative reverse transcriptase polymerase chain reaction (qRT-PCR). Total RNA was extracted from tissues or cells in vitro by TRIzol reagent (Thermo Fisher Sci). The cDNA was reverse transcribed from mRNA to cDNA with High-Capacity cDNA Reverse Transcription Kit (Fisher Scientific). Then cDNA was amplified 40 cycles with needed primers. The relative cDNA levels were quantified by SYBR Green with Applied Biosystems QuantStudio 7 and using the comparative Cr method (ΔΔC_{T} method) for analyzing mRNA expression. β-actin was used as an internal control.

Western blot analysis. Cells were grown in 100 mm Petri dishes for 48 hours before being lysed with lysis buffer (0.5M EDTA and 1% Triton X-100) in phosphate-buffered saline (PBS) containing protease inhibitor and phosphatase inhibitor cocktail. Protein was measured and diluted to same concentration before adding Laemmli with 10% β-ME and boiling for 5 minutes. Proteins then were added in each well of 10% Mini-protein TGX gels and run in Tris/Glycine buffers at 100V for 80 minutes. Then transfer proteins to the membrane in transfer buffer at 180A for at least one hour. Air dry the membrane and block with non-fat dry milk. Wash the membrane with TBST 3 times. Incubate the membrane with 1:1000 primary antibodies in TBS buffer with 5% BSA for overnight at 4° C., then incubate the membrane with 1:5000 secondary antibody in TBS with 5% BSA. Wash the membrane 3 times with TBST. Make imaging and analyze western blots after dripping ECL reagents.

In vitro enzyme-linked immunosorbent assay. The level of cytokines was analyzed using commercial ELISA kits from ThermoFisher for the detection of human IL-6, TNF-α, and IFN-γ after treatment. ELISA was processed following the protocol provided by manufacturer Plate was read at 450 nm wavelength with microplate reader.

Flow cytometry analysis. Cells were seeded in monolayers and incubated for overnight before harvested by 0.25% trypsin, 0.02% EDTA. Cells were resuspended and counted before washing in PBS with 0.5% BSA and collected by centrifugation. For the cell cycle distribution assay, cells were washed by cold PBS and then fixed with 75% ethanol for at least 12 hours. Dying and dead cells were stained with propidium iodide and excluded from analysis. Duplicates and dead cells were also excluded by gating with FSC and SSC.

Anti-tumor study in vivo. In the treatment of siRNA against Akt1 on nude mice study, mice were inoculated with 3*10⁶ huh-7 cells suspension via s.c. Once tumor grew to about 100 mm³, mice were divided into groups with similar average tumor size, respectively. siAkt1 was given every 3 days. After treatment, mice were monitored for 2 days and sacrificed. In the treatment of siRNA combined with Lenvatinib on nude mice study, mice were inoculated with 2*10⁶ huh-7 cells suspension via s.c. Once tumor grew to about 100 mm³, mice were divided into groups with similar average tumor size, respectively. After 6 dosages, animals were observed and sacrificed once tumor size grew larger than 3000 mm³, or severe necrosis, bleeding, or ulceration happens.

Statistical analysis. All in vitro study were processed in triplicate. In all in vivo study, each group had at least five mice respectively. Student's t-test was used to determine the significance of differences between the groups, data analysis with values of P<0.05 was considered as having statistically significance.

### Results

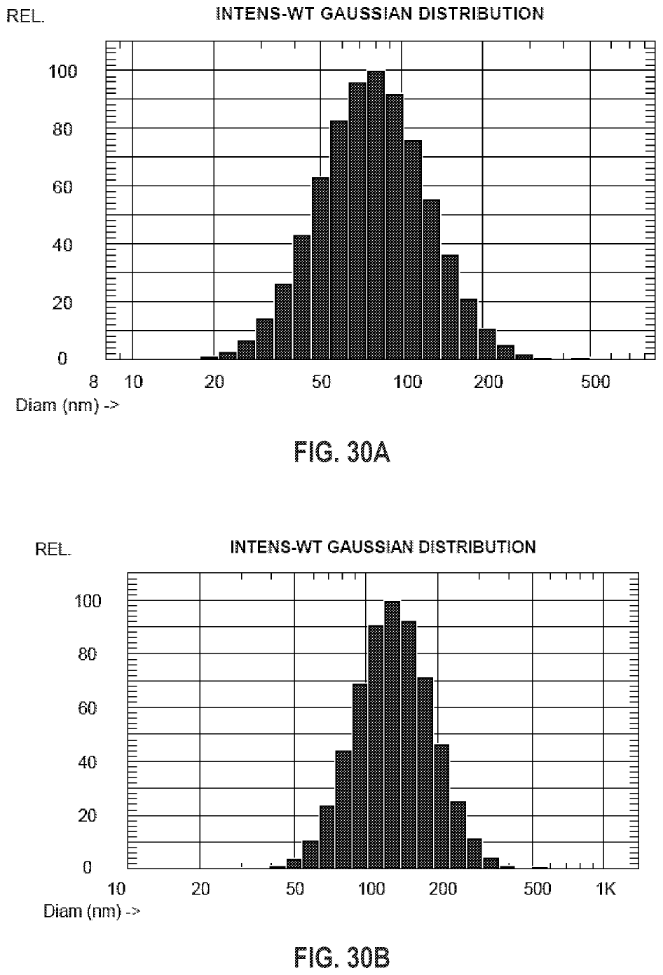

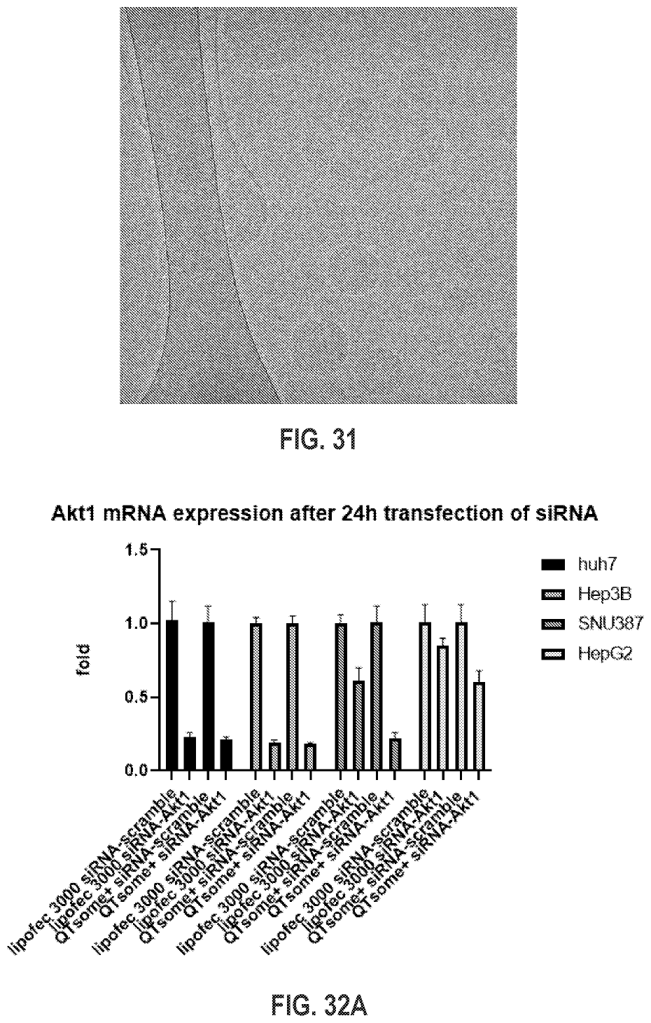

Physical characteristics of QTsome+ encapsulating siRNA. The particle size was measured by Dynamic light scattering (DLS). The siRNA was encapsulated by QTsome+ with drug lipid ratio 1:12. Ionizable lipids, cholesterol and PEG-DMG were mixed in ethanol and injected into acidic Citrate buffer quickly. After probe sonication and filtered by 45 nm membrane, the intensity weighting diameter of empty QTsome+ was 86.8 nm with a polydispersity index (PDI) of 0.189. Then siRNA aqueous solution was injected into empty QTsome+ solution under vortex. The intensity weighting diameter after titrated to pH 7.4 was 140.1 nm with a PDI of 0.234, which benefits to distribute to tumor sites through enhanced permeability and retention (EPR) effect. (FIGS. 30A and 30B). A single peak was observed in each sample and PDI value of two sample were relatively comparable, which indicates that QTsome+ was nearly monodisperse during and after the preparation. The intensity weighting diameter of encapsulated QTsome+ was larger than the empty one, showing that siRNA was encapsulated. The structure of siRNA-encapsulated QTsome+ was characterized via cryo-EM. According to the data provided by software, 99% of empty vesicle distribution are with less than 238.9 nm and 99% of siRNA-encapsulated vesicle distribution are with less than 322.1 nm.

As shown in FIG. 31, QTsome+ shows bilamellar structure with smooth particle curvature. Some small vesicles showing lamellar structure also presents in the lipid nanoparticle interior, which indicates that siRNA was surrounded by ionizable or cationic lipids due to the interaction between positive charge of lipid head group and negative charge of nucleic acid, forming a water/oil/water system.

TABLE 2
Cumulative Result of particle distribution:

| | Empty QTsome+ | Encapsulated QTsome+ |
|---|---|---|
| 25% of distribution< | 60.6 | 105.4 |
| 50% of distribution< | 86.8 | 140.1 |
| 75% of distribution< | 114.6 | 176.4 |
| 80% of distribution< | 123.1 | 187.2 |
| 90% of distribution< | 149.3 | 219.3 |
| 99% of distribution< | 238.9 | 322.1 |

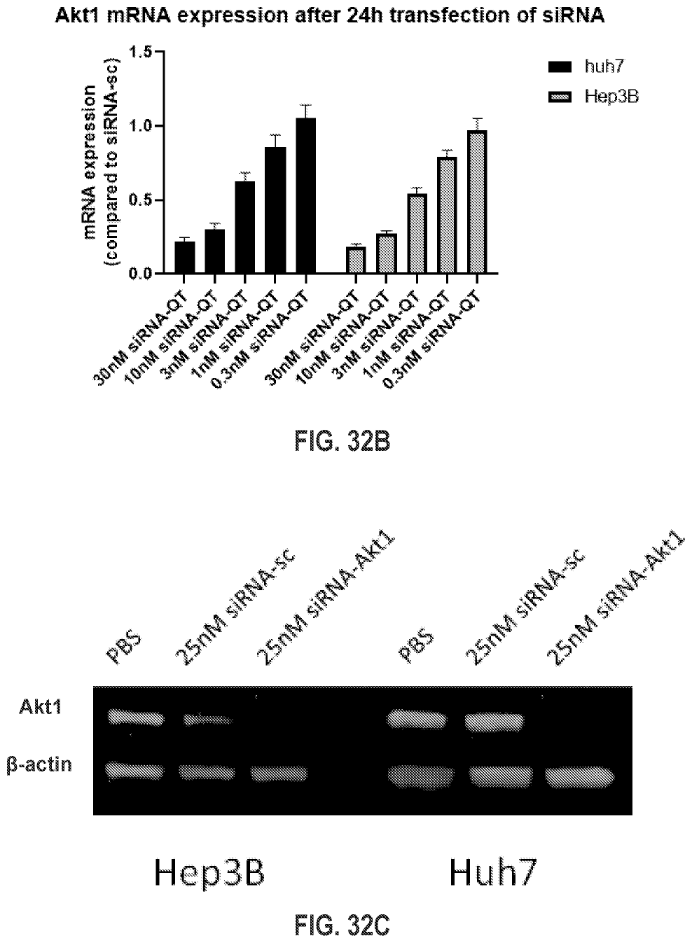

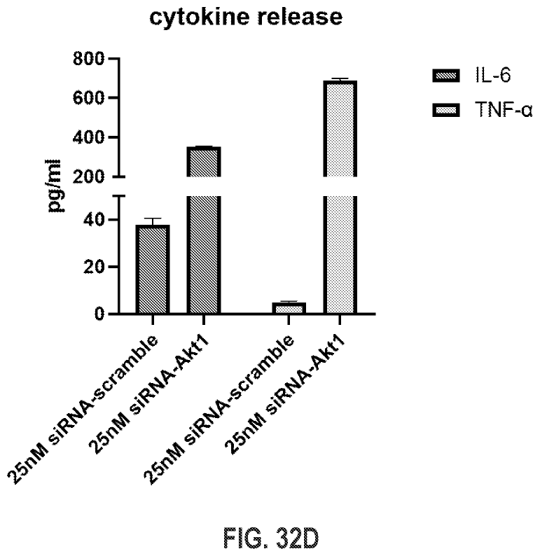

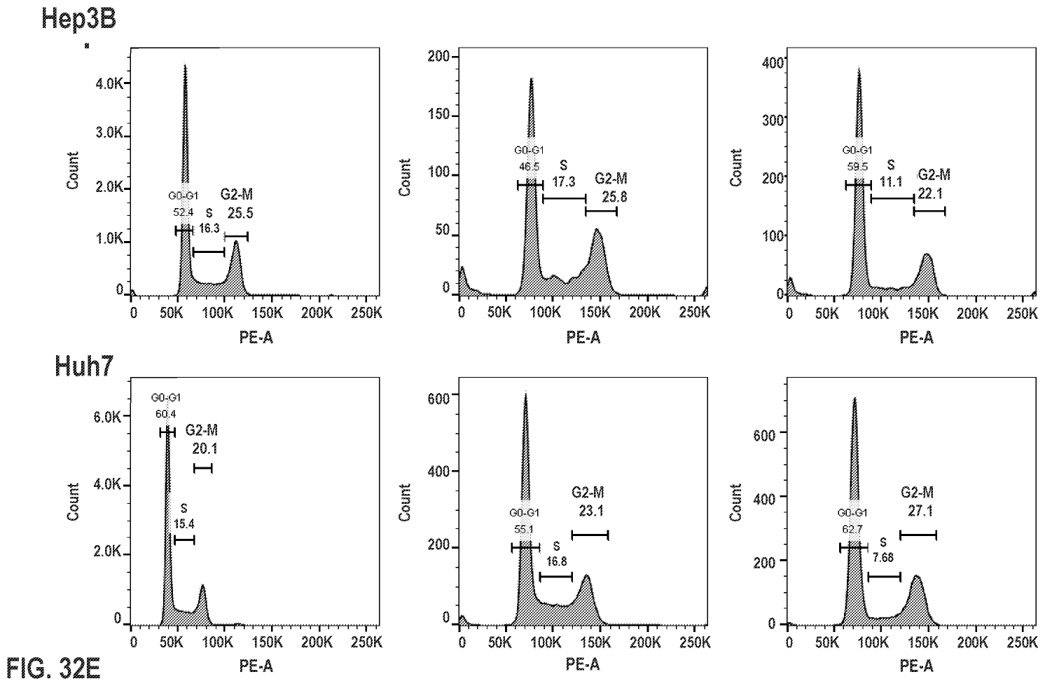

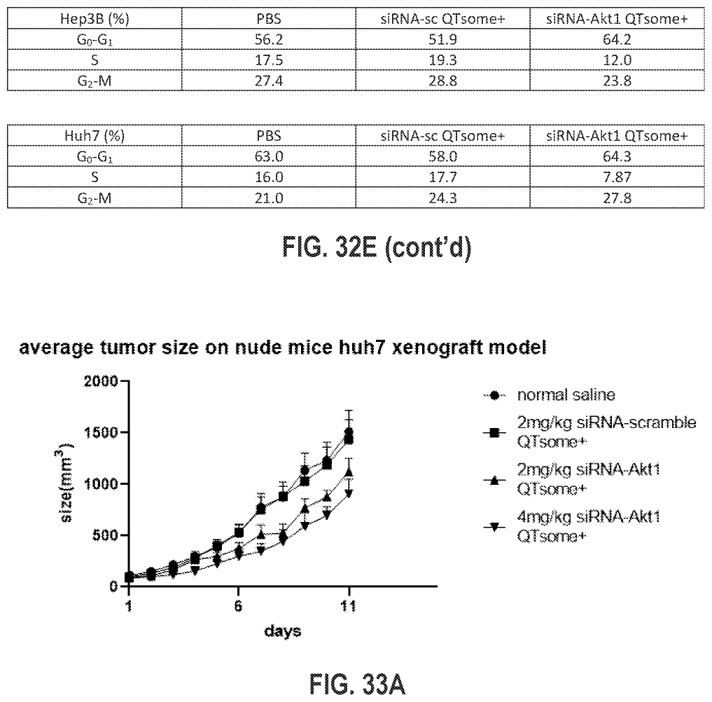

siRNA-Akt1 downregulate Akt1 gene and protein level in HCC cell line. The effect of siRNA to suppress Akt1 mRNA was initially evaluated on huh7, Hep3B, SNU387 and HepG2 cell cultures. siRNA was delivered via either lipofectamine 3000 reagent or QTsome+ and transfected into cells (FIG. 32A). After 24 hours transfection at 25 nM dose of siRNA-scramble or siRNA-Akt1, mRNA was extracted, and reverse transcribed to cDNA. mRNA expression was quantified by SYBR Green with Applied Biosystems QuantStudio 7 and using the comparative Cr method for analysis. For each vehicle, siRNA-scramble functions as an internal control of siRNA-Akt1 to diminish the effect of vehicle on gene expression. There is significant downregulation of Akt1 mRNA in huh7 and Hep3B cell line treated with siRNA-Akt1, no matter which vesicle was used to deliver siRNA. In terms of SNU387, the Akt1 downregulation in the cell treated with QTsome+ is also significant while the transfection efficiency of siRNA encapsulated by lipofectamine 3000 is not as good as by QTsome+. Although slight downregulation of Akt1 was observed, neither lipofectamine 3000 nor QTsome+ performed good transfection efficiency on HepG2 cells, indicating that HepG2 is not an optimum model for siRNA-Akt1 transfection. Different dose of siRNA was also tested on huh-7 and Hep3B to evaluate the relationship between Akt1 downregulation and dose (FIG. 32B). From 0.3 nM to 30 nM, higher dose leads to more downregulation level of mRNA, while there is almost no downregulation observed at 0.3 nM.

Western blot was then processed to evaluate the effect of siRNA-Akt1 on the Akt1 protein expression. Cells were transfected 48 hours with PBS, 25 nM siRNA-scramble and 25 nM siRNA-Akt1 encapsulated by QTsome+ respectively. According to FIG. 32C, the protein expression of Akt1 was suppressed on huh-7, Hep3B and SNU387 cell lines after treatment of siRNA-Akt1 QTsome+, compared to the groups treated with PBS or siRNA-scramble. It demonstrates the siRNA-Akt1 can suppress Akt1 protein expression in these cell lines and evaluates that QTsome+ can deliver siRNA transmembrane to the cytosol and target the mRNA efficiently.

It is known that proinflammatory cytokines interleukin (IL)-6 and tumor necrosis factor (TNF)-α play essential roles as mediators of immunity and inflammation, as well as promoting macrophage polarization and inflammatory activity of macrophage. See FIG. 32D.

Cell cycle was evaluated by flow cytometry. Cells were transfected with PBS, siRNA-scramble or siRNA-Akt1 at 25 nM for 48 hours before fixing and staining. Separation of cells in G0/G1, S and G2/M phase was determined by the linear fluorescence intensity after staining with propidium iodide (PI). As shown in FIG. 32E, siRNA-Akt1 induces cell-cycle arrest in S phase. The proportions of cells in G0/G1 phase decreased, accompanied with slight increase of the proportions of cells in the S phase. In huh-7, the cell proportions of G0/G1 phase decrease from 59.5% to 46.5% with the treatment of siRNA-Akt1, while the S phase increased from 11.1% to 17.3%, compared with PBS. Similar trends were also observed in Hep3B and SNU387 cells. It indicates that downregulation of Akt1 by siRNA is responsible for the S phase accumulation in huh-7, Hep3B and SNU387 cells.

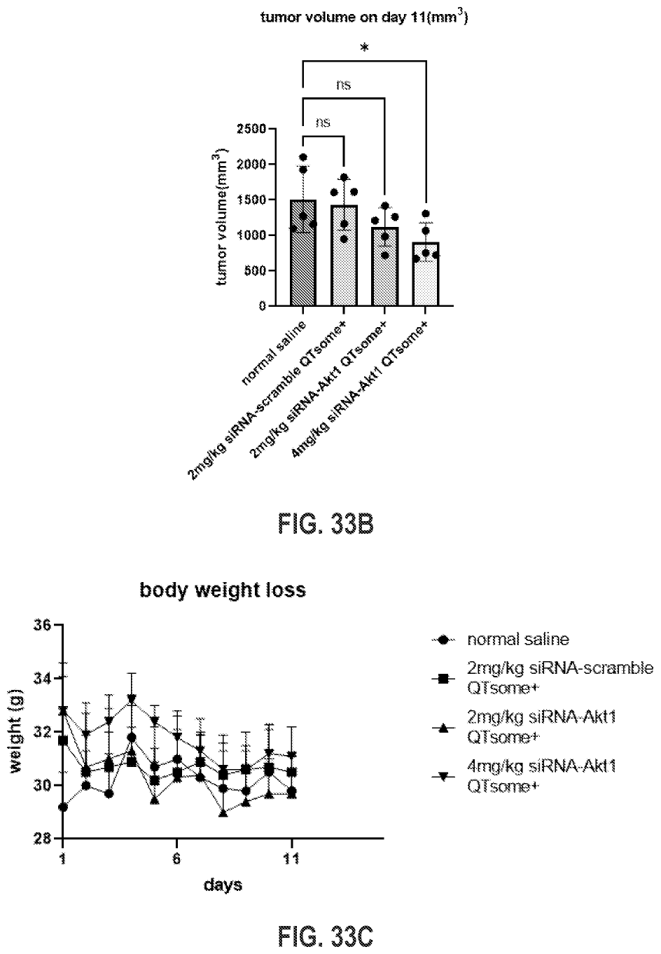

In vivo treatment of si-Akt1 results in anti-tumor activity on huh7 xenografts without severe side effects on nude mice. To examine the in vivo efficacy of siRNA-Akt1, nude mice bearing huh7 xenografts were treated with normal saline, siRNA-scramble QTsome+, or siRNA-Akt1 QTsome+ for 11 days. 8-10 weeks old nude male mice were inoculated with 3*10⁶ huh7 cells. Treatment initiated once average tumor size grew to 100-150 mm³. According to the animal data, siRNA-Akt1 encapsulated by QTsome+ can significantly suppress tumor growth at both 2 mg/kg and 4 mg/kg dose, of which the tumor growth inhibition (TGI) rate is 25.90% and 40.07% respectively. The anti-tumor effect of 4 mg/kg siRNA-Akt1 is better than 2 mg/kg, which means there is positive correlation between dose and effect to some extent (FIG. 33A). Although the mechanism of the anti-tumor activity is not clear, but it demonstrates that Akt1 is a good target. The hypothesis is siRNA targets to macrophages and induces the macrophage polarization, subsequently changing the tumor microenvironment. The average tumor size of mice treated with siRNA-scramble has no significant difference compared to those treated with normal saline, indicating that QTsome+ has minimum effect on tumor suppression. The average body weight of all the groups treated with siRNA QTsome+ decreases with relatively similar level, indicating that QTsome+ has slight toxicity as a vehicle, while Akt1 downregulation has negligible effect on body weight loss. There were no severe side effects such as dizziness, asthenia, dehydration, or ulceration happening (FIG. 33B).

TABLE 3
Tumor suppression analysis on Day 11 of male athymic
BALB/C mice inoculated with huh7 xenograft model.

| | Tumor Size (mm³) on | TGI (%) on | |
|---|---|---|---|
| Treatment Description | Day 11 | Day 11 | P-value |
| Normal Saline | 1512.0 | / | / |
| 2 mg/kg siRNA-sc | 1432.2 | 5.28% | 0.9692 |
| QTsome+ | | | |
| 2 mg/kg siRNA-Akt1 | 1120.4 | 25.90% | 0.2264 |
| QTsome+ | | | |
| 4 mg/kg siRNA-Akt1 | 906.1 | 40.07% | 0.0392 |
| QTsome+ | | | |

siRNA-Akt1 can suppress the growth of MC38 xenograft on C57BL6 mice. To evaluate the anti-tumor efficacy of siRNA-Akt1 on murine cell line MC38, pharmacodynamic study in vivo was processed on C57BL6 mice. Murine-derived cells were resuspended in PBS with density of 10⁶/ml. 6-8 weeks age C57BL6 male mice were inoculated 10⁶ MC38 cells each. Treatment initiated once tumors grow ranges from 50-100 mm³. Treatment was processed every 3 days. Tumor size and weight was monitored daily. Hydrogel was provided once weight loss larger than 10%. Mice were sacrificed 2 days after 4 doses. Blood samples were collected for further study.

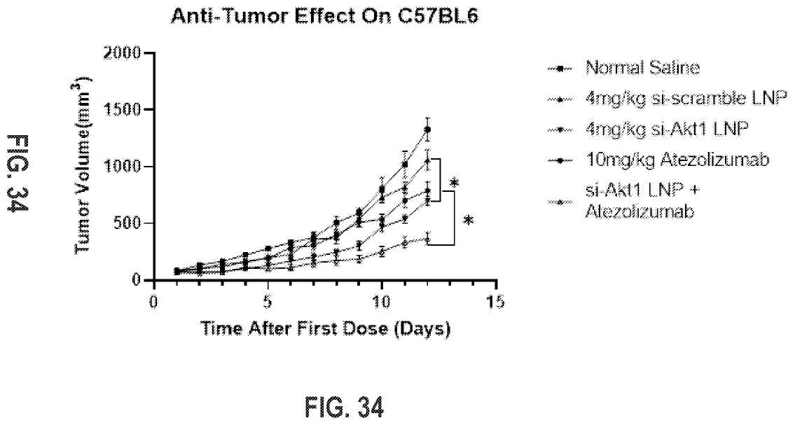

According to FIG. 34, the siRNA-Akt1 has significant difference compared to siRNA-scramble at 4 mg/kg dose, indicating that downregulation of Akt1 can induce anti-tumor effect in vivo. This may be caused by the induction of macrophage M1 subtype which suppress the growth of exogenous tumor cells. Compared to group treated with normal saline, siRNA-scramble shows slight tumor inhibition, but there is no statistically significant difference, implying the vehicle may have some influence on tumor suppression. Combination of siRNA-Akt1 and anti-PDL1 monoclonal antibody Atezolizumab shows satisfying anti-tumor effect (72.4% TGI compared to normal saline), and the significant difference between the combination group and group treated with Atezolizumab alone demonstrates downregulation of Akt1 can promote the tumor suppression efficacy of anti-PDL1, which illustrates that inhibition of both Akt1 and PDL I simultaneously can be a good thinking for cancer therapy.

These results are further described in the attached Appendix, which is hereby incorporated by reference herein in its entirety.

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A pharmaceutical composition comprising a lipid particle encapsulating an active agent, the lipid particle comprising:
one or more cationic lipids;
one or more ionizable lipids;
one or more neutral lipids; and
one or more PEGylated lipids;
wherein the one or more cationic lipids and the one or more ionizable lipids are present in the lipid particle in amount effective to produce a pKa apparent of from 6 to 10, such as from 6 to 8, as determined by a TNS pKa assay.

2. A pharmaceutical composition comprising a lipid particle encapsulating an active agent, the lipid particle comprising:
one or more cationic lipids;
one or more ionizable lipids;
one or more neutral lipids; and
one or more PEGylated lipids;
wherein the one or more cationic lipids and the one or more ionizable lipids are present in the lipid particle in amounts that satisfy the expression below
$$6 < {pKa}_{0} + k * Q / T < 10$$
were
pKa₀ represents the pKa of the ionizable lipid;
k represents the empirical constant determined by a TNS pKa assay
Q represents the mole % of the cationic lipid; and
T represents the mole % of the ionizable lipid.

3. The composition of claim 2, wherein the one or more cationic lipids and the one or more ionizable lipids are present in the lipid particle in amounts that satisfy the expression below
$$6 < {pKa}_{0} + k * Q / T < 8$$
were
pKa₀ represents the pKa of the ionizable lipid;
k represents the empirical constant determined by a TNS pKa assay
Q represents the mole % of the cationic lipid; and
T represents the mole % of the ionizable lipid.

4. The composition of any of claims 2-3, wherein Q/T is from greater than 0 to 1.

5. The composition of any of claims 1-4, wherein the one or more cationic lipids are present in the lipid particle in an amount of from greater than 0 mol % to 10 mol %, based on the total components forming the lipid particle.

6. The composition of claim 5, wherein the one or more cationic lipids are present in the lipid particle in an amount of from 0.5 mol % to 5 mol %, based on the total components forming the lipid particle.

7. The composition of claim 5, wherein the one or more cationic lipids are present in the lipid particle in an amount of from 4 mol % to 8 mol %, based on the total components forming the lipid particle.

8. The composition of any of claims 1-7, wherein the one or more ionizable lipids are present in the lipid particle in an amount of from 20 mol % to 65 mol %, based on the total components forming the lipid particle.

9. The composition of any of claims 1-8, wherein the one or more neutral lipids are present in the lipid particle in an amount of from 35 mol % to 80 mol %, based on the total components forming the lipid particle.

10. The composition of any of claims 1-9, wherein the one or more PEGylated lipids are present in the lipid particle in an amount of from greater than 0 mol % to 5 mol %, based on the total components forming the lipid particle.

11. A pharmaceutical composition comprising a lipid particle encapsulating an active agent, the lipid particle comprising:
from greater than 0 mol % to 10 mol % one or more cationic lipids;
from 20 mol % to 65 mol % one or more ionizable lipids;
from 35 mol % to 80 mol % one or more neutral lipids; and
from greater than 0 mol % to 5 mol % one or more PEGylated lipids.

12. The composition of claim 11, wherein the one or more cationic lipids and the one or more ionizable lipids are present in the lipid particle in amounts that satisfy the expression below
$$6 < {pKa}_{0} + k * Q / T < 10$$
were
pKa₀ represents the pKa of the ionizable lipid;
k represents an empirical constant determined by a TNS pKa assay
Q represents the mole % of the cationic lipid; and
T represents the mole % of the ionizable lipid.

13. The composition of claim 12, wherein the one or more cationic lipids and the one or more ionizable lipids are present in the lipid particle in amounts that satisfy the expression below
$$6 < {pKa}_{0} + k * Q / T < 8$$
were
pKa₀ represents the pKa of the ionizable lipid;
k represents the empirical constant determined by a TNS pKa assay
Q represents the mole % of the cationic lipid; and
T represents the mole % of the ionizable lipid.

14. The composition of any of claims 11-13, wherein the one or more cationic lipids are present in the lipid particle in an amount of from 0.5 mol % to 10 mol %, based on the total components forming the lipid particle.

15. The composition of any of claims 11-14, wherein the one or more cationic lipids are present in the lipid particle in an amount of from 0.5 mol % to 3.5 mol %, based on the total components forming the lipid particle.

16. The composition of any of claims 11-14, wherein the one or more cationic lipids are present in the lipid particle in an amount of from 4 mol % to 8 mol %, based on the total components forming the lipid particle.

17. The composition of any of claims 1-16, wherein the one or more ionizable lipids are present in the lipid particle in an amount of from 30 mol % to 50 mol %, based on the total components forming the lipid particle.

18. The composition of any of claims 1-17, wherein the one or more ionizable lipids comprise a lipid headgroup comprising a tertiary amine.

19. The composition of any of claims 1-18, wherein the one or more ionizable lipids comprise N,N-dimethyl-2,3-dioleyloxypropylamine (DODMA), [(4-hydroxybutyl)azanediyl]di(hexane-6,1-diyl)bis(2-hexyldecanoate) (ALC-0315), 9-heptadecanyl 8-{(2-hydroxyethyl)[6-oxo-6-(undecyloxy)hexyl]amino}octanoate (SM-102), DLin-MC3-DMA, DLin-KC2-DMA, 1-(2,3-bis(((9Z,12Z)-octadeca-9,12-dien-1-yl)oxy)propyl)pyrrolidine (A066), or any combination thereof.

20. The composition of any of claims 1-19, wherein the one or more neutral lipids are present in the lipid particle in an amount of from 30 mol % to 50 mol % of the total components forming the lipid particle.

21. The composition of any of claims 1-20, wherein the one or more neutral lipids comprise dipalmitoylphosphatidylcholine (DPPC), dioleoylphosphatidylethanolamine (DOPE), palmitoyloleoylphosphatidylcholine (POPC), egg phosphatidylcholine (EPC), distearoylphosphatidylcholine (DSPC), cholesterol, or any combination thereof.

22. The composition of any of claims 1-21, wherein the one or more PEGylated lipids are present in the lipid particle in an amount of from 0.5 mol % to 3 mol % of the total components forming the lipid particle.

23. The composition of any of claims 1-22, wherein the one or more PEGylated lipids comprise a PEG-ditetradecylacetamide, a PEG-myristoyl diglyceride, a PEG-diacylglycerol, a PEG dialkyloxypropyl, a PEG-phospholipid, a PEG-ceramide, or any combinations thereof.

24. The composition of any of claims 1-23, wherein the one or more cationic lipids comprise a lipid headgroup comprising a quaternary amine.

25. The composition of any of claims 1-24, wherein the one or more cationic lipids comprise DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA (2,3-dioleyloxy-N-[2-(spermine carboxamido)ethyl]-N,N-dimethyl-1-propanaminium trifluoroacetate), DORIE (N-[1-(2,3-dioleyloxypropyl)]-N,N-dimethyl-N-hydroxyethylammonium bromide), DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio) propane, DC-6-14: O,O-ditetradecanoyl-N-.alpha.-trimethylammonioacetyl) diethanolamine chloride, CLIP 1: rac-[(2,3-dioctadecyloxypropyl) (2-hydroxyethyl)]-dimethylammonium chloride, CLIP6: rac-[2 (2,3-dihexadecyloxypropyloxymethyloxy)ethyl]-trimethylammonium, CLIP9: rac-[2 (2,3-dihexadecyloxypropyloxysuccinyloxy)ethyl]-trimethylammonium, oligofectamine, lipids described in U.S. Pat. No. 5,049,386, N-[1-(2,3-dioleyloxypropyl)]-N,N-dimethyl-N-hydroxyethylammonium bromide (DORIE), 2,3-dioleyloxy-N-[2-(spermine carboxamido)ethyl]-N,N-dimethyl-1-propanaminium trifluoroacetate (DOSPA), and the like as disclosed in International Publication Nos. WO91/16024 and WO97/019675; DLinDMA and the like as disclosed in International Publication No. WO2005/121348; and DLin-K-DMA and the like as disclosed in International Publication No. WO2009/086558; and (3R,4R)-3,4-bis((Z)-Hexadec-9-enyloxy)-1-methylpyrrolidine, and N-Methyl-N, N-bis(2-((Z)-octadec-6-enyloxy)ethyl)amine and the like as disclosed in International Publication No. WO2011/13636, or any combination thereof.

26. The composition of any of claims 1-25, wherein the lipid particles have an average diameter of less than 1 micron, such as from 50 nm to 750 nm, 50 nm to 250 nm, from 50 nm to 200 nm, from 50 nm to 150 nm, or from 50 nm to 100 nm.

27. The composition of any of claims 1-26, wherein the lipid particles have a polydispersity index (PDI) of less than 0.4.

28. The composition of any of claims 1-27, wherein the active agent comprises a nucleic acid.

29. The composition of claim 28, wherein the nucleic acid comprises siRNA, mRNA, or any combination thereof.

30. A method of delivering an active agent to a cell, the method comprising contacting the cell with the composition of any of claims 1-29.

31. A method for in vivo delivery of an active agent to a cell, said method comprising administering to a mammalian subject the composition of any of claims 1-29.

32. The method of claim 31, wherein the mammal is a human.

33. The method of any of claims 31-32, wherein the administration is intravenous or intramuscular.

34. A method of systemically administering an active agent to a subject in need thereof, the method comprising intraveneously injecting a pharmaceutical composition comprising a lipid particle encapsulating the active agent, the lipid particle comprising:
from 0.5 mol % to 8 mol % one or more cationic lipids;
from 20 mol % to 65 mol % one or more ionizable lipids;
from 35 mol % to 80 mol % one or more neutral lipids; and
from greater than 0 mol % to 5 mol % one or more PEGylated lipids.

35. The method of claim 34, wherein the active agent comprises an anticancer agent.

36. A method of administering an active agent to a liver of a subject, the method comprising intraveneously injecting a pharmaceutical composition comprising a lipid particle encapsulating the active agent, the lipid particle comprising:
from 0.5 mol % to 3 mol % one or more cationic lipids;
from 20 mol % to 65 mol % one or more ionizable lipids;
from 35 mol % to 80 mol % one or more neutral lipids; and
from greater than 0 mol % to 5 mol % one or more PEGylated lipids.

37. The method of claim 36, wherein the active agent comprises an anticancer agent, such as an active agent for the treatment of liver cancer.

38. A method of administering an active agent to a solid tumor in a subject, the method comprising intraveneously injecting a pharmaceutical composition comprising a lipid particle encapsulating the active agent, the lipid particle comprising:
from 3 mol % to 6 mol % one or more cationic lipids;
from 20 mol % to 65 mol % one or more ionizable lipids;
from 35 mol % to 80 mol % one or more neutral lipids; and
from greater than 0 mol % to 5 mol % one or more PEGylated lipids.

39. The method of claim 36, wherein the active agent comprises an anticancer agent.

40. The method of any of claims 38-39, wherein the method targets the tumor neovasculature.

41. A method of administering an active agent to a lung of a subject, the method comprising intraveneously injecting a pharmaceutical composition comprising a lipid particle encapsulating the active agent, the lipid particle comprising:
from 6 mol % to 10 mol % one or more cationic lipids;
from 20 mol % to 65 mol % one or more ionizable lipids;
from 35 mol % to 80 mol % one or more neutral lipids; and
from greater than 0 mol % to 5 mol % one or more PEGylated lipids.

42. The method of claim 41, wherein the active agent comprises an anticancer agent, such as an active agent for the treatment of lung cancer.

43. A method of locally administering an active agent to a subject in need thereof, the method comprising intramuscularly injecting a pharmaceutical composition comprising a lipid particle encapsulating the active agent, the lipid particle comprising:
from greater than 0 mol % to 5 mol % one or more cationic lipids;
from 20 mol % to 65 mol % one or more ionizable lipids;
from 35 mol % to 80 mol % one or more neutral lipids; and
from greater than 0 mol % to 5 mol % one or more PEGylated lipids.

44. The method of claim 43, wherein the active agent comprises a nucleic acid.

45. The method of claim 44, wherein the nucleic acid comprises siRNA, mRNA, or any combination thereof.

46. The method of any of claims 43-45, wherein the active agent comprises a vaccine.
